(19) United States
(12) Patent Application Publication
(10) Pub. No.: US 2007/0087977 A1
(43) Pub. Date: Apr. 19, 2007

(54) METHODS AND COMPOSITIONS FOR TREATING PAIN

(76) Inventor: Wendye Robbins, San Francisco, CA (US)

Correspondence Address: WILSON SONSINI GOODRICH & ROSATI, 650 PAGE MILL ROAD, PALO ALTO, CA, 94304-1050, US

(21) Appl. No.: 11/553,924

(22) Filed: Oct. 27, 2006

Related U.S. Application Data
(63) Continuation-in-part of application No. 11/281,771, filed on Nov. 16, 2005.
(60) Provisional application No. 60/628,646, filed on Nov. 16, 2004.

(51) Int. Cl. A61K 31/7048; A61K 31/7008; A61K 31/573; A61K 31/551; A61K 31/485; A61K 31/55; A61K 31/445; A61K 31/353; A61K 31/195

(52) U.S. Cl. 514/23; 514/171; 514/220; 514/27; 514/456; 514/282; 514/561; 514/217; 514/317

(57) ABSTRACT

Methods and compositions are described for the modulation of central nervous system and/or fetal effects of substances. Methods and compositions are described for the modulation of efflux transporter activity to increase the efflux of drugs and other compounds out of a physiological compartment and into an external environment. In particular, the methods and compositions disclosed herein provide for the increase of efflux transporter activity at blood-brain, blood-CSF and placental-maternal barriers to increase the efflux of drugs and other compounds from physiological compartments, including central nervous system and fetal compartments.

### CROSS-REFERENCE

This application is a continuation-in-part of U.S. application Ser. No. 11/281,771 filed on Nov. 16, 2005, which claims the benefit of U.S. Provisional Application No. 60/628,646, filed Nov. 16, 2004, both of which applications are incorporated herein by reference in its entirety.

### BACKGROUND OF THE INVENTION

Although anatomical blood barrier structures, such as the blood-brain barrier (BBB) and placenta, function as a block, for example, to isolate the central nervous system from the systemic blood circulation, pharmaceutical agents, such as anesthetic agents, often cross the barrier causing systemic side-effects rather than a desired localized action. In addition, BBB and placental barrier can be compromised by disease states and therapeutic treatments, causing unwanted agents to cross across the barrier and adversely affect brain structures or a developing fetus. Therefore, there is a need in the field to find methods and modulators that block entry of unwanted agents into the central nervous system and/or the placenta.

### SUMMARY OF THE INVENTION

In one aspect, the invention provides compositions including BBB transport protein activator. In some embodiments of this aspect, the invention provides a pharmaceutical composition including an analgesic agent and a blood brain barrier (BBB) transport protein activator and a pharmaceutically acceptable excipient, where the analgesic agent is present in an amount sufficient to produce an analgesic effect, and wherein the BBB transport protein activator is present in an amount sufficient to reduce a central nervous system (CNS) effect of the analgesic agent.

In some embodiments of the compositions of the invention, the BBB transport protein includes an ABC transport protein. In some embodiments, the CNS effect includes drowsiness, impaired concentration, sexual dysfunction, sleep disturbances, habituation, dependence, alteration of mood, respiratory depression, nausea, vomiting, dizziness memory impairment, neuronal dysfunction, neuronal death, visual disturbance, impaired mentation, tolerance, addiction, hallucinations, lethargy, myoclonic jerking, endocrinopathies, and combinations thereof. In some embodiments of the invention, a therapeutic effect of the therapeutic agent is increased at least about 5% compared to the therapeutic effect without the BBB transport protein activator, when the composition is administered to an animal. In some embodiments, the ABC transport protein includes a P-gP.

In some embodiments of the compositions of the invention, the analgesic includes oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol. In some embodiments, the analgesic includes oxycodone or gabapentin. In some embodiments, the analgesic is oxycodone. In some embodiments, the analgesic is gabapentin. In some embodiments of the invention, the BBB transport protein activator includes a polyphenol. In some embodiments, the BBB transport protein activator includes a flavonoid. In some embodiments, the BBB transport protein activator includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the BBB transport protein activator is quercetin.

In some embodiments of the compositions of the invention, the analgesic includes oxycodone, gabapentin, pregabalin, hydrocodone, fentanyl, hydromorphine, levorphenol, morphine, methadone, tramadol and topiramate. In some embodiments, the analgesic includes oxycodone or gabapentin. In some embodiments, the analgesic is oxycodone. In some embodiments, the analgesic is gabapentin. In some embodiments, when the BBB transport protein activator is quercetin, the analgesic includes oxycodone or gabapentin. In some embodiments, the analgesic is oxycodone. In some embodiments of the invention, the oxycodone and the quercetin are present in a molar ratio of about 0.002:1 to 0.1:1. In some embodiments, the oxycodone is present at about 5-160 mg and the quercetin is present at about 10-500 mg. In some embodiments, the oxycodone is present at about 80 mg and the quercetin is present at about 500 mg. In some embodiments of the invention, the composition further includes a pharmaceutically acceptable excipient.

In some embodiments of the compositions of the invention, the analgesic is gabapentin. In some embodiments, the gabapentin and the quercetin are present in a molar ratio of about 0.2:1 to 6:1. In some embodiments, the gabapentin is present at about 100-800 mg and the quercetin is present at about 50-5000 mg. In some embodiments, the gabapentin is present at about 300 mg and the quercetin is present at about 150 mg. In some embodiments of the invention, the composition further includes a pharmaceutically acceptable excipient. In some embodiments of the compositions of the invention, the analgesic and the BBB transport protein activator are present in a molar ratio of about 0.001:1 to about 10:1. In some embodiments, the analgesic and the BBB transport protein activator are present in a molar ratio of about 0.001:1 to about 10:1. In some embodiments, the analgesic is present at about 0.001 to 500 mg and the BBB transport protein is present at about 10 to 1000 mg. In some embodiments of the invention, the composition further includes a pharmaceutically acceptable excipient.

In some embodiments of the compositions of the invention, the central nervous system effect includes drowsiness, impaired concentration, sexual dysfunction, sleep disturbances, habituation, dependence, alteration of mood, respiratory depression, nausea, vomiting, dizziness memory impairment, neuronal dysfunction, neuronal death, visual disturbance, impaired mentation, tolerance, addiction, hallucinations, lethargy, myoclonic jerking, endocrinopathies, or combinations thereof. In some embodiments of the composition, the analgesic and the BBB transport protein activator are admixed.

In another aspect, the invention provides methods utilizing BBB transport protein activator. In some embodiments of this aspect, the invention provides a method of treating an animal for pain by administering to an animal in pain an effective amount of an analgesic agent and an amount of a BBB transport protein activator sufficient to reduce a central nervous system effect of the analgesic agent. In some embodiments of the methods of the invention, the BBB transport protein activator is administered in an amount sufficient to substantially eliminate a central nervous system effect of the analgesic compound. In some embodiments, the analgesic agent and the BBB transport protein activator are co-administered. In some embodiments, the analgesic compound and the BBB transport protein activator are administered admixed in a single composition. In some embodiments, the analgesic is present in the composition in an amount sufficient to produce an analgesic effect, and the BBB transport protein activator is present in the composition in an amount sufficient to reduce a central nervous system effect of the analgesic.

In some embodiments of the methods of the invention, the therapeutic agent is present in an amount sufficient to exert a therapeutic effect and the BBB transport protein modulator is present in an amount sufficient to decrease a CNS effect of the therapeutic agent by an average of at least about 10%, compared to the side effect without the BBB transport protein modulator. In some embodiments, the amount of analgesic agent is administered in an amount sufficient to produce an analgesic effect, and the amount is different than the amount sufficient to produce an analgesic effect in the absence of administration of the BBB transport protein modulator. In some embodiments, the amount of analgesic agent administered is lower than the amount sufficient to produce an analgesic effect in the absence of administration of the BBB transport protein modulator. In some embodiments, the administration is oral administration. In some embodiments, the administration is transdermal administration. In some embodiments, the animal in pain suffers from chronic pain. In some embodiments, the animal is a mammal. In some embodiments, the animal is a human.

In some embodiments of the methods of the invention, the BBB transport protein modulator includes an activator of P-gP. In some embodiments of the invention, the BBB transport protein activator includes a polyphenol. In some embodiments, the polyphenol is a flavonoid. In some embodiments, the flavonoid includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. In some embodiments, the analgesic includes oxycodone, gabapentin, pregabalin, hydrocodone, fentanyl, hydromorphine, levorphenol, morphine, methadone, tramadol or topiramate. In some embodiments, the analgesic includes oxycodone or gabapentin. In some embodiments, the analgesic is oxycodone. In some embodiments, the analgesic is gabapentin.

In some embodiments of the invention, where the flavonoid is quercetin, the analgesic includes oxycodone, gabapentin, pregabalin, hydrocodone, fentanyl, hydromorphine, levorphenol, morphine, methadone, tramadol or topiramate. In some embodiments, the analgesic includes oxycodone or gabapentin. In some embodiments, the analgesic is oxycodone. In some embodiments, the analgesic is gabapentin. In some embodiments, the analgesic compound and the BBB transport protein activator are administered together about once per day to about 6 times per day. In some embodiments, the administration continues for less than about 7 days. In some embodiments, the administration continues for more than about 6 days. In some embodiments, the methods of the invention further include administering to the animal in pain another therapeutic agent. In some embodiments, the other therapeutic agent includes antinausea agents, amphetamines, antianxiolytics, or hypnotics. In some embodiments of the invention, the molar ratio of the amount of analgesic agent administered and the amount of BBB transport protein modulator administered is about 0.001:1 to about 10:1.

In yet another aspect, the invention provides methods including co-administering BBB transport protein modulator and an analgesic agent. In some embodiments of this aspect, the invention provides a method of controlling chronic pain in an animal by co-administering to an animal suffering from chronic pain an effective amount of an analgesic agent; and an amount of a BBB transport protein modulator sufficient to prevent or delay the development of tolerance to the analgesic agent in the animal. In some embodiments of the methods of the invention, the animal is a mammal. In some embodiments, the mammal is a human. In some embodiments, the amount of the BBB transport protein modulator is sufficient to reduce the amount of analgesic necessary for pain relief. In some embodiments, the analgesic agent includes oxycodone, gabapentin, pregabalin, hydrocodone, fentanyl, hydromorphine, levorphenol, morphine, methadone, tramadol or topiramate. In some embodiments, the analgesic agent is oxycodone. In some embodiments, the analgesic agent is gabapentin. In some embodiments of the invention, the BBB transport protein modulator includes a polyphenol. In some embodiments, the polyphenol includes a flavonoid. In some embodiments, the flavonoid includes quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. In some embodiments, the analgesic agent and the BBB transport protein modulator are co-administered as admixed components of a single composition.

Another aspect of the invention is a method of identifying a transport modulator. A drug is administered in an appropriate animal model in the presence and absence of a test compound and the concentration of the drug in a biological sample is measured. The test compound is identified as a transport modulator if the concentration of the drug in the biological sample is lower in the presence of the test compound. In some embodiments, the biological sample may be intraventricular samples, amniotic fluid, chorionic samples or brain parenchymal samples. Moreover, the animal model may be a rodent, such as mice or rats, or a primate, horse, dog, sheep, goat, rabbit, or chicken. In other embodiments, the animal model possesses a mutant form of a blood brain and/or placental transporter.

Another aspect of the invention is a method for excluding a drug or compound from a physiological compartment by selectively increasing efflux of a drug or compound from the physiological compartment to an external environment, comprising co-administering to a patient an effective amount of a physiological compartment entry modulator with an effective amount of a drug or compound. In one embodiment, the physiological compartment is a central nervous system. In another embodiment, the physiological compartment is a fetal compartment.

Other objects, features and advantages of the methods and compositions described herein will become apparent from the following detailed description. It should be understood, however, that the detailed description and the specific examples, while indicating specific embodiments, are given by way of illustration only, since various changes and modifications within the spirit and scope of the invention will become apparent to those skilled in the art from this detailed description.

All publications, patents, and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustration of a blood-brain barrier and blood-CSF barrier.

FIG. 2 is an illustration of a portion of the molecular transporters in the blood brain barrier.

FIG. 3 is an illustration of placental circulation.

FIG. 4 is an illustration of one embodiment of the methods and compositions disclosed herein.

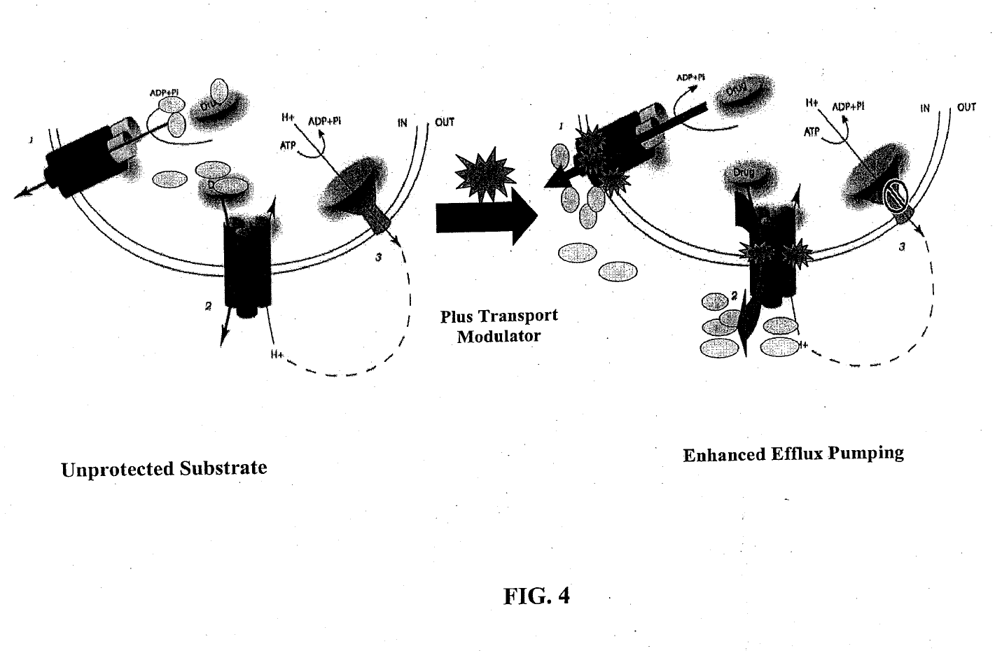

FIG. 5 is a graph that depicts an improvement in sleep in the patients.

FIG. 6 is a graph that depicts an improvement in concentration in the patients.

FIG. 7 is a graph that depicts an improvement in the worst pain in the last 24 hrs in the patients.

FIG. 8 is a graph that depicts an improvement in the pain at the time the patients were called.

FIG. 9 is a graph that depicts an improvement in the worst pain in the last 24 hrs for the opioid users.

FIG. 10 is a graph that depicts an improvement in the pain at the time of the call for the opioid users.

FIG. 11 is a graph that depicts a % change in the worst pain in the last 24 hrs in the opioid users.

FIG. 12 is a graph that depicts a % change in the pain at the time of the call in the opioid users.

FIG. 13 is a graph that depicts the worst pain in the last 24 hrs in the patients who were not on baseline meds and who were given quercetin only, quercetin with Vicodin, and Vicodin only.

FIG. 14 is a graph that depicts the pain at the time of the call in the patients who were not on baseline meds and who were given quercetin only, quercetin with Vicodin, and Vicodin only.

FIG. 15 is a graph that depicts global assessment of all the patients who were on opiate or MSD (membrane stabilizing drug) and modulator (Q) showing overall improvement in the pain.

FIG. 16 is a graph that depicts changes in means values for worst pain, pain now, sleep, and concentration for all patients taking analgesic and quercetin.

FIG. 17 is an illustration of active influx and efflux mechanisms across the blood-brain barrier.

### DETAILED DESCRIPTION OF THE INVENTION

Reference will now be made in detail to particularly preferred embodiments of the invention. Examples of the preferred embodiments are illustrated in the following Examples section.

Unless defined otherwise, all technical and scientific terms used herein have the same meaning as is commonly understood by one of skill in the art to which this invention belongs. All patents and publications referred to herein are incorporated by reference.

I. Introduction

The invention provides compositions and methods utilizing an agent that reduces or eliminates a central nervous system (CNS) and/or fetal effect of one or more substances. In some embodiments, the invention provides compositions and methods utilizing a combination of a therapeutic agent and an agent that reduces or eliminates a central nervous system (CNS) and/or fetal effect of the therapeutic agent. Typically, the CNS effect-decreasing agent is a modulator of a blood brain barrier (BBB) or a placental barrier transport protein. The terms “BBB transport protein modulator” and “BBB and/or placental transport protein modulator” are used interchangeably herein. The methods and compositions are useful in the treatment of an animal in need of treatment, where it is desired that one or more effects of the substance, e.g., therapeutic agent, in the central nervous system (CNS) or the developing fetus be reduced or eliminated. In embodiments further utilizing a therapeutic agent, the methods and compositions are useful in the treatment of an animal in need of treatment, where it is desired that one or more effects of the therapeutic agent, in the central nervous system (CNS) or the developing fetus be reduced or eliminated while one or more of the therapeutic effects (e.g., peripheral effects) of the agent are retained or enhanced.

In some embodiments of the invention, the therapeutic agent is an analgesic agent, such as an opiate or a non-opiate analgesic. In some embodiments of the invention, the therapeutic agent is a non-analgesic agent. The agent causing a decrease in the CNS effects of the therapeutic agent, e.g., a modulator of a BBB or placental barrier transport protein may be an activator or an inhibitor of the protein. The modulatory effect may be dose-dependent, e.g., some modulators act as activators in one dosage range and inhibitors in another. In some embodiments, a modulator of a BBB or placental barrier transport protein is used in a dosage wherein it acts primarily as an activator.

Typically, the use of the BBB or placental barrier transport protein modulator, e.g., activator, results in a decrease in one or more CNS and/or fetal effects of the therapeutic agent. The therapeutic effect(s) of the agent may be decreased, remain the same, or increase; however, in preferred embodiments, if the therapeutic effect is decreased, it is not decreased to the same degree as the CNS or fetal effects. It will be appreciated that a given therapeutic agent may have more than one therapeutic effect and or one or more CNS or fetal effects, and it is possible that the therapeutic ratio (in this case, the ratio of change in desired effect to change in undesired effect) may vary depending on which effect is measured. However, at least one therapeutic effect of the therapeutic agent is decreased to a lesser degree than at least one CNS effect of the therapeutic agent.

In addition, in some embodiments, one or more therapeutic effects of the agent is enhanced by use in combination with a BBB and/or placental transport protein modulator, while one or more CNS effects of the therapeutic agent is reduced or substantially eliminated. For example, in some embodiments, the analgesic effect of an analgesic agent is enhanced while one or more CNS effects of the agent is reduced or substantially eliminated.

Without being bound by theory, and as an example only of a possible mechanism, it is thought that the methods and compositions of the invention operate by reducing or eliminating the concentration of the therapeutic agent from the CNS (e.g., brain) and/or fetal compartment, while retaining or even increasing the effective concentration of the agent in the periphery. Agents that act at least in part by peripheral mechanisms may thus retain some or all of their activity, or even display enhanced therapeutic activity, while at the same time CNS and/or fetal effects are reduced or eliminated.

It will be appreciated that the therapeutic and/or CNS effects of an therapeutic agent may be mediated in part or in whole by one or metabolites of the therapeutic agent, and that a BBB or placental transport protein modulator that reduces or eliminates the CNS or fetal concentration of the therapeutic agent and/or of one or active metabolites of the therapeutic agent that produce CNS effects, while retaining or enhancing a peripheral concentration of the therapeutic agent and/or one or more metabolites producing a therapeutic effect, is also encompassed by the methods and compositions of the invention. In addition, a BBB or placental transport modulator itself may be metabolized to metabolites that have differing activities in the modulation of one or more BBB transport modulators, and these metabolites are also encompassed by the compositions and methods of the invention.

Hence, in some embodiments the invention provides compositions that include a therapeutic agent and a blood-brain barrier (BBB) and/or placental transport protein modulator, where the therapeutic agent is present in an amount sufficient to exert a therapeutic effect and the BBB and/or placental transport protein modulator is present in an amount sufficient to decrease a central nervous system (CNS) effect of the therapeutic agent when compared to the CNS effect without the BBB and/or placental transport protein modulator, when the composition is administered to an animal. The decrease in the CNS effect can be measurable. The BBB and/or placental transport protein modulator is a BBB and/or placental transport protein activator in some embodiments. In some embodiments the BBB and/or placental transport protein modulator is a modulator of ATP binding cassette (ABC) transport proteins. In some embodiments the BBB and/or placental transport protein modulator is a modulator of P-glycoprotein (P-gP).

In some embodiments, compositions of the invention include one or more than one therapeutic agent as well as one or more than one BBB transport protein modulators. One or more of the therapeutic agents may have one or more CNS effects which are desired to be decreased.

Compositions of the invention may be prepared in any suitable form for administration to an animal. In some embodiments, the invention provides pharmaceutical compositions.

In some embodiments, the invention provides compositions suitable for oral administration. In some embodiments, compositions are suitable for transdermal administration. In some embodiments, compositions are suitable for injection by any standard route of injection, e.g., intravenous, subcutaneous, intramuscular, or intraperitoneal. Compositions suitable for other routes of administration are also encompassed by the invention, as described herein.

BBB and/or placental transport protein modulators of use in the invention include any suitable BBB and/or placental transport modulators. In some embodiments, the BBB and/or placental transport protein modulator is one or more polyphenols. In some embodiments, the BBB and/or placental transport protein modulator is one or more flavonoids. In some embodiments, the BBB and/or placental transport protein modulator is quercetin.

Therapeutic agents of use in the invention include any suitable agent that produces a CNS and/or fetal effect that it is desired to reduce or eliminate, while retaining or enhancing a therapeutic effect of the agent. In some embodiments, the therapeutic agent is an analgesic agent. In some instances an effect, e.g., a CNS effect may be desirable in some cases and undesirable in others. For example, some analgesics also produce a sedating effect. In some instances, such a sedating effect may be desirable. For example, in the use of analgesics in terminal patients where the main object is to improve quality of the remaining period of life, a certain amount of sedation in addition to analgesia may be desirable. However, it is often desirable to decrease pain without altering mood or concentration, or with minimal alteration of mood or concentration. For example, in patients with chronic intractable pain who are otherwise in good health, it is often desired to achieve maximum alleviation of pain while having minimum sedation or effects on concentration. In the latter case, it is useful to decrease or eliminate the CNS effect of sedation while retaining the analgesic effect of the agent. It is within the invention to titrate the combination of dosage of therapeutic agent and of BBB and/or placental transport protein modulator in such a way as to obtain a ratio of therapeutic effect to CNS effect that is considered optimal. Thus, in some embodiments, one or more CNS effect of the therapeutic agent is reduced but not eliminated. In other embodiments, one or more CNS effects of the therapeutic agent is substantially eliminated. In some embodiments, the analgesic agent is an opiate. In some embodiments, the analgesic agent is a non-opiate.

In some embodiments the invention provides methods of treatment. In certain embodiments, the invention provides a method of treating a condition by administering to an animal suffering from the condition an effective amount of a therapeutic agent and an amount of an BBB transport protein modulator, e.g., activator, sufficient to reduce or eliminate a CNS effect of the therapeutic agent. In some embodiments the BBB transport protein modulator is a BBB transport protein activator. In some embodiments, the therapeutic agent is an analgesic agent, e.g., an opiate or a non-opiate analgesic. In certain embodiments the invention provides methods of treatment of pain, e.g., chronic pain, by administration of an analgesic, e.g., an opiate, without the development of tolerance and/or dependence to the analgesic, by co-administering a modulator of a BBB transport protein in combination with the analgesic, thereby preventing or delaying development of tolerance and/or dependence to the analgesic.

In some embodiments the invention provides methods of decreasing a CNS effect of an agent in an animal, e.g. a human, that has received an amount of the agent sufficient to produce a CNS effect by administering to the animal, e.g., human, an amount of a BBB transport protein modulator sufficient to reduce or eliminate the CNS effect. In certain embodiments, the agent is an anesthetic, e.g., a general anesthetic. In certain embodiments, the agent is a therapeutic agent or drug of abuse that has be administered in excess, e.g., in an overdose.

II. Blood-Brain Barrier and Placental Barrier

A. Blood Brain Barrier

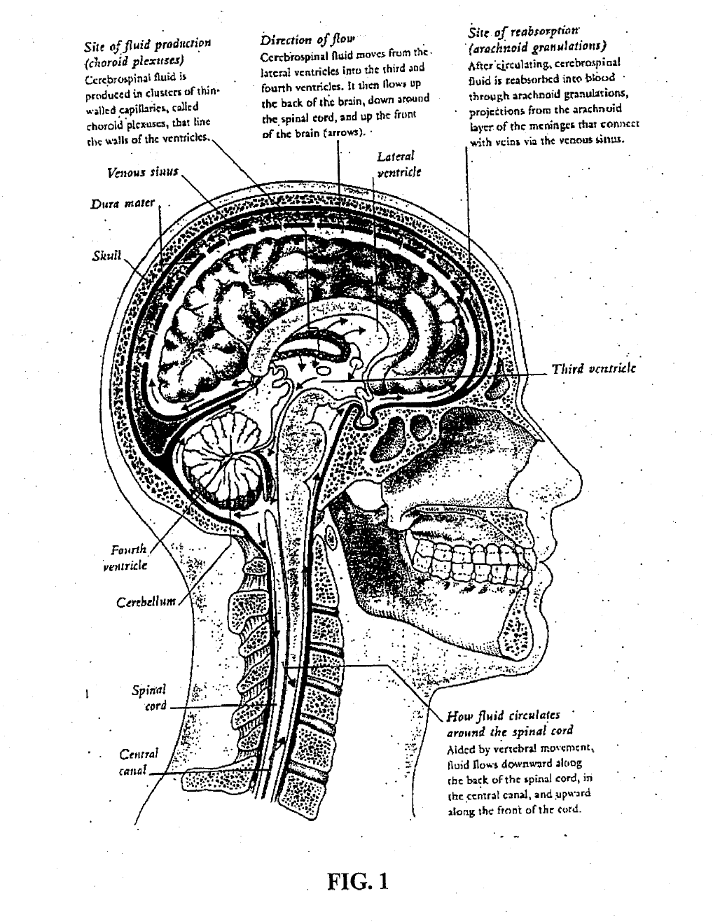

The access to the brain is controlled by at least two barriers, i.e., blood brain barrier (BBB) and blood-cerebrospinal fluid (CSF) barrier (see FIG. 1). As used herein, the term “blood brain-barrier” can encompass the blood-brain and blood-CSF barriers, unless otherwise indicated. The methods and compositions described herein are suitable for modulating the access of drugs into the brain. In some embodiments, the methods and compositions involve the modification of the blood brain barrier and/or blood-CSF barrier to prevent the entry of drugs into the central nervous system (CNS), e.g., by promoting efflux of the drugs from the CNS. In some embodiments, the compositions and methods of the invention utilize a modulator of a blood brain-barrier transport protein. In some embodiments, the compositions and methods of the invention utilize an activator of a blood brain-barrier transport protein.

The blood brain barrier is formed by tight intercellular junctions of brain capillary endothelial cells. The junctions are sealed by zonulae occludentes and tight junctions. The capillaries are covered by a continuous basal membrane enclosing pericytes, an intermittent cell layer, and the outer basal membrane is contacted by astrocytes. The electrical resistance across the endothelium is high, about 1500 to about 2000 Ω/cm².

The blood brain barrier regulates the transfer of substances between circulating blood and brain by facilitated transport and/or facilitated efflux. The interface on both luminal and abluminal surfaces contain physical and metabolic transporter components.

The exchange of substances between circulating blood and brain can be determined by evaluating octanol/H₂0 partition coefficient, facilitated transport, and/or facilitated efflux. The methods of measuring blood brain barrier integrity can be used to identify suitable central nervous system modulators for use in the methods and compositions described herein.

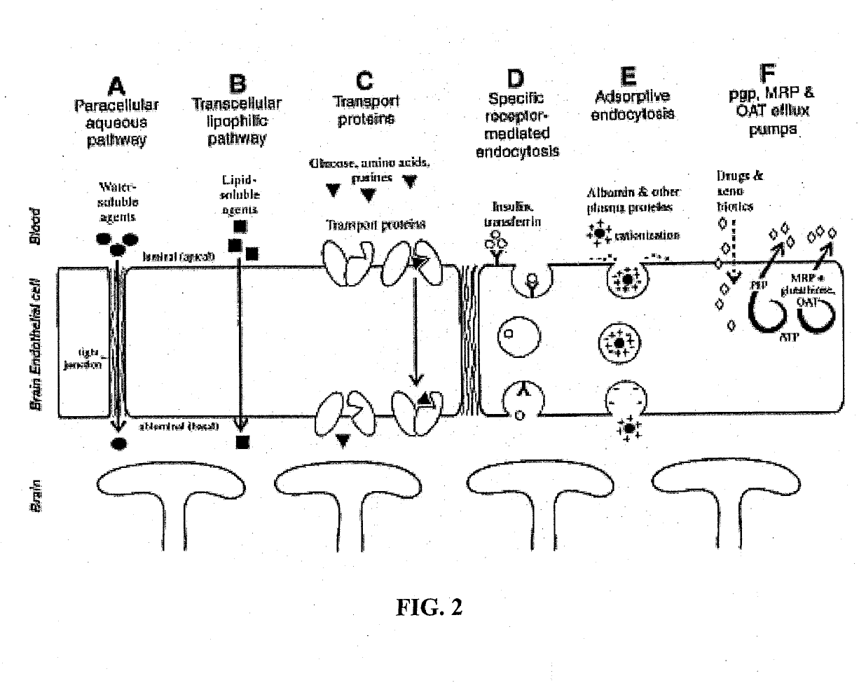

Various transporters exist to regulate rate of brain permeation for compounds with varying lipophilicity (see FIG. 2). Generally, hydrophilic nutrients, such as glucose and amino acids, are allowed entry into the physiological compartments of the methods and compositions disclosed herein. Conversely, compounds with low lipophilicity are pumped away from the physiological compartments by, for example, xenobiotic efflux transporters. These transporters are preferably modulated by the methods and compositions described herein to prevent entry of compounds and drugs into the central nervous system.

The blood CSF barrier is formed by the tight junctions of the epithelium of the choroid plexus and arachnoid membrane surrounding the brain and spinal cord. It is involved in micronutrient extraction, clearance of metabolic waste, and transport of drugs.

Mechanisms and routes of compounds into and out of brain include—paracellular aqueous pathway for water soluble agents, transcellular lipophilic pathway for lipid soluble agents, transport proteins for glucose, amino acids, purines, etc., specific receptor mediated endocytosis for insulin, transferrin, etc., adsorptive endocytosis for albumin, other plasma proteins, etc., and transporters (e.g., blood-brain barrier transport proteins) such as P-glycoprotein (P-gP), multi-drug resistance proteins (MRP), organic anion transporter (OAT) efflux pumps, gamma-aminobutyric acid (GABA) transporters and other transporters that modulate transport of drugs and other xenobiotics. Methods and compositions of the invention may involve modulation of one or more of these transporters. Preferably, the central nervous system modulators affect one or more of these mechanisms and routes to extrude drugs from the central nervous system.

The methods and compositions described herein also modulate other CNS barriers, such as neuronal transport barriers, as well as other CNS barriers.

In some embodiments, the blood brain barrier is modulated with a nitric oxide synthase (NOS) inhibitor. Preferably, the NOS inhibitor is a NOS-3 inhibitor. Non-limiting examples of NOS-3 inhibitors include analogs of L-arginine, such as N^{G}-Monomethyl-L-Arginine (L-NMMA), L-N-Methyl Arginine (L-NMA), N^{G}-Nitro-L-Arginine Methyl Ester (L-NAME), 7-nitroindazole (7-NI). See WO 00/23102, herein incorporated by reference in its entirety.

B. Blood-Brain Barrier Transporters

In some embodiments, the invention provides methods and compositions that modulate ATP Binding Cassette (ABC) transport proteins. ABC transport proteins is a superfamily of membrane transporters with similar structural features. These transport proteins are widely distributed in prokaryotic and eukaryotic cells. They are critical in the maintenance of barrier to foreign molecules and removal of waste from privileged spaces, and may be overexpressed in certain glial tumors conferring drug resistance to cytotoxic drugs. 48 members of the superfamily are described. There are 7 major subfamilies, which include ABC A-G. Subfamilies C, B, and G play a role in transport activity at blood brain barrier and blood-CSF barrier. ABC A substrates include lipids and cholesterol; ABC B transporters include P-glycoprotein (P-gP) and other multi drug resistance proteins (MRPs); ABC C contains MRP proteins; ABC E are expressed in ovary, testis and spleen; and ABC G contains breast cancer resistance protein (BCRP).

Other examples of blood-CSF barrier transporters that can be modulated by methods and compositions of the invention include organic anion transport systems (OAT), P-gP, and the GABA transporters—GAT-1 and GAT2/BGT-1. Substrate compounds for OATs include opiate peptides, including enkephalin and deltorphin II, anionic compounds, indomethacin, salicylic acid and cimetidine. OATs are inhibited by baclofen, tagamet, indomethacin, etc. and transport HVA (dopamine metabolite) and metabolites of norepinephrine, epinephrine, 5-HT3, and histamine.

GABA transporters are Na and Cl dependent, and are specific for GABA, taurine, β alanine, betaine, and nipecotic acid. GAT2 transporters are localized to abluminal and luminal surfaces of capillary endothelial cells. GAT-1 is localized to the outside of neurons and glia. GABA-transporter substrates include lorazepam, midazolam, diazepam, klonazepam and baclofen. Probenicid inhibits luminal membrane GABA transporters from capillary endothelial cells. GAT-1 is inhibited by Tiagabine.

In some embodiments, the invention provides methods and compositions that modulate P-gP, e.g., that activate P-gP. P-gP, also known as ABCB1, forms a protective barrier to pump away by excreting compounds into bile, urine, and intestinal lumen. Three isoforms have been identified in rodents (mdr1a, mdr1b, mdr2) and two in humans (MDR1 and MDR2). It is expressed in epithelium of the brain choroid plexus (which forms the blood-cerebrospinal fluid barrier), as well as on the luminal surface of blood capillaries of the brain (blood-brain barrier) and other tissues known to have blood-tissue barriers, such as the placenta, the ovaries, and the testes.

In the brain, P-gP is expressed in multiple cell types within brain parenchyma including astrocytes and microglia and in luminal plasma membrane of capillary endothelium where it acts as a barrier to entry and efflux pump activity. P-gP transports a wide range of substrates out of cerebral endothelial cells into vascular lumen. P-gP is also expressed in the apical membrane of the choroid plexus and may transport substances into CSF.

P-gP substrates include molecules that tend to be lipophilic, planar molecules or uncharged or positively charged molecules. Non-limiting examples include organic cations, weak organic bases, organic anions and other uncharged compounds, including polypeptides and peptide derivatives, aldosterone, anthracyclines, colchicine, dexamethasone, digoxin, diltiazem, HIV protease inhibitors, loperamide, MTX, morphine, ondansetron, phenyloin and β-blockers. Inhibitors of P-gP include quinidine, verapamil, rifampin, PSC 833 (see Schinkel, J. Clin Invest., 1996, herein incorporated by reference in its entirety) cyclosporine A, carbamazepine, and amitryptiline.

Multi-drug resistance protein (MRP) substrates include acetaminophen glucoronide, protease inhibitors, methotrexate and ampicillin. Inhibitors of MRP include buthionine sulphoximine, an inhibitor of glutathione biosynthesis.

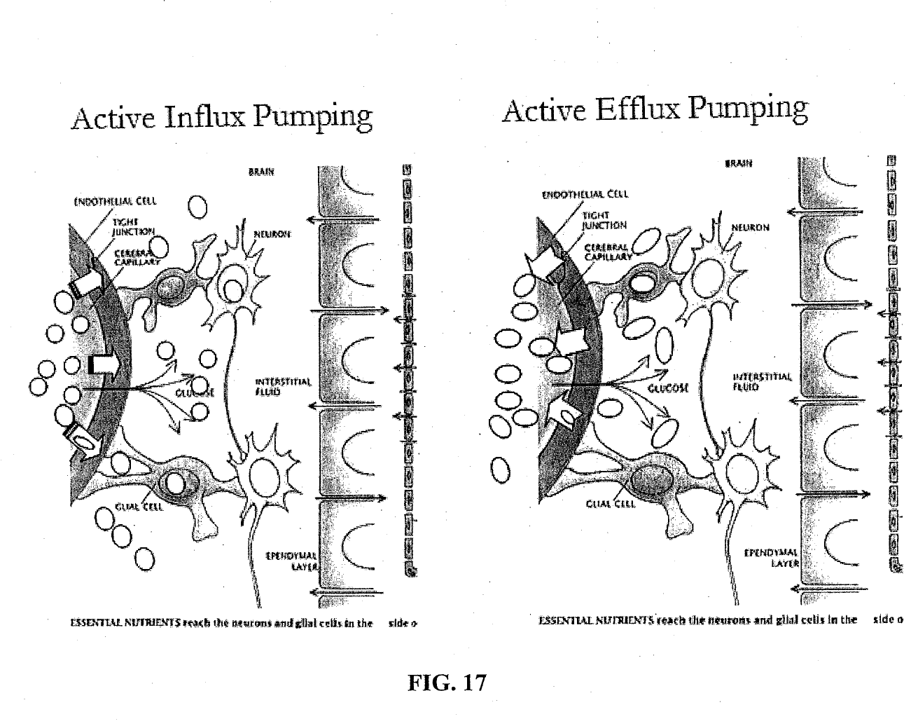

Further information on transporters that can be modulated in embodiments of the methods and compositions of the invention are provided in Table 1 below. FIG. 17 also provides an illustration of active transporters for both influx and efflux.

TABLE 1
Active Transporters in the Blood-Brain Barrier.

| Active Transporter | Physiological Function in Blood-Brain Barrier | Exemplary Substrates |
|---|---|---|
| P-glycoprotein (P-gP) | Limits accumulation in CNS of phospholipids, | Loperamide, morphine, β endorphin, |
| | xenobiotics and other drugs; regulates absorption, | phenytoin, elavil, depakote, cyclosporine, |
| | distribution and elimination of drug substances. | protease inhibitors, digoxin, calcium |
| | | channel blockers, vinca alkaloids, |
| | | anthracyclines, ivermectin, aldosterone, |
| | | hydrocortisone, dexamethasone, taxanes, |
| | | domperidone, ondansetron |
| Multidrug Resistance | MRP family members mediate ATP dependent | Acetaminophen glucoronide, protease |
| (MRP) Protein Family | transport of unconjugated, amphillic anions, and | inhibitors, methotrexate, ampicillin |
| | lipophillic compounds conjugated to glutathione, | |
| | glucoronate, and sulfate; detoxification function | |
| | includes extrusion of leukotriene metabolites; | |
| | folate transport. | |
| GABA transporters (GAT- | GAT1 drives GABA into neurons; mediates | Lorazepam, midazolam, diazepam, |
| 1 and GAT-2, BGT-1) | clearance of GABA from the brain | klonazepam, baclofen |
| Organic Anion Transport | Limits thiopurine uptake; transports HVA | Opiate peptides, including enkephalin and |
| (OAT) Systems | (dopamine metabolite), and metabolites of | deltorphin II, anionic compounds, |
| | norepinephrine, epinephrine, serotonin and | indomethacin, salicylic acid, cimetide |
| | histamine | |

C. Placental Barriers

Access to the fetus from the maternal circulation is controlled by the placenta, a physical barrier that separates the blood supply of the mother and fetus. The major function of the placenta is to transfer nutrients and oxygen from the mother to the fetus and to assist in the removal of waste products from the fetus to the mother. The placenta, therefore, provides a link between the maternal and fetal circulations while simultaneously acting as a barrier to protect the fetus from foreign substances in the maternal blood. Thus, some embodiments of the methods and compositions described herein are for the modulation of access of drugs, therapeutic agents, chemicals and other substances through the placenta. In some embodiments, the methods and compositions involve the modification of the placental barrier to prevent the entry of drugs through the placental barrier and into the fetal environment, e.g., by efflux of drugs across the placenta.

Modulation of the placental barrier to prevent entry of drugs or other foreign substances to the fetal environment is important because of the sensitivity of the fetus to such substances. Studies have shown that nearly all drugs that are administered during pregnancy will enter, to some degree, the circulation of the fetus via passive diffusion, potentially harming the fetus during its growth and developmental stages. See, e.g., Syme, M. R. et al., Clin. Pharmacokinet. 43:487-514 (2004), herein incorporated by reference in its entirety. In addition, the fetus may be additionally harmed by drugs that are actively pumped across the placenta by various transporters located on both the fetal and maternal side of the trophoblast layer. Facilitated diffusion also appears to be a minor transfer mechanism for some drugs. Modulation of the entry pathways through the placenta, therefore, is important to preventing fetal exposure to drugs and other substances present in the maternal circulation.

Placental Development and Anatomy

One of the functions of the placenta, in addition to its barrier-purpose, is to connect the fetus to the uterine wall near the fundus uteri, and more frequently on the posterior than on the anterior wall of the uterus. The placenta during fetal development is formed through the interweaving of both fetal and maternal portions, which allows the close proximity localization of the maternal and fetal circulation systems.

The fetal portion of the placenta consists of the villi of the chorion frondosum. These structures branch repeatedly, and increase in size throughout the fetal developmental stages. The chorion frondosum villi are suspended in the intervillous space where they are bathed in maternal blood. The circulation within the villi are conveyed to the space by the uterine arteries and carried away by the uterine veins. A branch of an umbilical artery enters each villus and ends in a capillary plexus from which the blood is drained by a tributary of the umbilical vein. The vessels of the villus are surrounded by a thin layer of mesoderm consisting of gelatinous connective tissue, which is covered by two strata of ectodermal cells derived from the trophoblast: the deeper stratum. The next layer of tissue consists of the mesodermic tissue, which represents the cytotrophoblast or layer of Langhans. The superficial layer, which is in contact with the maternal blood, is the syncytiotrophoblast. After the fifth month, the two strata of cells are replaced by a single layer of flattened cells.

The maternal portion of the placenta is formed by the decidua placentalis containing the intervillous space. As mentioned above, this space is produced by the enlargement and intercommunication of the spaces in the trophoblastic network. The changes involve the disappearance of the greater portion of the stratum compactum, but the deeper part of this layer persists and is condensed to form what is known as the basal plate. Between the basal plate and the uterine muscular fibers are the stratum spongiosum and the boundary layer. Through the stratum spongiosum, boundary layer and the basal plate, the uterine arteries and veins pass to and from the intervillous space. The endothelial lining of the uterine vessels ceases at the point where they terminate in the intervillous space, which is lined by the syncytiotrophoblast. Portions of the stratum compactum persist and are condensed to form a series of septa, which extend from the basal plate through the thickness of the placenta and subdivide it into the lobules or cotyledons seen on the uterine surface of the detached placenta. The cotyledons function as a vascular unit within the placenta.

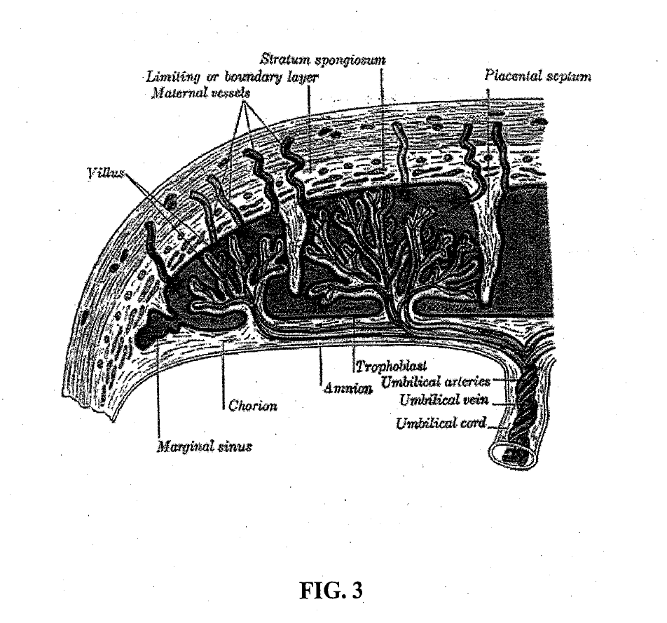

The fetal and maternal blood currents traverse the placenta, the former passing through the blood vessels of the placental villi and the latter through the intervillous space (see FIG. 3). The two circulations do not intermingle, being separated from each other by the delicate walls of the villi. Nevertheless, the fetal blood is able to absorb, through the walls of the villi, oxygen and nutritive materials from the maternal blood, and give up to the latter its waste products. The purified blood is carried back to the fetus by the umbilical vein. The placenta, therefore, not only establishes a mechanical connection between the mother and the fetus, but also provides nutrition, respiration, and excretion services for the fetus.

During embryonic and early fetal development, the maternal blood does not communicate with the fetal circulation through the placenta. Maternal blood does not perfuse the placenta during the embryonic period and the feto-placental-maternal circulation does not become established until around the tenth week of pregnancy. Hence, access of drugs and other chemicals present in the maternal blood during the first 10 weeks of gestation occurs via diffusion through extracellular fluid. Maternal blood access to the placental circulation only occurs after development and establishment of the feto-placental-maternal circulation.

D. Placental Transport Mechanisms

Transplacental exchanges are known to involve passive transfer, active transport, facilitated diffusion, phagocytosis and pinocytosis. See, e.g., Pacifici G M, et al., Clin. Pharmacokinet. 28:235-69 (1995), herein incorporated by reference. Studies, however, have shown that phagocytotic and pinocytotic mechanisms are too slow to have any significant influence on drug or chemical transfer from the maternal circulation to the fetus. Syme et al. (2004). Therefore, one embodiment of the methods and compositions disclosed herein is to modulate passive transfer, facilitated diffusion and active transport of drugs, therapeutic agents, chemicals and other substances across the placental barrier.

Passive Transfer

One embodiment is the modulation of passive transfer of drugs, chemicals and other substances across the placental barrier. Passive transfer represents the permeation of a molecule through a physical barrier, such as a cell membrane, down its concentration gradient. Passive diffusion does not require the input of energy, is not saturable and is not subject to competitive inhibition. When drugs cross the placenta by passive diffusion, the amount that crosses in any given time is dependent on the concentration of the drug in the maternal circulation, its physicochemical properties and the properties of the placenta that determine how readily the drug will pass.

Passive diffusion is favored for low-molecular weight and highly lipid-soluble drugs that are predominantly un-ionized. The placenta resembles a lipid bilayer membrane, so only the non-protein bound portion of a drug, barring any applicable active-transport mechanisms, is free to diffuse across it.

Facilitated Diffusion

Another embodiment of the methods and compositions disclosed herein is the modulation of facilitated diffusion mechanisms in the placental barrier. Facilitated diffusion requires the presence of a carrier substance within the placenta. Moreover, the transport of the system becomes saturated at high concentrations relative to the Michaelis-Menten constant (Kₘ) of the transporter. However, transport by this mechanism does not require the input of energy, as opposed to active transport of substances. Facilitated diffusion usually equalizes the concentration of drugs, chemicals, or substances between the maternal and fetal circulations. It may be that for many substances, such as carbohydrates, facilitated diffusion provides a means to increase transport rates when the functional and metabolic needs of the fetus would not be met by passive diffusion alone. Folkart G R, et al. Am. J. Obstet. Gynecol., 80:221-223 (1960), herein incorporated by reference.

Studies have shown that only a few drugs use facilitated diffusion mechanisms to traverse the placental barrier. Ganciclovir has been demonstrated to be taken up into maternal-facing syncytiotrophoblast vesicles by a carrier-dependent system. Henderson G I et al., Am. J. Med. Sci. 306:151-156 (1993). However, transport of Ganciclovir probably involves a combination of passive and facilitated diffusion mechanisms, the rate-limiting transfer step being passive diffusion. Syme et al. (2004). Placental carrier-mediated transport systems have also been found in maternal-facing syncytiotrophoblast membrane vesicles for cephalosporin, cephalexin and glucocorticoids. Kudo Y, et al., Biochim. Biophys. Acta 731:415-420 (1989); Fant M E, et al., Biochim. Biophys. Acta 731:415-420 (1983), incorporated by reference herein. In light of the relatively few drugs that use this mechanism, it has been suggested that structurally related endogenous compounds, such as hormones and nucleosides, will most likely be the primary species to benefit from this transport system. Syme et al. (2004).

Active Transporters

Another embodiment of the methods and compositions disclosed herein is use of modulators or therapeutic agents in manipulating active transport of drugs, chemicals and other substances across the placental barrier. Active transport across the placental barrier, as opposed to facilitated diffusion or passive transport, requires energy, usually in the form of adenosine triphosphate (ATP) or through energy stored in the transmembrane electrochemical gradient provided by Na⁺, Cl⁻ or H⁺. Because of the input of energy, active transport systems may work against a concentration gradient, however, saturation of the transporters can occur.

Extensive studies have been conducted regarding placental transport systems of nutrients, such as amino acids, vitamins and glucose. See Hahn T, et al., Early Pregnancy 2:168-182 (1996); Moe A J, Am. J. Physiol. 268:C1321-1331 (1995); Bissonnette J M, Mead Johnson Symp. Perinat. Dev. Med., 18:21-23 (1981), all incorporated herein by reference. Active transport of drugs occurs through the same transport systems, most likely due to structurally similarities between the transported drugs and endogenous substrates. Syme et al. (2004).

Active drug transporters are located either in the maternal-facing brush border (apical) membrane or the fetal-facing basolateral (basal) membrane where they pump drugs into or out of the synctiotrophoblast. Table 2 summarizes the active transporters that have been identified in the placenta.

TABLE 2
Active transporters in Placenta.

| Active Transporter | Physiological Function in Placenta | Exemplary Substrates |
|---|---|---|
| P-glycoprotein (P-gP) | Fetal-to-maternal transfer of hydrophobic | Digoxin, cyclosporine, saquinavir, |
| | cationic compounds | vincristine, vinblastine, paclitaxel, |
| | | dexamethasone, terfenadine, |
| | | sirolimus, quinidine, ondansetron, |
| | | loperamide |
| Multidrug resistance protein | Fetal-to-maternal transfer of glutathione, | Methotrexate, etoposide, vincristine, |
| 1 (MRP1) | sulfate and glucoronide conjugates (dianionic | cisplatin, vinblastine, HIV protease |
| | sulfated bile salts) | inhibitors |
| Multidrug resistance protein | Fetal-to-maternal transfer of glutathione, | Etoposide, cisplatin, doxorubicin, |
| 2 (MRP2) | sulfate and glucoronide conjugates (dianionic | vincristine, vinblastine, methotrexate, |
| | sulfated bile salts, bilirubin glucoronide, | paracetamol, glucoronide, |
| | estradiol glucoronide) | grepafloxacin, ampilicillin |
| Multidrug resistance protein | Fetal-to-maternal transfer of anionic | Methotrexate, etoposide |
| 3 (MRP3) | conjugates | |
| Breast cancer resistant | Unknown | Topotecan, mitoxantrone, |
| protein (BCRP) | | doxorubicin, daunorubicin |
| Serotonin transporter (SERT) | Serotonin transfer | Amphetamines |
| Norepinephrine transporter | Dopamine and norepinephrine transfer | Amphetamines |
| (NET) | | |
| Extraneuronal monoamine | Serotonin, dopamine, norepinephrine, | Amphetamines, imipramine, |
| transporter (OCT3) | histamine transfer | desipramine, clonidine, cimetidine |
| Organic cation transporters | Maternal-to-fetal transfer of carnitine | Metamphetamine, quinidine, |
| (OCTN) | | verapamil, pyrilamine |
| Monocarboxylate | Fetal-to-maternal transfer of lactate and | Valproic acid |
| transporters | pyruvate | |
| Dicarboxylate transporters | Maternal-to-fetal transfer of succinate and α- | Unknown |
| | ketoglutarate | |
| Sodium/multivitamin | Maternal-to-fetal transfer of biotin and | Carbamazepine, primidone |
| transporter (SMVT) | pantothenate | |

P-Glycoproteins (P-gP)

Another embodiment of the methods and compositions disclosed herein is the modulation of the placental P-gP transporter. The multidrug resistant gene (MDR1) product, P-glycoprotein, is a member of the ATP-binding cassette (ABC) transporter family. In the placenta, P-gP is expressed in the trophoblast cells of the brush-border membrane, but not the basal membrane. Cordon-Cardo C. et al., J. Histochem. Cytochem. 38:1277-87 (1990); Sugawara I, et al., Cancer Res. 48:1926-1929 (1988), herein incorporated by reference in its entirety. Studies have demonstrated that placental P-gP regulates the transfer of cyclosporine, vincristine, vinblastine and digoxin into trophoblast cells. Ushigome F, et al., Eur. J. Pharmacol. 408:1-10 (2000); Pavek P, et al., J. Pharm. Sci. 10: 1583-1592 (2001), herein incorporated by reference. However, the transfer of the drugs were predominantly in the fetal-to-maternal transfer direction, thereby reducing fetal exposure to the drugs. Ushigame et al. (2000).

Studies in the mdr1a (P-gP) knockout (−/−) mouse demonstrate the importance of the P-gP transporter in reducing fetal exposure to drugs and other chemicals or substances. For example, Lankas et al. (Reprod. Toxicol. 12:457-463 (1998), herein incorporated by reference) has shown that administration of an isomer of the pesticide avermectin was associated with a 100% incidence of fetal cleft palate in the mdr1a knockout mice. In contrast, heterozygous (+/−) mice were less sensitive and homozygous (+/+) mice insensitive at the same doses tested on the knockout mice. In addition, the degree of chemical exposure was inversely related to the expression of P-gP, which was determined by fetal genotyping. Other studies in mdr1a knockout mice have confirmed the major fetoprotective role that the P-gP transporter plays. Smit J W, et al., J. Clin. Invest. 104:1441-1447 (1999).

Multidrug Resistance Associated Protein (MRP) Family

Another embodiment of the methods and compositions disclosed herein is the modulation of placental MRP transporters. The MRP family consists of seven members, designated MRP1 to MRP7. For review, see Borst P, et al., J. Natl. Cancer Inst. 92:1295-1302 (2000), herein incorporated by reference. In human placenta, at least three members of the MRP family have been identified: MRP1, MRP2 and MRP3. Sugawara I, et al., Cancer Lett. 112:23-31 (1997); St-Pierre V, et al., Am. J. Physiol. Regul. Integr. Comp. Physiol. 279:R1495-1503 (2000); Flens M J et al., Am. J. Pathol. 148:1237-1247 (1996), herein incorporated by reference. MRP 1 and MRP 3 were found to be localized primarily in the fetal endothelial cells of the placenta microcapillary. Hipfner D R, et al., Biochim. Biophys. Acta 1461:359-376 (1999). MRP2, MRP3, and to a lesser extent MRP1, are also expressed in the apical membrane of the synctiotrophoblast. Sugawara et al. (1997); Flens et al. (1996) and St.-Pierre et al. (2000).

MRP-related placental proteins transport a variety of substrates primarily in the direction of the fetal-to-maternal transfer. Accordingly, researchers have suggested that MRP-transporters could exert a feto-protective role by the removal of metabolic end products from the fetus to the mother. St.-Pierre et al. (2000); Cui Y, et al., Mol. Pharmacol. 55:929-937 (1999), herein incorporated by reference.

Breast Cancer Resistant Protein (BCRP)

Another embodiment of the methods and compositions disclosed herein is the modulation of placental BCRP transporters. BCRP, an ATP-driven transporter, is highly expressed in the placenta. Allikmets R., et al., Cancer Res. 58:5337-5339 (1998), herein incorporated by reference. BCRP is responsible for rendering tumor cells resistant to chemotherapeutic agents, such as topotecan, mitoxantrone, doxorubicin and daunorubicin. Allen J D, et al., Cancer Res. 59:4237-4241 (1999). BCRP has also been shown to restrict the passage of topotecan and mitoxantrone to the fetus in mice. Jonker J W et al., J. Natl. Cancer Inst. 92:1651-1656 (2000), herein incorporated by reference.

Monoamine Transporters

Yet another embodiment is the modulation of monoamine transporters in placenta. Studies have identified the placental monoamine transporters as serotonin transporter (SERT), norepinephrine transporter (NET) and the extraneuronal monoamine transporter (OCT3). Ramamoorthy S, et al., Placenta 14:449-461 (1993); Ramamoorthy S., et al., Biochem. 32:1346-1353 (1993); Kekuda R., et al., J. Biol. Chem. 273:15971-15979 (1998), all herein incorporated by reference. SERT and NET derive energy from the transmembrane Na⁺ and Cl⁻ electrochemical gradient, and are primarily localized in the brush-border membrane of the placental trophoblast. Both SERT and NET transport serotonin, dopamine and norepinephrine from the maternal circulation to the fetus. Drug substrates of the SERT and NET transporters include amphetamines, although cocaine and non-tricyclic antidepressants bind to the SERT and NET transporters with high affinity without being transferred across the membrane.

OCT3 is localized to the basal membrane, where it transports serotonin, dopamine, norepinephrine and histamine via a Na⁺ and Cl⁻ independent system. Ganaphthy V et al., J. Pharmacol. Exp. Ther. 294:413-420 (2000); Kekuda et al. (1998). Amphetamines, imipramine and desipramine may be actively transported by placental OCT3.

Organic Cation Transporters

One additional embodiment of the present invention is the modulation of placental Organic Cation Transporters. Placental Na+-driven organic cation transporter 2 (OCTN2) has been identified and localized to the basal membrane of the synctiotrophoblast. Wu X et al., J. Pharmacol. Exp. Ther. 290:1482-1492 (1999), herein incorporated by reference. Placental OCTN2 transports carnitine across the placenta in the direction of the maternal-to-fetal transfer. Ohashi R., et al., J. Pharmacol. Exp. Ther. 291:778-784 (1999), herein incorporated by reference. Studies have identified metamphetamine, quinidine, verapamil, pyrilamine, desipramine, dimethylamiloride, cimetidine, and procainimide as drug substrates for OCTN2. Wu X, et al., Biochem. Biophys. Res. Commun. 246:589-595 (1998); Wu X, et al., Biochim. Biophys. Acta 1466:315-327 (2000), herein incorporated by reference.

Monocarboxylate Transporters and the Dicarboxylate Transporters

Another embodiment of the methods and compositions disclosed herein is the modulation of monocarboxylate (MCT) and dicarboxylate (NaDC3 transporters. Both MCT (e.g. lactate transport) and NaDC3 (e.g. succinate transport), which utilize electrochemical gradients for transport, are localized to the brush border membrane of the placenta, with MCT being expressed in the basal membrane to a lesser extent. Price N T, et al., Biochem. J. 329:321-328 (1998); Ganaphthy V, et al., Biochem J. 249:179-184 (1988); Balkovetz D F, et al., 263:13823-13830 (1988), all incorporated by reference herein. Valproic acid, a teratogenic substance, may be a substrate for MCT transfer, and compete with lactate for transport across the placental barrier. Nakamura H. et al., Pharm. Res. 19:154-161 (2002), herein incorporated by reference.

III Transporter Modulators (e.g., Activators or Inhibitors)

The invention provides compositions and methods for reducing or eliminating the effects of a substance in the CNS and/or in the fetus. In some embodiments, the compositions and embodiments described herein modulate the efflux of drugs or other compounds out of physiological compartments, including across the blood brain barrier and/or placental barrier via a BBB or fetal transport protein, e.g., the P-gP transporter. In some embodiments, such modulators activate and/or increase the efflux by the BBB or fetal transport protein, e.g., P-gP transporters on the blood brain barrier and/or placental barriers.

Modulators may be any suitable modulator. In some embodiments, modulators useful in the invention are polyphenols, such as flavonoids. Suitable modulators include catechins from green tea, including (−) epicatechin. See Wang, E, et al., Biochem. Biophys. Res. Comm. 297:412-418 (2002); Zhou, S., et al., Drug Metabol. Rev. 36:57-104 (2004), both of which are herein incorporated by reference in its entirety. Other suitable modulators, e.g., P-gP modulators for use herein include flavonols, including, but not limited to, kaempferol, quercetin, and galangin.

In other embodiments, P-gP transporter modulators may include small molecules, including 2-p-Tolyl-5,6,7,8-tetrahydrobenzo[d]imidazo[2,1-b]thiazole; 1-Carbazol-9-yl-3-(3,5-dimethylpyrazol-1-yl)-propan-2-ol; 2-(4-Chloro-3,5-dimethylphenoxy)-N-(2-phenyl-2H-benzotriazol-5-yl)-acetamide; N-[2-(4-Chloro-phenyl)-acetyl]-N′-(4,7-dimethyl-quinazolin-2-yl)-guanidine; 1-Benzyl-7,8-dimethoxy-3-phenyl-³H-pyrazolo[3,4-c]isoquinoline; N-(3-Benzooxazol-2-yl-4-hydroxyphenyl)-2-p-tolyloxyacetamide; 8-Allyl-2-phenyl-⁸H-1,3a,8-triazacyclopenta[a]indene; 3-(4-Chloro-benzyl)-5-(2-methoxyphenyl)-[1,2,4]oxadiazole; 2-Phenethylsulfanyl-5,6,7,8-tetrahydrobenzo[4,5]thieno[2,3-d]pyrimidin-4-ylamine; (5,12,13-Triaza-indeno[1,2-b]anthracen-13-yl)-acetic acid ethyl ester; 2,2′-(1-phenyl-¹H-1,2,4-triazole-3,5-diyl)bis-phenol; and 2-(2-Chloro-phenyl)-5-(5-methylthiophen-2-yl)-[1,3,4]oxadiazole. See Kondratov, et al., Proc. Natl. Acad. Sci. 98:14078-14083 (2001), herein incorporated by reference in its entirety.

In one embodiment, a P-gP substrate is used to inhibit transport across the blood brain barrier and/or the placenta. Multi Drug Resistance Proteins consist of a family of plasma membrane proteins encoded by the MDR (multidrug resistance) gene. The most well characterized member of this family, P-glycoprotein (P-gP) functions as a membrane-localized drug efflux transport mechanism that has the ability to actively pump away many drug substrates (including all currently prescribed HIV-protease inhibitors and many anti-cancer agents) from the intracellular cytoplasm, substantially attenuating their localized effects. The clinical effect of P-gP efflux activity on a HIV-protease inhibitor is a decrease of drug concentration in the brain, which can render drug therapy inconsistent and unsuccessful. However, if the goal of said drug administration is to achieve a localized effect, restrict bioavailability, and reduce CNS (or other tissue) exposure, administration of a compound with P-gP affinity (“substrate”) would be beneficial when incorporated into a drug formulation.

In some embodiments, the invention utilizes a modulator of a BBB transport protein. In some embodiments, the invention utilizes a modulator of a BBB transport protein that is an ABC transport protein. In some embodiments, the invention utilizes a BBB transport protein activator. In some embodiments, the BBB transport protein modulator is a modulator of P-gP, e.g., an activator of P-gP.

One class of compounds useful in the compositions and methods of the invention is polyphenols. Many polyphenols are modulators of BBB transport proteins; however, any suitable polyphenol that produces a decrease of one or more CNS effects of a substance, no matter what the mechanism, may be used in the compositions and methods of the invention.

A particularly useful class of polyphenols is the flavonoids. Flavonoids, the most abundant polyphenols in the diet, can be classified into subgroups based on differences in their chemical structures. The basic flavonoid structure is shown below (formula I):

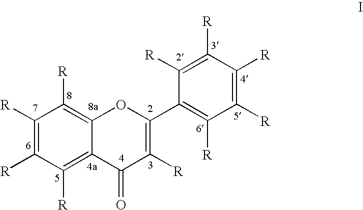

wherein the 2,3 bond may be saturated or unsaturated, and wherein each R can be independently selected from the group consisting of hydrogen, substituted or unsubstituted hydroxyl, substituted or unsubstituted amine, substituted or unsubstituted thiol, substituted or unsubstituted C₁-C₁₀ alkyl, substituted or unsubstituted C₁-C₁₀ alkynyl, substituted or unsubstituted C₁-C₁₀ alkenyl, substituted or unsubstituted aryl, substituted or unsubstituted heteroaryl, substituted or unsubstituted C₅-C₁₀ cycloalkyl, substituted or unsubstituted C₅-C₁₀ heterocycloalkyl, substituted or unsubstituted C₁-C₁₀ aliphatic acyl, substituted or unsubstituted C₁-C₁₀ aromatic acyl, trialkyl silyl, substituted or unsubstituted ether, carbohydrate, and substituted carbohydrate;

and its pharmaceutically acceptable salts, esters, prodrugs, analogs, isomers, stereoisomers or tautomers thereof.

“Carbohydrate” as used herein, includes, but not limited to, monosaccharides, disaccharides, oligosaccharides, or polysaccharides. Monosaccharide for example includes, but not limited to, allose, altrose, mannose, gulose, Idose, glucose, galactose, talose, and fructose. Disaccharides for example includes, but not limited to, glucorhamnose, trehalose, sucrose, lactose, maltose, galactosucrose, N-acetyllactosamine, cellobiose, gentiobiose, isomaltose, melibiose, primeverose, hesperodinose, and rutinose. Oligosaccharides for example includes, but not limited to, raffinose, nystose, panose, cellotriose, maltotriose, maltotetraose, xylobiose, galactotetraose, isopanose, cyclodextrin (α-CD) or cyclomaltohexaose, β-cyclodextrin (β-CD) or cyclomaltoheptaose and γ-cyclodextrin (γ-CD) or cyclomaltooctaose. Polysaccharide for example includes, but not limited to, xylan, mannan, galactan, glucan, arabinan, pustulan, gellan, guaran, xanthan, and hyaluronan. Some examples include, but not limited to, starch, glycogen, cellulose, insulin, chitin, amylose and amylopectin.

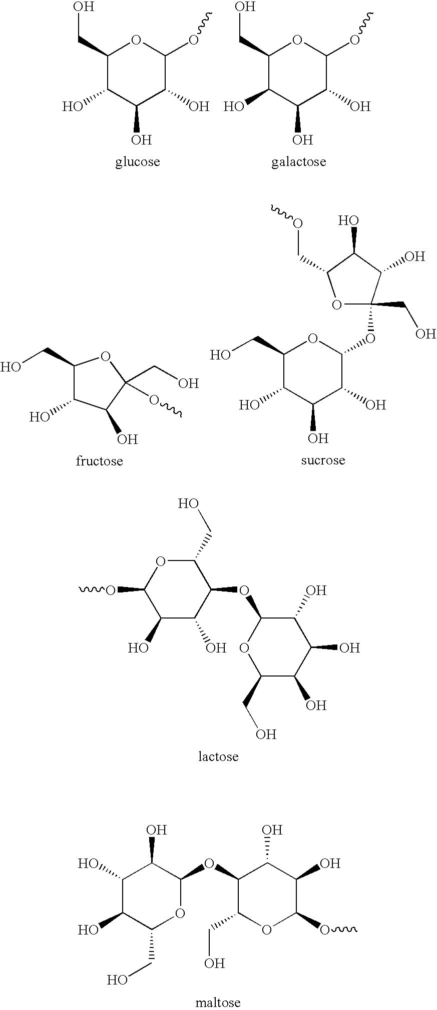

In some embodiments, the invention utilizes a flavonoid where the molecule is planar. In some embodiments, the invention utilizes a flavonoid where the 2-3 bond is unsaturated. In some embodiments, the invention utilizes a flavonoid where the 3-position is hydroxylated. In some embodiments, the invention utilizes a flavonoid where the 2-3 bond is unsaturated and the 3-position is hydroxylated (e.g., flavonols).

In some embodiments, the invention utilizes one or more flavonoids selected from the group consisting of quercetin, isoquercetin, flavone, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments, the invention utilizes one or more flavonoids selected from the group consisting of quercetin, isoquercetin, apigenin, rhoifolin, galangin, fisetin, morin, rutin, kaempferol, myricetin, naringenin, hesperetin, phloretin, and genistein. Structures of these compounds are well-known in the art. See, e.g., Critchfield et al. (1994) Biochem. Pharmacol 7:1437-1445.

In some embodiments, the invention utilizes a flavonol. In some embodiments, the flavonol is selected from the group consisting of quercetin, fisetin, morin, rutin, myricetin, galangin, and kaempherol, and combinations thereof. In some embodiments, the flavonol is selected from the group consisting of quercetin, galangin, and kaempherol, and combinations thereof. In some embodiments, the flavonol is quercetin. In some embodiments, the flavonol is galangin. In some embodiments, the flavonol is kaempherol.

A particularly useful flavonol is quercetin. Quercetin may be used to illustrate formulations and methods useful in the invention, however, it is understood that the discussion of quercetin applies equally to other flavonoids, flavonols, and polyphenols useful in the invention, e.g., kaempferol and galangin.

The structure of quercetin is shown below (formula II):

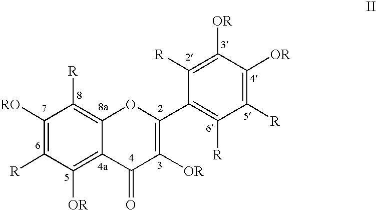

wherein each OR is an OH (i.e., 3-OH, 5-OH, 7-OH, 3′-OH, and 4′-OH) and each R is an H. The numbering of the carbons is the same as in Formula I. This form of quercetin is used in some embodiments of the invention. As used herein, the term “quercetin” also encompasses derivatives of quercetin, wherein each R can be independently selected from the group consisting of hydrogen, substituted or unsubstituted C₁-C₁₀ alkyl, substituted or unsubstituted aryl, substituted or unsubstituted C₁-C₁₀ aliphatic acyl, substituted or unsubstituted C₁-C₁₀ aromatic acyl, trialkyl silyl, substituted or unsubstituted ether, carbohydrate, and substituted carbohydrate;

and its pharmaceutically acceptable salts, esters, prodrugs, analogs, isomers, stereoisomers or tautomers thereof. In addition, metabolites of quercetin, e.g., quercetin 3-O-glucoronide, are encompassed by the term “quercetin” as used herein.

In some embodiments, the quercetin is in a carbohydrate-derivatized form, e.g., a quercetin-O-saccharide. Quercetin-O-saccharides useful in the invention include, but are not limited to, quercetin 3-O-glycoside, quercetin 3-O-glucorhamnoside, quercetin 3-O-galactoside, quercetin 3-O-xyloside, and quercetin 3-O-rhamnoside. In some embodiments, the invention utilizes a quercetin 7-O-saccharide.

In some embodiments, the invention utilizes a quercetin aglycone. In some embodiments, a combination of aglycones and carbohydrate-derivatized quercetins is used. It will be appreciated that the various forms of quercetin may have different properties useful in the compositions and methods of the invention, and that the route of administration can determine the choice of forms, or combinations of forms, used in the composition or method. Choice of a single form, or of combinations, is a matter of routine experimentation.

Thus, in some embodiments the invention features a composition or method utilizing quercetin to reduce or eliminate one or more CNS or fetal effects of a substance, such as a therapeutic agent, e.g., an analgesic.

In some embodiments, the quercetin is provided in a form for oral consumption. Oral bioavailability of quercetin O-saccharides is generally superior to that of quercetin aglycones. The bioavailability of the various components is dependent on 1) the site of carbohydrate moiety or moieties and ii) the pendant sugar unit. In addition it is believed that specific carriers are responsible for the absorption of various quercetin glycosides, as well as specific intestinal betaglucosidases. After distribution in the body, the major metabolite, quercetin glucuronide (e.g., quercetin 3-O-glucouronid), is found. Oral bioavailability is sensitive to the presence of food factors.

In compositions for oral delivery of quercetin, carbohydrate-derivatized forms (also referred to herein as “quercetin saccharides”) are used in some embodiments. In some embodiments, quercetin-3-O-glycoside is used in an oral preparation of quercetin; in some embodiments, a pharmaceutically acceptable excipient is included in the composition. In some embodiments, quercetin 3-O-glucorhamnoside is used in an oral preparation of quercetin; in some embodiments, a pharmaceutically acceptable excipient is included in the composition. In some embodiments, a combination of quercetin-3-O-glycoside and quercetin 3-O-glucorhamnoside is used in an oral preparation of quercetin; in some embodiments, a pharmaceutically acceptable excipient is included in the composition. Other carbohydrate-derivatized forms of quercetin, or other forms of quercetin which are derivatives as described above, can also be used, based on their oral bioavailability, their metabolism, their incidence of gastrointestinal or other side effects, and other factors known in the art. Determining the bioavailability of quercetin in the form of derivatives including aglycones and glycosides is a matter of routine experimentation. See, e.g., Graefe et al., J. Clin. Pharmacol. (2001) 451:492-499; Arts et al. (2004) Brit. J. Nutr. 91:841-847; Moon et al. (2001) Free Rad. Biol. Med. 30:1274-1285; Hollman et al. (1995) Am. J. Clin. Nutr. 62:1276-1282; Jenaelle et al. (2005) Nutr. J. 4:1, and Cermak et al. (2003) J. Nutr. 133: 2802-2807, all of which are incorporated by reference herein in their entirety.

In some embodiments, the invention provides a composition for administration of quercetin to an animal to reduce a CNS effect of a substance, e.g., for the oral delivery of quercetin, that contain at least about 1, 5, 10, 20, 30, 40, 50, 60, 70, 80, 90, 95, 99, 99.5, 99.9, or 99.99% quercetin-O-saccharide. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-O-saccharide. In some embodiments, the invention provides a composition that contains about 1-100% quercetin-O-saccharide, or about 10-100% quercetin-O-saccharide, or about 20-100% quercetin-O-saccharide, or about 50-100% quercetin-O-saccharide, or about 80-100% quercetin-O-saccharide, or about 90-100% quercetin-O-saccharide, or about 95-100% quercetin-O-saccharide, or about 99-100% quercetin-O-saccharide. In some embodiments, the invention provides a composition that contains about 1-90% quercetin-O-saccharide, or about 10-90% quercetin-O-saccharide, or about 20-90% quercetin-O-saccharide, or about 50-90% quercetin-O-saccharide, or about 80-90% quercetin-O-saccharide. In some embodiments, the invention provides a composition that contains about 1-75% quercetin-O-saccharide, or about 10-75% quercetin-O-saccharide, or about 20-75% quercetin-O-saccharide, or about 50-75% quercetin-O-saccharide. In some embodiments, the invention provides a composition that contains about 1-50% quercetin-O-saccharide, or about 10-50% quercetin-O-saccharide, or about 20-50% quercetin-O-saccharide, or about 30-50% quercetin-O-saccharide, or about 40-50% quercetin-O-saccharide. In some embodiments, the invention provides a composition that contains about 1-40% quercetin-O-saccharide, or about 10-40% quercetin-O-saccharide, or about 20-40% quercetin-O-saccharide, or about 30-40% quercetin-O-saccharide. In some embodiments, the invention provides a composition that contains about 1-30% quercetin-O-saccharide, or about 10-30% quercetin-O-saccharide, or about 20-30% quercetin-O-saccharide. In some embodiments, the invention provides a composition that contains about 1-20% quercetin-O-saccharide, or about 10-20% quercetin-O-saccharide. In some embodiments, the invention provides a composition that contains about 1-10% quercetin-O-saccharide. In some embodiments, the invention provides a composition that contains about 1, 2, 5, 10, 20, 30, 40, 50, 60, 70, 80, 90, 95, 96, 97, 98, or 99% quercetin-O-saccharide.

In some embodiments, the invention provides a composition for administration of quercetin to an animal to reduce a CNS effect of a substance, e.g., for the oral delivery of quercetin, that contain at least about 1, 5, 10, 20, 30, 40, 50, 60, 70, 80, 90, 95, 99, 99.5, 99.9, or 99.99% quercetin-3-O-glycoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glycoside. In some embodiments, the invention provides a composition that contains about 1-100% quercetin-3-O-glycoside, or about 10-100% quercetin-3-O-glycoside, or about 20-100% quercetin-3-O-glycoside, or about 50-100% quercetin-3-O-glycoside, or about 80-100% quercetin-3-O-glycoside, or about 90-100% quercetin-3-O-glycoside, or about 95-100% quercetin-3-O-glycoside, or about 99-100% quercetin-3-O-glycoside. In some embodiments, the invention provides a composition that contains about 1-90% quercetin-3-O-glycoside, or about 10-90% quercetin-3-O-glycoside, or about 20-90% quercetin-3-O-glycoside, or about 50-90% quercetin-3-O-glycoside, or about 80-90% quercetin-3-O-glycoside. In some embodiments, the invention provides a composition that contains about 1-75% quercetin-3-O-glycoside, or about 10-75% quercetin-3-O-glycoside, or about 20-75% quercetin-3-O-glycoside, or about 50-75% quercetin-3-O-glycoside. In some embodiments, the invention provides a composition that contains about 1-50% quercetin-3-O-glycoside, or about 10-50% quercetin-3-O-glycoside, or about 20-50% quercetin-3-O-glycoside, or about 30-50% quercetin-3-O-glycoside, or about 40-50% quercetin-3-O-glycoside. In some embodiments, the invention provides a composition that contains about 1-40% quercetin-3-O-glycoside, or about 10-40% quercetin-3-O-glycoside, or about 20-40% quercetin-3-O-glycoside, or about 30-40% quercetin-3-O-glycoside. In some embodiments, the invention provides a composition that contains about 1-30% quercetin-3-O-glycoside, or about 10-30% quercetin-3-O-glycoside, or about 20-30% quercetin-3-O-glycoside. In some embodiments, the invention provides a composition that contains about 1-20% quercetin-3-O-glycoside, or about 10-20% quercetin-3-O-glycoside. In some embodiments, the invention provides a composition that contains about 1-10% quercetin-3-O-glycoside. In some embodiments, the invention provides a composition that contains about 1, 2, 5, 10, 20, 30, 40, 50, 60, 70, 80, 90, 95, 96, 97, 98, or 99% quercetin-3-O-glycoside.

In some embodiments, the invention provides a composition for administration of quercetin to an animal to reduce a CNS effect of a substance, e.g., for the oral delivery of quercetin, that contain at least about 1, 5, 10, 20, 30, 40, 50, 60, 70, 80, 90, 95, 99, 99.5, 99.9, or 99.99% quercetin-3-O-glucorhamnoside. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin-3-O-glucorhamnoside. In some embodiments, the invention provides a composition that contains about 1-100% quercetin-3-O-glucorhamnoside, or about 10-100% quercetin-3-O-glucorhamnoside, or about 20-100% quercetin-3-O-glucorhamnoside, or about 50-100% quercetin-3-O-glucorhamnoside, or about 80-100% quercetin-3-O-glucorhamnoside, or about 90-100% quercetin-3-O-glucorhamnoside, or about 95-100% quercetin-3-O-glucorhamnoside, or about 99-100% quercetin-3-O-glucorhamnoside. In some embodiments, the invention provides a composition that contains about 1-90% quercetin-3-O-glucorhamnoside, or about 10-90% quercetin-3-O-glucorhamnoside, or about 20-90% quercetin-3-O-glucorhamnoside, or about 50-90% quercetin-3-O-glucorhamnoside, or about 80-90% quercetin-3-O-glucorhamnoside. In some embodiments, the invention provides a composition that contains about 1-75% quercetin-3-O-glucorhamnoside, or about 10-75% quercetin-3-O-glucorhamnoside, or about 20-75% quercetin-3-O-glucorhamnoside, or about 50-75% quercetin-3-O-glucorhamnoside. In some embodiments, the invention provides a composition that contains about 1-50% quercetin-3-O-glucorhamnoside, or about 10-50% quercetin-3-O-glucorhamnoside, or about 20-50% quercetin-3-O-glucorhamnoside, or about 30-50% quercetin-3-O-glucorhamnoside, or about 40-50% quercetin-3-O-glucorhamnoside. In some embodiments, the invention provides a composition that contains about 1-40% quercetin-3-O-glucorhamnoside, or about 10-40% quercetin-3-O-glucorhamnoside, or about 20-40% quercetin-3-O-glucorhamnoside, or about 30-40% quercetin-3-O-glucorhamnoside. In some embodiments, the invention provides a composition that contains about 1-30% quercetin-3-O-glucorhamnoside, or about 10-30% quercetin-3-O-glucorhamnoside, or about 20-30% quercetin-3-O-glucorhamnoside. In some embodiments, the invention provides a composition that contains about 1-20% quercetin-3-O-glucorhamnoside, or about 10-20% quercetin-3-O-glucorhamnoside. In some embodiments, the invention provides a composition that contains about 1-10% quercetin-3-O-glucorhamnoside. In some embodiments, the invention provides a composition that contains about 1, 2, 5, 10, 20, 30, 40, 50, 60, 70, 80, 90, 95, 96, 97, 98, or 99% quercetin-3-O-glucorhamnoside.

In some embodiments, the invention provides a composition for administration of quercetin to an animal to reduce a CNS effect of a substance, e.g., for the oral delivery of quercetin, that contain at least about 1, 5, 10, 20, 30, 40, 50, 60, 70, 80, 90, 95, 99, 99.5, 99.9, or 99.99% quercetin aglycone. In some embodiments, the invention provides a composition for the oral delivery of quercetin that contains no more than about 2, 5, 10, 20, 30, 40, 50, 60, 70, 80, 90, 95, 99, 99.5, 99.9, 99.99, or 100% quercetin aglycone. In some embodiments, the invention provides a composition that contains about 1-100% quercetin aglycone, or about 10-100% quercetin aglycone, or about 20-100% quercetin aglycone, or about 50-100% quercetin aglycone, or about 80-100% quercetin aglycone, or about 90-100% quercetin aglycone, or about 95-100% quercetin aglycone, or about 99-100% quercetin aglycone. In some embodiments, the invention provides a composition that contains about 1-90% quercetin aglycone, or about 10-90% quercetin aglycone, or about 20-90% quercetin aglycone, or about 50-90% quercetin aglycone, or about 80-90% quercetin aglycone. In some embodiments, the invention provides a composition that contains about 1-75% quercetin aglycone, or about 10-75% quercetin aglycone, or about 20-75% quercetin aglycone, or about 50-75% quercetin aglycone. In some embodiments, the invention provides a composition that contains about 1-50% quercetin aglycone, or about 10-50% quercetin aglycone, or about 20-50% quercetin aglycone, or about 30-50% quercetin aglycone, or about 40-50% quercetin aglycone. In some embodiments, the invention provides a composition that contains about 1-40% quercetin aglycone, or about 10-40% quercetin aglycone, or about 20-40% quercetin aglycone, or about 30-40% quercetin aglycone. In some embodiments, the invention provides a composition that contains about 1-30% quercetin aglycone, or about 10-30% quercetin aglycone, or about 20-30% quercetin aglycone. In some embodiments, the invention provides a composition that contains about 1-20% quercetin aglycone, or about 10-20% quercetin aglycone. In some embodiments, the invention provides a composition that contains about 1-10% quercetin aglycone. In some embodiments, the invention provides a composition that contains about 1, 2, 5, 10, 20, 30, 40, 50, 60, 70, 80, 90, 95, 96, 97, 98, or 99% quercetin aglycone.

In some embodiments, the invention provides a composition for administration of quercetin to an animal to reduce a CNS effect of a substance, e.g., for the oral delivery of quercetin, that contains a combination of quercetin-O-saccharides. In some embodiments, the invention provides a composition for administration of quercetin to an animal to reduce a CNS effect of a substance, e.g., for the oral delivery of quercetin, that contain a combination of quercetin-3-O-glycoside and quercetin-3-O-glucorhamnoside. In these compositions, the ranges or amounts of the quercetin-O-saccharides, e.g., quercetin-3-O-glycoside and quercetin-3-O-glucorhamnoside may be any suitable combination of the ranges or amounts, above.

In some embodiments, the invention provides a composition for administration of quercetin to an animal to reduce a CNS effect of a substance, e.g., for the oral delivery of quercetin, that contains a combination of one or more quercetin-O-saccharides and quercetin aglycone. In some embodiments, the invention provides a composition for administration of quercetin to an animal to reduce a CNS effect of a substance, e.g., for the oral delivery of quercetin, that contain a combination of quercetin-3-O-glycoside and quercetin aglycone. In these compositions, the ranges or amounts of quercetin-3-O-glycoside and quercetin aglycone may be any suitable combination of the ranges or amounts, above. In some embodiments, the invention provides a composition for administration of quercetin to an animal to reduce a CNS effect of a substance, e.g., for the oral delivery of quercetin, that contain a combination of quercetin-3-O-glucorhamnoside and quercetin aglycone. In these compositions, the ranges or amounts of quercetin-3-O-glucorhamnoside and quercetin aglycone may be any suitable combination of the ranges or amounts, above. In some embodiments, the invention provides a composition for administration of quercetin to an animal to reduce a CNS effect of a substance, e.g., for the oral delivery of quercetin, that contain a combination of quercetin-3-O-glycoside, quercetin-3-O-glucorhamnoside and quercetin aglycone. In these compositions, the ranges or amounts of quercetin-3-O-glycoside, quercetin-3-O-glucorhamnoside and quercetin aglycone may be any suitable combination of the ranges or amounts, above. Other quercetin saccharides, as described herein and as known in the art or developed, may be used as well.

In some of these embodiments, a pharmaceutically acceptable excipient is also included.

IV. Substances Whose CNS Effects are Desired to be Reduced (e.g., Drugs)

The invention provides compositions and methods to reduce or eliminate the effects of a substance in the CNS and/or fetus. The substance may be produced in the CNS in a normal or abnormal condition (e.g., beta amyloid in Alzheimer's disease). The substance may be an agent that is introduced into an animal, e.g., a therapeutic agent (e.g., an analgesic for pain relief). It will be appreciated that some therapeutic agents are also agents produced naturally in an animal, and the two groups are not mutually exclusive. In some embodiments, the compositions and methods retain or enhance a desired effect of the substance, e.g., a peripheral effect. The methods and compositions of the invention apply to any therapeutic agent for which it is desired to reduce one or more CNS and/or fetal effects of the agent. In some embodiments, the compositions and methods of the invention utilize an analgesic agent. In some embodiments, the analgesic agent is an opiate analgesic. In some embodiments, the analgesic is a non-opiate analgesic. In some embodiments, the compositions and methods of the invention utilize a non-analgesic therapeutic agent. It will be appreciated that there is some overlap between these groups, as some agents that have primarily an analgesic effect also have other therapeutic effects, while some agents that have primarily a non-analgesic effect also provide some degree of analgesia. The invention encompasses these therapeutic agents as well.

Hence, in some embodiments, the methods and compositions of the present invention can be used to modulate transport of a variety of therapeutic agents. In some embodiments, the dosage of the therapeutic agent will be modulated according to the effect of the transport protein modulator. For instance, less therapeutic agent may be needed to reach optimal effect when co-administered with the transport protein modulator. In another embodiments co-administering the transport protein modulator with a therapeutic agent will allow for chronically administering the drug without drug escalation and/or without dependence on the drug. In another embodiment co-administering the transport protein modulator will allow for the elimination of a therapeutic agent from a physiological compartment, i.e. wash out drug in an overdose situation or to wake up a patient faster after anesthesia. In some embodiments, the physiological compartment is a central nervous system. In some embodiments, the physiological compartment is a fetal compartment.

The term “central nervous system (CNS) effect,” as used herein, encompasses any effect of a substance in the CNS. The effect may be acute or chronic. The effect may be biochemical, cellular, at the tissue level, at the organ level, at the multi-organ level, or at the level of the entire organism. The effect may manifest in one or more objective or subjective manners, any of which may be used to measure the effect. For some substances that may be normally or abnormally produced in the CNS, such as amyloid beta, the effect may be a pathological effect. In some embodiments, the CNS effect of a substance can be drowsiness, impaired concentration, sexual dysfunction, sleep disturbances, habituation, dependence, alteration of mood, respiratory depression, nausea, vomiting, dizziness, memory impairment, neuronal dysfunction, neuronal death, visual disturbances, impaired mentation, tolerance, addiction, hallucinations, lethargy, myoclonic jerking, or endocrinopathies, or combinations thereof.

If an effect is measured objectively or subjectively (e.g., drowsiness, pain, and the like), any suitable method for evaluation of objective or subjective effect may be used. Examples include visual and numeric scales and the like for evaluation by an individual of, e.g., the Likert scale for pain. A further example includes sleep latency for measurement of drowsiness, or standard tests for measurement of concentration, mentation, memory, and the like. These and other methods of objective and subjective evaluation of CNS effects by either an objective observer, the individual, or both, are well-known in the art.

The term “fetal effect,” as used herein, encompasses any effect encompasses any effect of a substance that is introduced into the maternal system on the fetus. The effect may be acute or chronic. The effect may be biochemical, cellular, at the tissue level, at the organ level, at the multi-organ level, or at the level of the entire organism.

A “therapeutic effect,” as that term is used herein, encompasses a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. A prophylactic effect includes delaying or eliminating the appearance of a disease or condition, delaying or eliminating the onset of symptoms of a disease or condition, slowing, halting, or reversing the progression of a disease or condition, or any combination thereof.

The term “physiological compartment” as used herein includes physiological structures, such as organs or organ groups or the fetal compartment, or spaces whereby a physiological or chemical barrier exists to exclude compounds or agents from the internal portion of the physiological structure or space. Such physiological compartments include the central nervous system, the fetal compartment and internal structures contained within organs, such as the ovaries and testes.

A. Analgesic Agents

The compositions and methods of the invention encompass the use of one or more analgesic agents in combination with an agent that reduces a CNS effect of the analgesic, such as a BBB transport protein modulator.

Analgesic agents are agents used to reduce or eliminate pain. An analgesic (colloquially known as painkiller) is any member of the diverse group of drugs used to relieve pain and to achieve analgesia (“absence of pain”). Analgesic drugs act in various ways on the peripheral and central nervous system; analgesics may be employed for symptomatic relief and include broadly two major groups: 1) opiate analgesics; 2) nonopiate analgesics, including analgesics and antipyretics, nonsteroidal antiinflammatory drugs, acetominophen, paracetamol, indomethacin, tricyclic antidepressants (for example desipramine, imipramine, amytriptiline, nortriptile), anticonvulsants (for example, carbamazepine, valproate), and serotonin reuptake inhibitors (for example, fluoxetine, paraoxetine, sertraline), mixed serotonin-norepinephrine reuptake inhibitors (for example venlafaxine, duloxetine), serotonin receptor agonists and antagonists, cholinergic (muscarinic and nicotinic) analgesics, adrenergic agents, and neurokinin antagonists.

In one embodiment analgesic agents are selected from the group consisting of oxycodone, gabapentin, pregabalin, hydrocodone, fentanyl, hydromorphine, levorphenol, morphine, methadone, tramadol and topiramate.

1. Opiate Analgesics

In some embodiments of the invention utilizing an analgesic agent, the analgesic agent is an opiate. Opiates bind stereospecific receptors predominantly in the CNS and peripheral nervous system. The mu, kappa, and delta opiate receptors are the receptors most responsible for the analgesic effects. Mu activation produces analgesia but also has the usually undesired effects of respiratory depression, addiction, and euphoria. Kappa receptors are generally located in the spinal cord and help with spinal analgesia but also cause miosis and sedation. Delta sites are also involved in analgesia. There is no ceiling effect with the analgesia provided by additional amounts of opiates. Thus side-effects also tend to increase with increasing dosage. Most common are gastrointestinal side-effects such as constipation, nausea and gastric distress. Sedation is also common.

Should the pain still prove debilitating, the clinician may choose to use stronger narcotics. Morphine is a pure agonist and makes for an excellent analgesic. Other mixed agonist/antagonist opiates, such as pantazocine, nalbuphine, and butorphanol, will selectively block mu receptors and activate kappa receptors. These drugs do exhibit a ceiling effect. Partial agonists act similarly by activating the mu receptor and block occupation of the kappa site.

Opioid alkaloids used in pain treatment and useful in embodiments of the invention include morphine (morphine sulfate), codeine, and thebaine. Semisynthetic derivatives include diamorphine (heroin), oxycodone, hydrocodone, dihydrocodeine, hydromorphone, oxymorphone, and nicomorphine. Synthetic opioids include phenylheptylamines such as methadone and levomethadyl acetate hydrochloride (LAAM); phenylpiperidines such as pethidine (meperidine), fentanyl, alfentanyl, sufentanil, remifentanil, ketobemidone, and carfentanyl; diphenylpropylamine derivatives such as propoxyphene, dextropropoxyphene, dextromoramide, bezitramide, and piritramide; benzomorphan derivatives such as pentazocine and phenazocine; oripavine derivatives such as buprenorphine; and morphinan derivatives such as butorphanol and nalbufine; and other opioids such as dezocine, etorphine, tilidine, tramadol, loperamide, nalbuphine, dextromethorphan, and diphenoxylate. Analgesic combinations that include opioids include analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin or acetaminophen.

In some embodiments, compositions and methods of the invention encompass the use of an opioid analgesic in combination with an agent that reduces a CNS effect of the opioid analgesic, such as a BBB transport protein modulator. In some embodiments, the opioid is oxycodone, hydrocodone, fentanyl, hydromorphine, levorphenol, morphine, methadone, or tramadol. In some embodiments, the opioid is oxycodone, hydrocodone, methadone, or tramadol. In some embodiments, the opioid is oxycodone. In some embodiments, the opioid is hydrocodone. In some embodiments, the opioid is methadone. In some embodiments, the opioid is tramadol.

True opioids have no ceiling dose, and dosing is often limited by CNS effects. Thus, the compositions and methods of the invention allow greater pain relief by increasing dose, if necessary, without increasing CNS effects or with less increase in CNS effects. In some embodiments, the methods and compositions of the invention allow greater pain relief for a given dose of opioid, in some embodiments together with decreased CNS effects.

2. Non-Opiate Analgesics

In some embodiments, the invention encompasses the use of a non-opiate analgesic. In some embodiments, the non-opiate analgesic is used in combination with an agent that reduces a CNS effect of the non-opiate analgesic. In some embodiments, the non-opiate analgesic is used in addition to another analgesic, in combination with an agent that reduces a CNS effect of the non-opiate analgesic and/or a CNS effect of the other analgesic.

Antidepressants and anticonvulsants In neuropathic and other opioid-insensitive pain conditions, antidepressants, e.g., tricyclic antidepressants (“TCAs”) and anticonvulsant therapy is typically used.

TCAs have been hypothesized to have their own analgesic effect, potentiate narcotics, and treat neuropathic pain as their modes of action for analgesia. Exemplary TCAs include Amitriptyline, Amoxapine, Clomipramine, Desipramine, Doxepin, Imipramine, Nortriptyline, Protriptyline, and Trimipramine.

In addition, other types of antidepressants may be used in treatment of, e.g., chronic pain. These include Escitalopram, Sertraline, Citalopram, Paroxetine, Paroxetin, controlled release, Fluoxetine, Venlafaxine; Reboxetine, Milnacipran, Mirtazapine, Nefazodone, Duloxetin Bupropion, Maprotiline, Mianserin, Trazodone, Dexmethylphenidate, Methyphenidate, and Amineptine, Fluoxetine weekly, Fluvoxamine, olanzapine/fluoxetine combination.

Anticonvulsants such as carbamazapine, topiramate, gabapentin, and pregabalin are used in neuropathic pains such as trigeminal neuralgia. Mexiletine and clonazepam have also been shown to be effective in other neuronally mediated types of pain. Further anticonvulsant agents include clorazepate dipotassium, diazepam, ethosuximide, ethotoin, felbamate, fosphenyloin, lamotrigine, levetiracetam, lorazepam, mephenyloin, mephobarbital, oxycarbazepine, pentobarbital sodium, phenobarbital, phenyloin, primidone, tiagabine, trimethadione, and valproic acid.

In some embodiments, compositions and methods of the invention encompass the use of an anticonvulsant in combination with an agent that reduces a CNS effect of the anticonvulsant, such as a BBB transport protein modulator. In some embodiments, the anticonvulsant is gabapentin, pregabalin, or topiramate. In some embodiments, the anticonvulsant is gabapentin. In some embodiments, the anticonvulsant is pregabalin. In some embodiments, the anticonvulsant is topiramate.

Antiinflammatory compounds, both steroidal and non-steroidal, also find use in pain relief, and may be used in the compositions and methods of the invention.

Non-limiting examples of steroidal anti-inflammatory agents suitable for use herein include corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

Additional nonopiate analgesics of use in the invention include the non-steroidal antiinflammatory compounds. NSAIDS are typically used as analgesics, antipyretics and anti-inflammatories. Acetaminophen, while not normally classified as an NSAID because it is not anti-inflammatory, has similar analgesic effects and is often used similarly. Salicylates are hydrolyzed by the body into salicylic acid whereas salicylamide and diflunisal have structural and functional similarities but do not get hydrolyzed. At sites of inflammation, NSAIDS typically inhibit prostaglandin synthesis by irreversibly acetylating cyclooxygenase and may inhibit nitric oxide synthetase, TNF-alpha, IL-1 and change other lymphocytic activity decreasing inflammation. Diclofenac, ibuprofen, indomethacin, and ketoprofen have been shown to have direct analgesic activity as well. Clinically, NSAIDs are typically used for mild to moderate pain, and are generally considered for some types of pain, most notably post-surgical pain, as being more effective than opioids.

NSAIDS used in pain treatment include salicylates such as aspirin, methyl salicylate, and diflunisal; arylalkanoic acids such as indomethacin, sulindac, diclofenac, and tolmetin; N-arylanthranilic acids (fenamic acids) such as mefenamic acid and mecflofenamate; oxicams such as piroxicam and meloxicam; coxibs such as celecoxib, rofecoxib, valdecoxib, parecoxib, and etoricoxib; sulphonanilides such as nimesulide; naphthylalkanones such as nabumetone; anthranilic acids such as pyrazolidinediones and phenylbutazone; proprionic acids such as fenoprofen, flurbiprofen, ibuprofen, ketoprofen, naproxen, and oxaprozin; pyranocarboxylic acids such as etodolac; pyrrolizine carboxylic acids such as ketorolac; and carboxylic acids.

Sedative-Hypnotic Drugs, may also be used, and include drugs that bind to the GABA_{A} receptor such as the benzodiazepines (including alprazolam, chlordiazepoxide, clorazepate, clonazepam, diazepam, estazolam, flurazepam, halazepam, lorazepam, midazolam, oxazepam, quazepam, temazepam, triazolam), the barbiturates (such as amobarbital, pentobarbital, phenobarbital, secobarbita), and non-benzodiazepines (such as zolpidem and zaleplon), as well as the benzodiazepine antagonists (such as flumazenil). Other sedative-hypnotic drugs appear to work through non-GABA-ergic mechanisms such as through interaction with serotonin and dopaminergic receptors, and include buspirone, isapirone, geprirone, and tandospirone. Older drugs work through mechanisms that are not clearly elucidated, and include chloral hydrate, ethchlorvynol, meprobamate, and paraldehyde.

Ergot alkaloids are useful in the treatment of, e.g., migraine headache, and act on a variety of targets, including alpha adrenoceptors, serotonin receptors, and dopamine receptors. They include bromocriptine, cabergoline, pergolide, ergonovine, ergotamine, lysergic acid diethylamide, and methysergide. Available preparations include dihydroergotamine, ergonovine, ergotamine, ergotamine tartrate, and methylergonovine.

3. Other Pain-Reducing Modalities

In some embodiments, the compositions and methods of the invention encompass the use of an analgesic agent in combination with a modulator of a BBB transport protein, and further in combination with another pain-reducing modality. Treatment may also be by mechanical modalities of massage, ultrasound, stretching, traction, hydrotherapy or application of heat and cold. Electrical modalities of transcutaneous electrical nerve stimulation (TENS) or microcurrent electrical therapy (MET) might be used. Other therapies such as magnetic biostimulation, acupuncture, pulsed signal therapy, physical therapy, and electromedicine have all been used to treat pain conditions. Alternative and Eastern approaches have also been utilized. As part of a pain treatment or diagnosis plan, neural blockade by the introduction of local anesthetic or, rarely, a neurolytic can be used, usually combined with a steroid.

B. Non-Analgesic Agents

The methods and compositions of the invention are also useful in relation to non-analgesic therapeutic agents.

Thus, other suitable drugs for use herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Additional suitable drugs include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease. The compounds and methods of the invention can be used to modulate transport of drugs used to treat protozoal infections, drugs used to treat Malaria, Amebiasis, Giardiasis, Trichomoniasis, Trypanosomiasis, and/or Leishmaniasis, and/or drugs used in the chemotherapy of helminthiasis. Other drugs include antimicrobial agents, sulfonamides, trimethoprim-sulfamethoxazole quinolones, and agents for urinary tract infections, penicillins, cephalosporins, and other, β-Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

In addition, drugs used for immunomodulation, such as immunomodulators, immunosuppressive agents, tolerogens, and immunostimulants can be modulated. In addition, drugs acting on the blood and the blood-forming organs, hematopoietic agents, growth factors, minerals, and vitamins, anticoagulant, thrombolytic, and antiplatelet drugs can also be modulated. The invention can be used to modulate transport of hormones and hormone antagonists, pituitary hormones and their hypothalamic releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, and other compounds. Further transport of vitamins such as water-soluble vitamins, vitamin B complex, ascorbic acid, fat-soluble vitamins, vitamins A, K, and E can be modulated.

Additional suitable drugs may be found in Goodman and Gilman's “The Pharmacological Basis of Therapeutics” Tenth Edition edited by Hardman, Limbird and Gilman or the Physician's Desk Reference, both of which are incorporated herein by reference in their entirety.

Antihypertensives In some embodiments, compositions and methods of the invention encompass the use of an antihypertensive in combination with an agent that reduces a CNS effect of the antihypertensive, such as a BBB transport protein modulator.

Examples of antihypertensives useful in the methods and compositions of the invention include but are not limited to: atenolol, captopril, clonidine, guanethidine, hydralazine, hydrochorothiazide, lisinopril, losartan, methyldopa, minoxidil, nifedipine, prazosin, propranolol, reserpine, verapamil; centrally acting sympathoplegic drugs such as methyldopa, clonidine, guanabenz, guanfacine; ganglion-blocking agents such as mecamylamine (inversine); adrenergic neuron-blocking agents such as guanethidine, guanadrel, bethanidine, debrisoquin, reserpine; adrenoceptor antagonists such as propranolol; other beta-adrenoceptor-blocking agents such as metoprolol, nadolol, carteolol, atenolol, betaxolol, bisoprolol, pindolol, acebutolol, penbutolol, labetalol, carvedilol, esmolol, timolol; prazosin and other alpha blockers such as prazosin, terazosin, doxazosin; other alpha adrenoceptor-blocking agents such as pinacidil, urapidil, cromakalim; nonselective agents, phentolamine and phenoxybenzamine; vasodilators such as hydralazine and minoxidil; sodium nitroprusside, diazoxide, fenoldopam; calcium channel blockers such as verapamil, diltiazem and dihydrophyridine family (amlodipine, felodipine, isradipine, nicardipine, nifedipine, and nisoldipine); inhibitors of angiotensin such as renin, angiotensin, aldosterone; angiotensin-converting enzyme (ACE) inhibitors such as captopril, enalapril, lisinopril, benazepril, fosinopril, moexipril, perindopril, quinapril, ramipril, trandolapril; angiotensin receptor-blocking agents such as losartan, valsartan, candesartan, eprosartan, irbesartan and telmisartan, and olmisartan.

Antiinfectives In some embodiments, compositions and methods of the invention encompass the use of an antiinfective agent in combination with an agent that reduces a CNS effect of the antibacterial agent, such as a BBB transport protein modulator.

Non-limiting examples of antiinfective agents useful in the invention include β-lactam drugs, quinolone drugs, ciprofloxacin, norfloxacin, tetracycline, amikacin, 2,4,4′-trichloro-2′-hydroxy diphenyl ether, 3,4,4′-trichlorocarbanilide, phenoxyethanol, phenoxy propanol, phenoxyisopropanol, doxycycline, capreomycin, chlorhexidine, chlortetracycline, oxytetracycline, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione and clotrimazole

V. Compositions

In one aspect the invention provides compositions that include an agent that reduces or eliminates a central nervous system (CNS) and/or fetal effect of one or more substances. In some embodiments, the substance is a therapeutic agent with which the agent that reduces the CNS effect is co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

In some embodiments, the invention provides compositions containing a combination of a therapeutic agent and an agent that reduces or eliminates a central nervous system (CNS) and/or fetal effect of the therapeutic agent. In some embodiments the invention provides pharmaceutical compositions that further include a pharmaceutically acceptable excipient. In some embodiments, the pharmaceutical compositions are suitable for oral administration. In some embodiments, the pharmaceutical compositions are suitable for transdermal administration. In some embodiments, the pharmaceutical compositions are suitable for injection. Other forms of administration are also compatible with embodiments of the pharmaceutical compositions of the invention, as described herein.

In some embodiments, the BBB transport protein is an ABC transport protein. In some embodiments, the BBB transport protein modulator is an BBB transport protein activator. In some embodiments, the BBB transport protein modulator is a modulator of P-gP.

In some embodiments, the BBB transport protein modulator comprises a polyphenol. In other embodiments, a polyphenol which acts to lower a CNS effect of a therapeutic agent through a non-BBB transport protein-mediated mechanism, or that acts to lower a CNS effect of a therapeutic agent through a BBB transport protein-mediated mechanism and a non-BBB transport protein-mediated mechanism, is used. In some embodiments utilizing a polyphenol, the polyphenol is a flavonoid. In some embodiments utilizing a polyphenol, the polyphenol is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin. In some embodiments utilizing a polyphenol, the polyphenol is a flavonol. In certain embodiments, the flavonol is selected from the group consisting of quercetin, galangin, and kaempferol, or combinations thereof. In some embodiments, the flavonol is quercetin. In some embodiments, the flavonol is galangin. In some embodiments, the flavonol is kaempferol.

In some embodiments, the CNS effect of the therapeutic agent that is reduced is selected from the group consisting of drowsiness, impaired concentration, sexual dysfunction, sleep disturbances, habituation, dependence, alteration of mood, respiratory depression, nausea, vomiting, dizziness, memory impairment, neuronal dysfunction, neuronal death, visual disturbance, impaired mentation, tolerance, addiction, hallucinations, lethargy, myoclonic jerking, endocrinopathies, and combinations thereof. In some embodiments, the CNS effect of the therapeutic agent that is reduced is selected from the group consisting of impaired concentration and sleep disturbances. In some embodiments, the CNS effect of the therapeutic agent that is reduced is impaired concentration. In some embodiments, the CNS effect of the therapeutic agent that is reduced is sleep disturbances.

In some embodiments the therapeutic agent is an analgesic agent. In some embodiments, the analgesic agent is selected from the group consisting of oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, and haloperidol. In some embodiments, the analgesic agent is selected from the group consisting of oxycodone, gabapentin, pregabalin, hydrocodone, fentanyl, hydromorphone, levorphenol, morphine, methadone, topiramate, diacetyl morphine, codeine, olanzapine, hydrocortisone, prednisone, sufentanyl, alfentanyl, carbamazapine, lamotrigine, doxepin, and haloperidol. In some embodiments, the analgesic agent is selected from the group consisting of oxycodone, gabapentin, pregabalin, hydrocodone, fentanyl, hydromorphine, levorphenol, morphine, methadone, tramadol and topiramate. In some embodiments, the analgesic is selected from the group consisting of oxycodone and gabapentin. In some embodiments, the analgesic is oxycodone. In some embodiments, the analgesic is gabapentin. In some embodiments, the analgesic is hydrocodone.

In some embodiments, the analgesic is an opiate analgesic. Opiate analgesics are as described herein. In some embodiments, the composition includes an opiate analgesic selected from the group consisting of oxycodone, hydrocodone, fentanyl, hydromorphone, levorphenol, morphine, methadone, tramadol, diacetyl morphine, codeine, sufentanyl, and alfentanyl. In some embodiments, the composition includes an opiate analgesic selected from the group consisting of oxycodone, hydrocodone, methadone, and tramadol. In some embodiments, the composition includes an opiate analgesic selected from the group consisting of oxycodone, hydrocodone, and methadone. In some embodiments, the opiate analgesic is oxycodone. In some embodiments, the opiate analgesic is hydrocodone. In some embodiments, the opiate analgesic is methadone.

In some embodiments, the analgesic is a non-opiate analgesic. Non-opiate analgesics are as described herein. In some embodiments, the composition includes a non-opiate analgesic selected from the group consisting of gabapentin, pregabalin, topiramate, olanzapine, hydrocortisone, prednisone, carbamazapine, lamotrigine, doxepin, and haloperidol. In some embodiments, the non-opiate analgesic is gabapentin. In some embodiments, the non-opiate analgesic is pregabalin.

Combinations of analgesics, such as combinations of an opiate and non-opiate analgesic, as are known in the art, may also be used in compositions of the invention.

In some embodiments, the composition includes a non-analgesic therapeutic agent. In some embodiments, the non-analgesic therapeutic agent is selected from the group consisting of antihypertensives, vasodilators, barbiturates, membrane stabilizers, cardiac stabilizers, glucocorticoids, antiinfectives. In some embodiments, the non-analgesic therapeutic agent is an antihypertensive. In some embodiments, the non-analgesic therapeutic agent is an antiinfective.

In some embodiments, the invention provides a composition containing a therapeutic agent and an blood-brain barrier (BBB) transport protein modulator, where the therapeutic agent is present in an amount sufficient to exert a therapeutic effect and the BBB transport protein modulator is present in an amount sufficient to decrease a central nervous system (CNS) effect of the therapeutic agent by a measurable amount, compared to the CNS effect without the BBB transport protein modulator, when the composition is administered to an animal. In some embodiments, a CNS effect of the therapeutic agent is decreased by an average of at least about 5, 10, 15, 20, 25, 30, 35, 40, 45, 50, 55, 60, 65, 70, 75, 80, 85, 90, 95, or more than 95%, compared to the CNS effect without the BBB transport protein modulator. In some embodiments, a CNS effect of the therapeutic agent is decreased by an average of at least about 5%, compared to the CNS effect without the BBB transport protein modulator. In some embodiments, a CNS effect of the therapeutic agent is decreased by an average of at least about 10%, compared to the CNS effect without the BBB transport protein modulator. In some embodiments, a CNS effect of the therapeutic agent is decreased by an average of at least about 15%, compared to the CNS effect without the BBB transport protein modulator. In some embodiments, a CNS effect of the therapeutic agent is decreased by an average of at least about 20%, compared to the CNS effect without the BBB transport protein modulator. In some embodiments, a CNS effect is substantially eliminated compared to the CNS effect without the BBB transport protein modulator. “Substantially eliminated” as used herein encompasses no measurable or no statistically significant CNS effect (one or more CNS effects) of the therapeutic agent, when administered in combination with the BBB transport protein modulator.

Thus, in some embodiments, the invention provides compositions that contain a polyphenol, e.g., a flavonol, and an analgesic agent, where the analgesic agent is present in an amount sufficient to exert an analgesic effect and the polyphenol, e.g., a flavonol is present in an amount sufficient to decrease a central nervous system (CNS) effect of the analgesic agent by a measurable amount, compared to the CNS effect without the polyphenol, e.g., a flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is disturbance of concentration. In some embodiments, the CNS effect is sleep disturbances.

In some embodiments, the invention provides compositions that contain a flavonol and an opiate analgesic agent, where the opiate analgesic agent is present in an amount sufficient to exert an analgesic effect and the flavonol is present in an amount sufficient to decrease a central nervous system (CNS) effect of the opiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and an opiate analgesic agent that is oxycodone, hydrocodone, fentanyl, hydromorphone, levorphenol, morphine, methadone, tramadol, diacetyl morphine, codeine, sufentanyl, and alfentanyl, or a combination thereof, where the opiate analgesic agent is present in an amount sufficient to exert an analgesic effect and the flavonol is present in an amount sufficient to decrease a central nervous system (CNS) effect of the opiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contain a flavonol that is quercetin, galangin, or kaempferol, or combination thereof, and an opiate analgesic agent that is oxycodone, methadone, hydrocodone, or tramadol, or a combination thereof, where the opiate analgesic agent is present in an amount sufficient to exert an analgesic effect and the flavonol is present in an amount sufficient to decrease a central nervous system (CNS) effect of the opiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contains quercetin and oxycodone where the oxycodone is present in an amount sufficient to exert an analgesic effect and the quercetin is present in an amount sufficient to decrease a central nervous system (CNS) effect of the oxycodone by a measurable amount, compared to the CNS effect without the quercetin when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contain a flavonol and a nonopiate analgesic agent, where the nonopiate analgesic agent is present in an amount sufficient to exert an analgesic effect and the flavonol is present in an amount sufficient to decrease a central nervous system (CNS) effect of the nonopiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contain a flavonol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or a combination thereof, and an nonopiate analgesic agent that is gabapentin, pregabalin, topiramate, olanzapine, hydrocortisone, prednisone, carbamazapine, lamotrigine, doxepin, or haloperidol, or a combination thereof, where the nonopiate analgesic agent is present in an amount sufficient to exert an analgesic effect and the flavonol is present in an amount sufficient to decrease a central nervous system (CNS) effect of the nonopiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contain a flavonol that is quercetin, galangin, or kaempferol, or combination thereof, and an nonopiate analgesic agent that is gabapentin, lorazepam, cyclobenzaprine hydrochloride, or carisoprodol, where the nonopiate analgesic agent is present in an amount sufficient to exert an analgesic effect and the flavonol is present in an amount sufficient to decrease a central nervous system (CNS) effect of the nonopiate analgesic agent by a measurable amount, compared to the CNS effect without the flavonol when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contains quercetin and gabapentin where the gabapentin is present in an amount sufficient to exert an analgesic effect and the quercetin is present in an amount sufficient to decrease a central nervous system (CNS) effect of the gabapentin by a measurable amount, compared to the CNS effect without the quercetin when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the invention provides compositions that contains quercetin and pregabalin where the pregabalin is present in an amount sufficient to exert an analgesic effect and the quercetin is present in an amount sufficient to decrease a central nervous system (CNS) effect of the pregabalin by a measurable amount, compared to the CNS effect without the quercetin when the composition is administered to an animal. The measurable amount may be an average of at least about 5%, 10%, 15%, 20%, or more than 20% as described herein. The CNS effect may be any CNS effect as described herein. In some embodiments, the CNS effect is loss of concentration. In some embodiments, the CNS effect is sleep disturbances.

In some embodiments, the BBB transport protein modulator is present in an amount sufficient to decrease a central nervous system (CNS) effect of the therapeutic agent by a measurable amount and to increase a therapeutic effect of the therapeutic agent by a measurable amount, compared to the CNS effect and therapeutic effect without the BBB transport protein modulator, when the composition is administered to an animal. In some embodiments, a therapeutic effect of the therapeutic agent is increased by an average of at least about 5, 10, 15, 20, 25, 30, 35, 40, 45, 50, 55, 60, 65, 70, 75, 80, 85, 90, 95, or more than 95%, compared to the therapeutic effect without the BBB transport protein modulator. In some embodiments, a therapeutic effect of the therapeutic agent is increased by an average of at least about 5%, compared to the therapeutic effect without the BBB transport protein modulator. In some embodiments, a therapeutic effect of the therapeutic agent is increased by an average of at least about 10%, compared to the therapeutic effect without the BBB transport protein modulator. In some embodiments, a therapeutic effect of the therapeutic agent is increased by an average of at least about 15%, compared to the therapeutic effect without the BBB transport protein modulator. In some embodiments, a therapeutic effect of the therapeutic agent is increased by an average of at least about 20%, compared to the therapeutic effect without the BBB transport protein modulator. In some embodiments, a therapeutic effect of the therapeutic agent is increased by an average of at least about 30%, compared to the therapeutic effect without the BBB transport protein modulator. In some embodiments, a therapeutic effect of the therapeutic agent is increased by an average of at least about 40%, compared to the therapeutic effect without the BBB transport protein modulator. In some embodiments, a therapeutic effect of the therapeutic agent is increased by an average of at least about 50%, compared to the therapeutic effect without the BBB transport protein modulator.

Thus, in some embodiments, the invention provides compositions containing a BBB transport protein modulator present in an amount sufficient to decrease a central nervous system (CNS) effect of a therapeutic agent by an average of at least about 5% and to increase a therapeutic effect of the therapeutic agent by an average of at least about 5%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 10% and to increase a therapeutic effect of the therapeutic agent by an average of at least about 10%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 20% and to increase a therapeutic effect of the therapeutic agent by an average of at least about 20%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 10% and to increase a therapeutic effect of the therapeutic agent by an average of at least about 20%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 10% and to increase a therapeutic effect of the therapeutic agent by an average of at least about 30%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 10% and to increase a therapeutic effect of the therapeutic agent by an average of at least about 40%, compared to the CNS effect and therapeutic effect without the BBB transport protein modulator, when the composition is administered to an animal in combination with the therapeutic agent. In some embodiments, the invention provides compositions containing a BBB transport protein modulator present in an amount sufficient to decrease a central nervous system (CNS) effect of a therapeutic agent by an average of at least about 10% and to increase a therapeutic effect of the therapeutic agent by an average of at least about 50%, compared to the CNS effect and therapeutic effect without the BBB transport protein modulator, when the composition is administered to an animal in combination with the therapeutic agent.

In some embodiments, the invention provides compositions containing a polyphenol, e.g., a flavonol such as quercetin, present in an amount sufficient to decrease a central nervous system (CNS) effect of a therapeutic agent by an average of at least about 5% and to increase a therapeutic effect of the therapeutic agent by an average of at least about 5%, when the composition is administered to an animal in combination with the therapeutic agent, compared to the CNS effect and therapeutic effect without the polyphenol, e.g., flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 10% and to increase a therapeutic effect of the therapeutic agent by an average of at least about 10%, when the composition is administered to an animal in combination with the therapeutic agent, compared to the CNS effect and therapeutic effect when the therapeutic agent is administered without the a polyphenol, e.g., a flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 20% and to increase a therapeutic effect of the therapeutic agent by an average of at least about 20%, when the composition is administered to an animal in combination with the therapeutic agent, compared to the CNS effect and therapeutic effect when the therapeutic agent is administered without the a polyphenol, e.g., a flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 10% and to increase a therapeutic effect of the therapeutic agent by an average of at least about 20%, when the composition is administered to an animal in combination with the therapeutic agent, compared to the CNS effect and therapeutic effect when the therapeutic agent is administered without the a polyphenol, e.g., a flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 10% and to increase a therapeutic effect of the therapeutic agent by an average of at least about 30%, when the composition is administered to an animal in combination with the therapeutic agent, compared to the CNS effect and therapeutic effect when the therapeutic agent is administered without the polyphenol, e.g., a flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 10% and to increase a therapeutic effect of the therapeutic agent by an average of at least about 40%, when the composition is administered to an animal in combination with the therapeutic agent, compared to the CNS effect and therapeutic effect when the therapeutic agent is administered without the polyphenol, e.g., a flavonol such as quercetin. In some embodiments, the invention provides compositions containing a polyphenol, e.g., a flavonol such as quercetin present in an amount sufficient to decrease a central nervous system (CNS) effect of a therapeutic agent by an average of at least about 10% and to increase a therapeutic effect of the therapeutic agent by an average of at least about 50%, when the composition is administered to an animal in combination with the therapeutic agent, compared to the CNS effect and therapeutic effect when the therapeutic agent is administered without the a polyphenol, e.g., a flavonol such as quercetin.

In exemplary embodiments, the invention provides a composition that contains a polyphenol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin, or combinations thereof, and an analgesic, such as an opiate or nonopiate analgesic agent, where the analgesic is present in an amount sufficient to exert an analgesic effect, and the polyphenol is present in an amount effective to decrease a CNS effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In exemplary embodiments, the invention provides a composition that contains a flavonol that is quercetin, galangin, or kaempferol and an analgesic that is oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol, where the analgesic is present in an amount sufficient to exert an analgesic effect, and the polyphenol is present in an amount effective to decrease a CNS effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In exemplary embodiments, the invention provides a composition that contains a flavonol that is quercetin, galangin, or kaempferol and an analgesic that is oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol, where the analgesic is present in an amount sufficient to exert an analgesic effect, and the flavonol is present in an amount effective to decrease a CNS effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In further exemplary embodiments, the invention provides a composition that contains a flavonol that is quercetin, galangin, or kaempferol and an analgesic that is oxycodone, hydrocodone, methadone, tramadol, gabapentin, lorazepam, cyclobenzaprine hydrochloride, or carisoprodol, where the analgesic is present in an amount sufficient to exert an analgesic effect, and the flavonol is present in an amount effective to decrease a CNS effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In yet further exemplary embodiments, the invention provides a composition that contains a flavonol that is quercetin, galangin, or kaempferol and an analgesic that is oxycodone or gabapentin, where the analgesic is present in an amount sufficient to exert an analgesic effect, and the flavonol is present in an amount effective to decrease a CNS effect of the analgesic agent by a measurable amount (e.g., an average of at least about 5, 10, 15, 20, or more than 20%, as described herein) and to increase the analgesic effect of the analgesic agent by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In still yet further exemplary embodiments, the invention provides a composition that contains quercetin and oxycodone, where the oxycodone is present in an amount sufficient to exert an analgesic effect, and the quercetin is present in an amount effective to decrease a CNS effect of the oxycodone by a measurable amount (e.g., an average of at least about 5, 10, 15, 20, or more than 20%, as described herein) and to increase the analgesic effect of the oxycodone by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In still yet further exemplary embodiments, the invention provides a composition that contains quercetin and gabapentin, where the gabapentin is present in an amount sufficient to exert an analgesic effect, and the quercetin is present in an amount effective to decrease a CNS effect of the gabapentin by a measurable amount (e.g., an average of at least about 5, 10, 15, 20, or more than 20%, as described herein) and to increase the analgesic effect of the gabapentin by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

In still yet further exemplary embodiments, the invention provides a composition that contains quercetin and pregabalin, where the pregabalin is present in an amount sufficient to exert an analgesic effect, and the quercetin is present in an amount effective to decrease a CNS effect of the pregabalin by a measurable amount (e.g., an average of at least about 5, 10, 15, 20, or more than 20%, as described herein) and to increase the analgesic effect of the pregabalin by a measurable amount (e.g., an average of at least about 5, 10, 15, 20, or more than 20%, as described herein). The CNS effect may be any CNS effect as described herein. In some embodiments, the CNS effect is loss of concentration. In some embodiments, the CNS effect is sleep disturbances.

An “average” as used herein is preferably calculated in a set of normal human subjects, this set being at least about 3 human subjects, preferably at least about 5 human subjects, preferably at least about 10 human subjects, even more preferably at least about 25 human subjects, and most preferably at least about 50 human subjects.

In some embodiments, the invention provides a composition that contains a therapeutic agent and a BBB transport protein modulator, e.g. a polyphenol such as a flavonoid. In some embodiments, the a concentration of one or more of the therapeutic agents and/or BBB transport protein modulator, e.g. a polyphenol such as a flavonol is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0,002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, a concentration of one or more of the therapeutic agents and/or BBB transport protein modulator, e.g. a polyphenol such as a flavonoid is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, a concentration of one or more of the therapeutic agents and/or BBB transport protein modulator, e.g. a polyphenol such as a flavonoid is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v, v/v.

In some embodiments, a concentration of one or more of the therapeutic agents and/or BBB transport protein modulator, e.g. a polyphenol such as a flavonoid is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, a concentration of one or more of the therapeutic agents and/or BBB transport protein modulator, e.g. a polyphenol such as a flavonoid is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, a concentration of one or more of the therapeutic agents and/or BBB transport protein modulator, e.g. a polyphenol such as a flavonoid is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, a concentration of one or more of the therapeutic agents and/or BBB transport protein modulator, e.g. a polyphenol such as a flavonoid is in the range of 0.0001-10 g, 0.0005-9 g, 0.001-8 g, 0.005-7 g, 0.01-6 g, 0.05-5 g, 0.1-4 g, 0.5-4 g, or 1-3 g.

In exemplary embodiments, compositions of the invention include quercetin and oxycodone, where the quercetin is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and the oxycodone is present in an amount from 1 to 200 mg, or about 5-160 mg, or about 2.5, 5, 10, 15, 20, 30, 40, 80, or 160 mg. In some embodiments, the oxycodone/quercetin is present at about 1/50 mg (oxycodone/quercetin). In some embodiments, the oxycodone is present at about 5 mg and the quercetin is present at about 100 mg. In some embodiments, the oxycodone is present at about 5 mg and the quercetin is present at about 250 mg. In some embodiments, the oxycodone is present at about 5 mg and the quercetin is present at about 500 mg. In some embodiments, the oxycodone is present at about 5 mg and the quercetin is present at about 1000 mg. In some embodiments, the oxycodone is present at about 15 mg and the quercetin is present at about 100 mg. In some embodiments, the oxycodone is present at about 15 mg and the quercetin is present at about 250 mg. In some embodiments, the oxycodone is present at about 15 mg and the quercetin is present at about 500 mg. In some embodiments, the oxycodone is present at about 15 mg and the quercetin is present at about 1000 mg. In some embodiments, the oxycodone is present at about 30 mg and the quercetin is present at about 100 mg. In some embodiments, the oxycodone is present at about 30 mg and the quercetin is present at about 200 mg. In some embodiments, the oxycodone is present at about 30 mg and the quercetin is present at about 300 mg. In some embodiments, the oxycodone is present at about 30 mg and the quercetin is present at about 1000 mg.

In, e.g., sustained release preparations, oxycodone (e.g., OXYCONTIN) is present at about 5-200 mg, or at about 10-160 mg, or at about 10, 20, 40, 80 or 160 mg, and quercetin is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg. In some embodiments, oxycodone is present at about 10 mg, and quercetin is present at about 100 mg. In some embodiments, oxycodone is present at about 10 mg, and quercetin is present at about 500 mg. In some embodiments, oxycodone is present at about 10 mg, and quercetin is present at about 1000 mg. In some embodiments, oxycodone is present at about 20 mg, and quercetin is present at about 100 mg. In some embodiments, oxycodone is present at about 20 mg, and quercetin is present at about 500 mg. In some embodiments, oxycodone is present at about 20 mg, and quercetin is present at about 1000 mg. In some embodiments, oxycodone is present at about 40 mg, and quercetin is present at about 100 mg. In some embodiments, oxycodone is present at about 40 mg, and quercetin is present at about 500 mg. In some embodiments, oxycodone is present at about 40 mg, and quercetin is present at about 1000 mg. In some embodiments, oxycodone is present at about 80 mg, and quercetin is present at about 100 mg. In some embodiments, oxycodone is present at about 80 mg, and quercetin is present at about 500 mg. In some embodiments, oxycodone is present at about 80 mg, and quercetin is present at about 1000 mg. In some embodiments, oxycodone is present at about 160 mg, and quercetin is present at about 100 mg. In some embodiments, oxycodone is present at about 160 mg, and quercetin is present at about 500 mg. In some embodiments, oxycodone is present at about 160 mg, and quercetin is present at about 1000 mg.

In liquid preparations, the oxycodone can be present at about 1-100 mg/ml, or 1-50 mg/ml, or 1-20 mg/ml, or about 1, 5, 10, or 20 mg/ml and quercetin at about 1-1000 mg/ml, or about 10-1000 mg/ml, or about 50-1000 mg/ml, or about 100-1000 mg/ml, or about 1-500 mg/ml, or about 5-500 mg/ml, or about 50-500 mg/ml, or about 100-500 mg/ml, or about 200-1000 mg/ml, or about 200-800 mg/ml, or about 200-700 mg/ml, or about 10 mg/ml, or about 25 mg/ml, or about 50 mg/ml, or about 100 mg/ml, or about 200 mg/ml, or about 250 mg/ml, or about 300 mg/ml, or about 400 mg/ml, or about 500 mg/ml, or about 600 mg/ml, or about 700 mg/ml, or about 800 mg/ml, or about 900 mg/ml, or about 1000 mg/ml At higher levels of quercetin, solubility can be enhanced by adjusting the type of diluent.

Oxycodone/quercetin compositions can further include another analgesic, e.g., acetaminophen. Typical dose ratios in such compositions are known in the art, e.g., oxycodone/acetaminophen of about 2.5/325 mg, 5/325 mg, or 5/500 mg, or 7.5/325 mg, or 7.5/500 mg, or 10/325 mg, or 10/650 mg. Any of these compositions may further include quercetin at a dose of about 10 to 1000 mg, or about 50 to 500 mg, or about 50-200 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg

In exemplary embodiments, compositions of the invention include quercetin and gabapentin, where the quercetin is present in an amount from about 1-1000 mg, or about 10-1000 mg, or about 50-1000 mg, or about 100-1000 mg, or about 1-500 mg, or about 5-500 mg, or about 50-500 mg, or about 100-500 mg, or about 200-1000 mg, or about 200-800 mg, or about 200-700 mg, or about 10 mg, or about 25 mg, or about 50 mg, or about 100 mg, or about 200 mg, or about 250 mg, or about 300 mg, or about 400 mg, or about 500 mg, or about 600 mg, or about 700 mg, or about 800 mg, or about 900 mg, or about 1000 mg, and the gabapentin is present in an amount from about 100-2000 mg, or about 100-800 mg, or about 100, 300, 400, 600, or 800 mg. In some embodiments, the gabapentin is present at about 100 mg and the quercetin is present at about 100 mg. In some embodiments, the gabapentin is present at about 100 mg and the quercetin is present at about 200 mg. In some embodiments, the gabapentin is present at about 100 mg and the quercetin is present at about 300 mg. In some embodiments, the gabapentin is present at about 100 mg and the quercetin is present at about 400 mg. In some embodiments, the gabapentin is present at about 100 mg and the quercetin is present at about 500 mg. In some embodiments, the gabapentin is present at about 100 mg and the quercetin is present at about 600 mg. In some embodiments, the gabapentin is present at about 100 mg and the quercetin is present at about 700 mg. In some embodiments, the gabapentin is present at about 100 mg and the quercetin is present at about 800 mg. In some embodiments, the gabapentin is present at about 100 mg and the quercetin is present at about 900 mg. In some embodiments, the gabapentin is present at about 100 mg and the quercetin is present at about 1000 mg. In some embodiments, the gabapentin is present at about 300 mg and the quercetin is present at about 100 mg. In some embodiments, the gabapentin is present at about 300 mg and the quercetin is present at about 200 mg. In some embodiments, the gabapentin is present at about 300 mg and the quercetin is present at about 300 mg. In some embodiments, the gabapentin is present at about 300 mg and the quercetin is present at about 400 mg. In some embodiments, the gabapentin is present at about 300 mg and the quercetin is present at about 500 mg. In some embodiments, the gabapentin is present at about 300 mg and the quercetin is present at about 600 mg. In some embodiments, the gabapentin is present at about 300 mg and the quercetin is present at about 700 mg. In some embodiments, the gabapentin is present at about 300 mg and the quercetin is present at about 800 mg. In some embodiments, the gabapentin is present at about 300 mg and the quercetin is present at about 900 mg. In some embodiments, the gabapentin is present at about 300 mg and the quercetin is present at about 1000 mg. In some embodiments, the gabapentin is present at about 400 mg and the quercetin is present at about 100 mg. In some embodiments, the gabapentin is present at about 400 mg and the quercetin is present at about 200 mg. In some embodiments, the gabapentin is present at about 400 mg and the quercetin is present at about 300 mg. In some embodiments, the gabapentin is present at about 400 mg and the quercetin is present at about 400 mg. In some embodiments, the gabapentin is present at about 400 mg and the quercetin is present at about 500 mg. In some embodiments, the gabapentin is present at about 400 mg and the quercetin is present at about 600 mg. In some embodiments, the gabapentin is present at about 400 mg and the quercetin is present at about 700 mg. In some embodiments, the gabapentin is present at about 400 mg and the quercetin is present at about 800 mg. In some embodiments, the gabapentin is present at about 400 mg and the quercetin is present at about 900 mg. In some embodiments, the gabapentin is present at about 400 mg and the quercetin is present at about 1000 mg. In some embodiments, the gabapentin is present at about 600 mg and the quercetin is present at about 100 mg. In some embodiments, the gabapentin is present at about 600 mg and the quercetin is present at about 200 mg. In some embodiments, the gabapentin is present at about 600 mg and the quercetin is present at about 300 mg. In some embodiments, the gabapentin is present at about 600 mg and the quercetin is present at about 400 mg. In some embodiments, the gabapentin is present at about 600 mg and the quercetin is present at about 500 mg. In some embodiments, the gabapentin is present at about 600 mg and the quercetin is present at about 600 mg. In some embodiments, the gabapentin is present at about 600 mg and the quercetin is present at about 700 mg. In some embodiments, the gabapentin is present at about 600 mg and the quercetin is present at about 800 mg. In some embodiments, the gabapentin is present at about 600 mg and the quercetin is present at about 900 mg. In some embodiments, the gabapentin is present at about 600 mg and the quercetin is present at about 1000 mg. In some embodiments, the gabapentin is present at about 800 mg and the quercetin is present at about 100 mg. In some embodiments, the gabapentin is present at about 800 mg and the quercetin is present at about 200 mg. In some embodiments, the gabapentin is present at about 800 mg and the quercetin is present at about 300 mg. In some embodiments, the gabapentin is present at about 800 mg and the quercetin is present at about 400 mg. In some embodiments, the gabapentin is present at about 800 mg and the quercetin is present at about 500 mg. In some embodiments, the gabapentin is present at about 800 mg and the quercetin is present at about 600 mg. In some embodiments, the gabapentin is present at about 800 mg and the quercetin is present at about 700 mg. In some embodiments, the gabapentin is present at about 800 mg and the quercetin is present at about 800 mg. In some embodiments, the gabapentin is present at about 800 mg and the quercetin is present at about 900 mg. In some embodiments, the gabapentin is present at about 800 mg and the quercetin is present at about 1000 mg.

In liquid preparations, the gabapentin can be present at about 5-500 mg/ml, or about 100-500 mg/ml, or about 250 mg/ml, and quercetin at about 1-1000 mg/ml, or about 10-1000 mg/ml, or about 50-1000 mg/ml, or about 100-1000 mg/ml, or about 1-500 mg/ml, or about 5-500 mg/ml, or about 50-500 mg/ml, or about 100-500 mg/ml, or about 200-1000 mg/ml, or about 200-800 mg/ml, or about 200-700 mg/ml, or about 10 mg/ml, or about 25 mg/ml, or about 50 mg/ml, or about 100 mg/ml, or about 200 mg/ml, or about 250 mg/ml, or about 300 mg/ml, or about 400 mg/ml, or about 500 mg/ml, or about 600 mg/ml, or about 700 mg/ml, or about 800 mg/ml, or about 900 mg/ml, or about 1000 mg/ml At higher levels of quercetin, solubility can be enhanced by adjusting the type of diluent.

In some embodiments, a molar ratio of one or more of the therapeutic agents to the BBB transport protein modulator, e.g. a polyphenol such as a flavonoid can be 0.0001:1 to 1:1. Without limiting the scope of the invention, the molar ratio of one or more of the therapeutic agents to the BBB transport protein modulator, e.g. a polyphenol such as a flavonoid can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1.

Without limiting the scope of the present invention, the molar ratio of one or more of the therapeutic agents to the flavonoid can be about 0.03×10−5:1, 0.1×10−5:1, 0.04×10−3:1, 0.03×10−5:1, 0.02×10−5:1, 0.01×10−3:1, 0.1×10−3:1, 0.15×10−3:1, 0.2×10−3:1, 0.3×10−3:1, 0.4×10−3:1, 0.5×10−3:1, 0.15×10−2:1, 0.1×10−2:1, 0.2×10−2:1, 0.3×10−2:1, 0.4×10−2:1, 0.5×10−2:1, 0.6×10−2:1, 0.8×10−2:1, 0.01:1, 0.1:1; or 0.2:1 per dose. In one embodiment, the therapeutic agent is oxycodone. In one embodiment, the flavonoid is quercetin.

Without limiting the scope of the present invention, the molar ratio of one or more of the therapeutic agents to the flavonoid can be about 0.03×10−5:1, 0.1×10−5:1, 0.04×10−3:1, 0.03×10−5:1, 0.02×10−5:1, 0.01×10−3:1, 0.1×10−3:1, 0.15×10−3:1, 0.2×10−3:1, 0.3×10−3:1, 0.4×10−3:1, 0.5×10−3:1, 0.15×10−2:1, 0.1×10−2:1, 0.2×10−2:1, 0.3×10−2:1, 0.4×10−2:1, 0.5×10−2:1, 0.6×10−2:1, 0.8×10−2:1, 0.01:1, 0.1:1; or 0.2:1 per dose. In one embodiment, the therapeutic agent is fentanyl. In one embodiment, the flavonoid is quercetin.

Without limiting the scope of the present invention, the molar ratio of one or more of the therapeutic agents to the BBB transport protein modulator, e.g. a polyphenol such as a flavonoid can be about 0.001:1, 0.002:1, 0.003:1, 0.004:1, 0.005:1, 0.006:1, 0.007:1, 0.008:1, 0.009:1, 0.01:1, 0.02:1, 0.03:1, 0.04:1, 0.05:1, 0.06:1, 0.07:1, 0.08:1, 0.09:1, 0.1:1, 0.2:1, 0.3:1, 0.4:1, 0.5:1, 0.6:1, 0.7:1, 0.8:1, 0.9:1, 1:1, 2:1, 3:1, 4:1, or 5:1 per dose. In one embodiment, the therapeutic agent is Gabapentin or pregabalin. In one embodiment, the flavonoid is quercetin.

A. Pharmaceutical Compositions

The transport protein modulators of the invention are usually administered in the form of pharmaceutical compositions. The drugs described above are also administered in the form of pharmaceutical compositions. When the transport protein modulators and the drugs are used in combination, both components may be mixed into a preparation or both components may be formulated into separate preparations to use them in combination separately or at the same time.

This invention therefore provides pharmaceutical compositions that contain, as the active ingredient, a BBB transport protein modulator or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

This invention further provides pharmaceutical compositions that contain, as the active ingredient, a BBB transport protein modulator or a pharmaceutically acceptable salt and/or coordination complex thereof, a therapeutic agent or a pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

Such compositions are prepared in a manner well known in the pharmaceutical art.

Pharmaceutical compositions for oral administration In some embodiments, the invention provides a pharmaceutical composition for oral administration containing a combination of a therapeutic agent and an agent that reduces or eliminates a central nervous system (CNS) and/or fetal effect of the therapeutic agent, and a pharmaceutical excipient suitable for oral administration. In some embodiments, the agent that reduces or eliminates the CNS and/or fetal effect of the therapeutic agent is a BBB transport protein modulator, e.g. a polyphenol such as a flavonol, as described elsewhere herein.

In some embodiments, the invention provides a solid pharmaceutical composition for oral administration containing:
(i) an effective amount of a therapeutic agent;
(ii) an effective amount of an agent capable of reducing or eliminating one or more CNS effects of the therapeutic agent; and
(iii) a pharmaceutical excipient suitable for oral administration.

In some embodiments, the composition further contains: (iv) an effective amount of a second therapeutic agent.

In some embodiments, the pharmaceutical composition may be a liquid pharmaceutical composition suitable for oral consumption.

In some embodiments, the therapeutic agent is an analgesic agent. In some embodiments, the therapeutic agent is a non-analgesic agent. In some embodiments, the therapeutic agent is an opiate analgesic agent. In some embodiments, the therapeutic agent is an nonopiate analgesic agent. In some embodiments, the agent capable of reducing or eliminating one or more CNS effects of the therapeutic agent is a BBB transport protein modulator, e.g., a BBB transport protein activator. In some embodiments, the agent capable of reducing or eliminating one or more CNS effects of the therapeutic agent is a polyphenol, e.g., a flavonoid such as a flavonol.

In some embodiments, the invention provides a solid pharmaceutical composition for oral administration containing:
(i) an effective amount of a therapeutic agent that is oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, or haloperidol;
(ii) an effective amount of a polyphenol that is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin; and
(iii) a pharmaceutical excipient suitable for oral administration.

In some embodiments, the composition further contains (iv) an effective amount of a second therapeutic agent. Exemplary second therapeutic agents include aspirin, acetaminophen, and ibuprofen.

In some embodiments, the pharmaceutical composition may be a liquid pharmaceutical composition suitable for oral consumption.

In some embodiments, the invention provides a solid pharmaceutical composition for oral administration containing:
(i) an effective amount of a therapeutic agent that is oxycodone, gabapentin, pregabalin, hydrocodone, fentanyl, hydromorphine, levorphenol, morphine, methadone, tramadol or topiramate;
(ii) an effective amount of a polyphenol that is quercetin, galangin, or kaempferol; and
(iii) a pharmaceutical excipient suitable for oral administration.

In some embodiments, the composition further contains (iv) an effective amount of a second therapeutic agent. Exemplary second therapeutic agents include aspirin, acetaminophen, and ibuprofen.

In some embodiments, the pharmaceutical composition may be a liquid pharmaceutical composition suitable for oral consumption.

In some embodiments, the invention provides a solid pharmaceutical composition for oral administration containing an effective amount of oxycodone, an amount of quercetin that is effective in reducing or eliminating a CNS effect of oxycodone, and a pharmaceutically acceptable excipient. In some embodiments, the composition further includes an effective amount of acetaminophen. In some embodiments, the invention provides a liquid pharmaceutical composition for oral administration containing an effective amount of oxycodone, an amount of quercetin that is effective in reducing or eliminating a CNS effect of oxycodone, and a pharmaceutically acceptable excipient. In some embodiments, the composition further includes an effective amount of acetaminophen.

In some embodiments, the invention provides a solid pharmaceutical composition for oral administration containing oxycodone at about 1-160 mg, quercetin at about 10-1000 mg and a pharmaceutically acceptable excipient. In some embodiments, the composition further includes acetaminophen at about 200-750 mg. In some embodiments, the invention provides a liquid pharmaceutical composition for oral administration containing oxycodone at about 1-200 mg/ml, quercetin at about 10-1000 mg/ml and a pharmaceutically acceptable excipient. In some embodiments, the composition further includes acetaminophen at about 10-750 mg/ml.

In some embodiments, the invention provides a solid pharmaceutical composition for oral administration containing an effective amount of gabapentin, an amount of quercetin that is effective in reducing or eliminating a CNS effect of gabapentin, and a pharmaceutically acceptable excipient. In some embodiments, the invention provides a liquid pharmaceutical composition for oral administration containing an effective amount of gabapentin, an amount of quercetin that is effective in reducing or eliminating a CNS effect of gabapentin, and a pharmaceutically acceptable excipient.

In some embodiments, the invention provides a solid pharmaceutical composition for oral administration containing gabapentin at about 100-800 mg, quercetin at about 10-1000 mg and a pharmaceutically acceptable excipient. In some embodiments, the invention provides a liquid pharmaceutical composition for oral administration containing gabapentin at about 5-500 mg/ml, quercetin at about 10-1000 mg/ml and a pharmaceutically acceptable excipient.

Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphafidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof, polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-100 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the therapeutic agent and/or BBB transport protein modulator (e.g., flavonol) and to minimize precipitation of the therapeutic agent and/or BBB transport protein modulator (e.g., flavonol). This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Pharmaceutical compositions for injection In some embodiments, the invention provides a pharmaceutical composition for injection containing a combination of a therapeutic agent and an agent that reduces or eliminates a central nervous system (CNS) and/or fetal effect of the therapeutic agent, and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the transport protein modulator and/or the therapeutic agent in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical compositions for topical (e.g., transdermal) delivery In some embodiments, the invention provides a pharmaceutical composition for transdermal delivery containing a combination of a therapeutic agent and an agent that reduces or eliminates a central nervous system (CNS) and/or fetal effect of the therapeutic agent, and a pharmaceutical excipient suitable for transdermal delivery. In some embodiments, the agent that reduces or eliminates the CNS and/or fetal effect of the therapeutic agent is a BBB transport protein modulator, e.g. a polyphenol such as a flavonol, as described elsewhere herein. Components and amounts of agents in the compositions are as described herein.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another preferred formulation for use in the methods of the present invention employs transdermal delivery devices (“patches”). Such transdermal patches may be used to provide continuous or discontinuous infusion of the transport protein modulator in controlled amounts, either with or without therapeutic agent. Thus, in some embodiments the invention provides a transdermal patch incorporating a BBB transport protein modulator, e.g., a polyphenol such as a flavonoid (e.g., quercetin). In some embodiments the invention provides a transdermal patch incorporating a BBB transport protein modulator, e.g., a polyphenol such as a flavonoid (e.g., quercetin) in combination with a therapeutic agent, e.g. an analgesic such as an opioid analgesic.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

Pharmaceutical compositions for inhalation. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

Other pharmaceutical compositions Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

B. Kits

The invention also provides kits. The kits include an agent that reduces or eliminates a CNS effect and/or fetal effect of a therapeutic agent, in suitable packaging, and written material that can include instructions for use, discussion of clinical studies, listing of side effects, and the like. The kit may further contain a therapeutic agent that has a CNS effect. In some embodiments, the therapeutic agent and the agent that reduces or eliminates a CNS effect of the therapeutic agent are provided as separate compositions in separate containers within the kit. In some embodiments, the therapeutic agent and the agent that reduces or eliminates a CNS effect of the therapeutic agent are provided as a single composition within a container in the kit. Suitable packaging and additional articles for use (e.g., measuring cup for liquid preparations, foil wrapping to minimize exposure to air, and the like) are known in the art and may be included in the kit.

VI. Methods

In another aspect, the invention provides methods, including methods of treatment, methods of decreasing the concentration of a substance in a physiological compartment (e.g., methods of delaying the onset or preventing chronic neurodegenerative diseases), methods of enhancing a therapeutic effect of a substance, methods of delaying, preventing, reducing or eliminating tolerance or dependence in an animal that is administered a substance, methods of drug wash-out, and methods for identifying modulators of blood-brain barrier transport proteins.

For simplicity, methods will be described in terms of reduction of a CNS effect of a substance. It is understood that the methods apply equally to exclusion of a substance from the fetal compartment, or reduction of fetal effects of a substance.

The term “animal” or “animal subject” as used herein includes humans as well as other mammals. The methods generally involve the administration of one or more drugs for the treatment of one or more diseases. Combinations of agents can be used to treat one disease or multiple diseases or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

A. Methods of Treating Conditions

In some embodiments, the invention provides a method of treating a condition by administering to an animal suffering from the condition an effective amount of a therapeutic agent and an amount of an BBB transport protein activator sufficient to reduce or eliminate a CNS effect of the therapeutic agent. In some embodiments, the activator reduces or eliminates a plurality of CNS effects of the therapeutic agent. In some embodiments the animal is a mammal, e.g., a human.

The therapeutic agent and the BBB transport protein activator are co-administered. “Co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present. Thus, in some embodiments, the BBB transport protein activator are administered in a single composition. In some embodiments, the therapeutic agent and the BBB transport protein activator are admixed in the composition. Typically, the therapeutic agent is present in the composition in an amount sufficient to produce a therapeutic effect, and the BBB transport protein activator is present in the composition in an amount sufficient to reduce a central nervous system effect of the therapeutic agent. In some embodiments, the therapeutic agent is present in an amount sufficient to exert a therapeutic effect and the BBB transport protein activator is present in an amount sufficient to decrease a CNS effect of the therapeutic agent by an average of at least about 5, 10, 15, 20, 25, 30, 40, 50, 60, 70, 80, 90, more than 90%, or substantially eliminate a CNS effect, compared to the effect without the BBB transport protein activator.

Administration of the therapeutic agent and the agent that reduces or eliminates at least one CNS effect of the therapeutic agent may be any suitable means. If the agents are administered as separate compositions, they may be administered by the same route or by different routes. If the agents are administered in a single composition, they may be administered by any suitable route. In some embodiments, the agents are administered as a single composition by oral administration. In some embodiments, the agents are administered as a single composition by transdermal administration. In some embodiments, the agents are administered as a single composition by injection.

In some embodiments, the agent that reduces or eliminates a side effect of a therapeutic agent is a BBB transport protein modulator BBB transport protein modulators are as described herein. In some embodiments, a polyphenol is used. In some embodiments, a flavonoid is used. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin, kaempferol, or galangin. In some embodiments, the flavonoid is quercetin. Dosages are as provided for compositions. Typically, the daily dosage of the BBB transport protein modulator will be about 0.5-100 mg/kg.

The therapeutic agent may be any therapeutic agent described herein. In some embodiments, the therapeutic agent is an antihypertensive, vasodilator, barbiturate, membrane stabilizer, cardiac stabilizer, glucocorticoid, or antiinfectives, as described herein.

The methods of the invention may be used for treatment of any suitable condition, e.g., diseases of the heart, circulation, lipoprotein metabolism, hemostasis and thrombosis, respiratory system, kidney, gastrointestinal tract, endocrine system, reproductive system, or hemopoeitic system, where one or more therapeutic agents are used that have CNS effects.

For example, in some embodiments, the methods of the invention include the treatment of hypertension in an animal by administering to an animal in need of treatment an effective amount of an antihypertensive and an effective amount of an agent that reduces or eliminates a CNS effect of the hypertensive. Another exemplary embodiment is the treatment or prevention of infection in an animal by administering to an animal in need of treatment or prevention of infection an effective amount of an antiinfective agent and an effective amount of an agent that reduces or eliminates a CNS effect of the antiinfective agent.

When a therapeutic agent and an agent that reduces or eliminates a CNS effect of the therapeutic agent are used in combination, any suitable ratio of the two agents, e.g., molar ratio, wt/wt ration, wt/volume ratio, or volume/volume ratio, as described herein, may be used.

B. Methods of Decreasing the Concentration of a Substance in a Physiological Compartment

The invention provides methods for reducing the concentration of a substance in a physiological compartment by selectively increasing efflux of the substance from the physiological compartment to an external environment. The physiological compartment preferably is a central nervous system or a fetal compartment.

In some embodiments, compositions of the invention may be administered chronically to an individual in order to prevent, delay the appearance, or slow or halt the progression of a chronic neurodegenerative condition. In some embodiments, compositions of the invention may be administered chronically to an individual in order to remove from the CNS one or more substances associated with a chronic neurodegenerative condition. In some embodiments, the neurodegenerative condition is prion disease, Alzheimer's disease (AD), Parkinson's disease (PD), Huntington's disease (HD), ALS, multiple sclerosis, transverse myelitis, motor neuron disease, Pick's disease, tuberous sclerosis, lysosomal storage disorders, Canavan's disease, Rett's syndrome, spinocerebellar ataxias, Friedreich's ataxia, optic atrophy, or retinal degeneration. In some embodiments, the neurodegenerative disease is AD. In some embodiments, the substance associated with a neurodegenerative disease is amyloid beta. In some embodiments, a flavonoid is administered to the individual, such as quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the individual is a human and is chronically administered an amount of quercetin effective in removing amyloid beta from the CNS. In some embodiments, the quercetin is administered in a pharmaceutical composition with a pharmaceutically acceptable excipient at a dose of 100 mg-10,000 mg per day. Other dosages of quercetin, as described herein, may also be used.

C. Methods of Treating Pain.

The invention provides methods of treating pain.

As used herein the term “pain” may refer to all types of pain, including, but not limited to, traumatic pain, neuropathic pain, inflammatory pain, acute pain, chronic pain, organ or tissue pain, and pain associated with diseases. The International Association for the Study of Pain (“IASP”) defines pain as “an unpleasant sensory and emotional experience associated with actual and potential tissue damage, or described in terms of such damage or both.” Pain is classified in several manners, conventionally by location, duration, cause, frequency, and intensity.

Traumatic pain includes, but is not limited to, pain resulting from injury, post-surgical pain and inflammatory pain. Neuropathic pain may include, but is not limited to, neuropathic and idiopathic pain syndromes, and pain associated with neuropathy such as diabetic neuropathy, causalgia, brachial plexus avulsion, occipital neuralgia, fibromyalgia, gout, and other forms of neuralgia. Organ or tissue pain may include, but is not limited to, headache, ocular pain, corneal pain, bone pain, heart pain, skin/burn pain, lung pain, visceral pain (kidney, gall bladder, etc.), joint pain, dental pain, muscle pain, pelvic pain, and urogenital pain (e.g. vulvodynia and prostadynia). Pain associated with diseases may include, but is not limited to, pain associated with cancer, AIDS, arthritis, herpes and migraine. Pain may be of varying severity, i.e. mild, moderate and severe pain in acute and/or chronic modes.

Pain may be due to injury, strain or inflammation of tendons or ligaments and may be referred to as “soft tissue pain.” Some of the soft tissue pain conditions which afflict humans may include, but is not limited to, tennis elbow, frozen shoulder, carpal tunnel syndrome, plantar fascitis, and Achilles tendonitis. Tennis elbow is due to inflammation of the tendons of the hand gripping muscles where these tendons are attached to the elbow. This may result in pain at the elbow. Frozen shoulder is a stiffening of the ligaments around the shoulder joint which may come on after prolonged unaccustomed use of the arm. Carpal tunnel syndrome involves a nerve which passes through the carpal tunnel on the front of the wrist into the human hand. When this tunnel becomes inflamed it can press on the nerve causing shooting pain into the thumb and first two fingers. Plantar fascitis involves ligaments in the sole of the foot which can get inflamed leading to pain on the bottom of the heel while walking. Achilles tendonitis involves the Achilles tendon located at the back of the human ankle and which may become inflamed and painful.

Pain may also include chronic pain, such as but not limited to, neuropathic pain, and post-operative pain, chronic lower back pain, cluster headaches, herpes neuralgia, phantom limb pain, central pain, dental pain, neuropathic pain, visceral pain, surgical pain, bone injury pain, pain during labor and delivery, pain resulting from burns, including sunburn, post partum pain, migraine, angina pain, and genitourinary tract-related pain including cystitis, nociceptive pain or nociception.

Pain associated with inflammatory diseases includes, but is not limited to: organ transplant rejection; reoxygenation injury resulting from organ transplantation including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory diseases of the gum, including gingivitis and periodontitis; tuberculosis; leprosy; inflammatory diseases of the kidney, including uremic complications, glomerulonephritis and nephrosis; inflammatory diseases of the skin, including sclerodermatitis, psoriasis and eczema; inflammatory diseases of the central nervous system, including chronic demyelinating diseases of the nervous system, multiple sclerosis, AIDS-related neurodegeneration and Alzheimer s disease, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, diabetic cataract, glaucoma, retinopathy, nephropathy (such as microaluminuria and progressive diabetic nephropathy), polyneuropathy, mononeuropathies, autonomic neuropathy, gangrene of the feet, atherosclerotic coronary arterial disease, peripheral arterial disease, nonketotic hyperglycemic-hyperosmolar coma, foot ulcers, joint problems, and a skin or mucous membrane complication (such as an infection, a shin spot, a candidal infection or necrobiosis lipoidica diabeticorum); immune-complex vasculitis, and systemic lupus erythematosus (SLE); inflammatory diseases of the heart, such as cardiomyopathy, ischemic heart disease hypercholesterolemia, and atherosclerosis; as well as various other diseases that can have significant inflammatory components, including preeclampsia, chronic liver failure, brain and spinal cord trauma, and cancer. Pain can be associated with a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer. Arthritis is associated with pain and can be divided into inflammatory and non-inflammatory arthritis. Osteoarthritis is a non-inflammatory type of arthritis. Inflammatory arthritis can be, by way of example only, rheumatoid arthritis, gout, psoriatic arthritis, reactive arthritis, viral or post-viral arthritis, and spondylarthritis which may affect the spine as well as joints.

Methods of treating acute or chronic pain Any suitable type of pain, whether acute or chronic, may be treated by the methods of the invention. Thus, in some embodiments, the invention provides a method of treating an animal for pain by administering to an animal in pain an effective amount of an analgesic agent and an amount of a BBB transport protein activator sufficient to reduce a central nervous system effect of the analgesic agent. In some embodiments the animal is a mammal, e.g., a human. In some embodiments, the BBB transport protein activator is administered in an amount sufficient to substantially eliminate a central nervous system effect of the analgesic compound. In some embodiments, the analgesic agent and the BBB transport protein activator are co-administered, e.g., in a single composition. When administered in a single composition, in some embodiments, the analgesic is present in the composition in an amount sufficient to produce an analgesic effect, and the BBB transport protein activator is present in the composition in an amount sufficient to reduce a central nervous system effect of the analgesic. In some embodiments, e.g., where the agents are in a single composition, the therapeutic agent is present in an amount sufficient to exert a therapeutic effect and the BBB transport protein activator is present in an amount sufficient to decrease a CNS effect of the therapeutic agent by an average of at least about 5, 10, 15, 20, 25, 30, 40, 50, 60, 70, 80, 90, or more than 90%, compared to the side effect without the BBB transport protein activator. In some embodiments, the analgesic agent is administered in an amount sufficient to produce an analgesic effect, and the amount is different than the amount sufficient to produce an analgesic effect in the absence of administration of the BBB transport protein activator, e.g., the amount of the analgesic agent administered is lower than the amount sufficient to produce an analgesic effect in the absence of administration of the BBB transport protein activator. In some embodiments, the amount necessary to produce an analgesic effect in the presence of the BBB transport protein activator is less than 90, 80, 70, 60, 50, 40, 30, 20, or 10% of the amount necessary in the absence of the BBB transport protein activator. The analgesic agent and the BBB transport protein modulator may be administered by any suitable route; if they are in separate compositions they may be administered by different routes or the same route. If they are in the same composition, they may be administered by any suitable route, e.g., oral administration, administration by injection, or transdermal administration.

Individuals suffering from chronic pain often are administered more than one therapeutic agent. For example, combinations of opiods with NSAIDs or acetaminophen are common. Other combinations are as prescribed by the health care provider. It will be appreciated that the invention also provides for the use of more than one analgesic agent together with one or more agents that reduce or eliminate one or more CNS effect of one or more of the analgesic agents.

In some embodiments, the animal suffers from acute pain. In some embodiments, the animal suffers from chronic pain. The pain may be due to any of the conditions described herein. In some embodiments, the pain is idiopathic pain. In some embodiments, the pain is lower back pain, neck pain, head pain, headache pain, migraine headache pain, neuropathic pain, angina pain, premenstrual pain, post-surgical pain, burn pain, fibromyalgia pain, pain due to injury, joint pain, e.g., pain associated with osteoarthritis or rheumatoid arthritis, dental pain, muscle pain, pelvic pain, urogenital pain, or pain associated with cancer, AIDS, arthritis, herpes or migraine. Pain may be of any severity, i.e. mild, moderate and severe pain in acute and/or chronic modes.

In some embodiments, the BBB transport protein activator is an activator of P-gP. In some embodiments, the BBB transport protein activator includes a polyphenol. In some embodiments, the polyphenol is a flavonoid. The flavonoid can be any suitable flavonoid, e.g., any flavonoid that produces a desirable reduction in a CNS effect of the analgesic. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin, kaempferol, or galangin. In some embodiments, the flavonoid is quercetin.

The analgesic agent may be any suitable analgesic agent. The analgesic can be an opioid analgesic, a non-opioid analgesic, or a combination of an opioid and non-opioid analgesic (e.g., hydrocodone-acetaminophen, etc.). In some embodiments, the analgesic agent is selected from oxycodone, gabapentin, pregabalin, hydrocodone, fentanyl, hydromorphine, levorphenol, morphine, methadone, tramadol and topiramate. In some embodiments, the analgesic agent is selected from oxycodone or gabapentin. In some embodiments, the analgesic is oxycodone. In some embodiments, the analgesic is gabapentin.

The method may also include administration to the animal in pain another therapeutic agent besides the analgesic agent. Non-limiting examples include antinausea agents, amphetamines, antianxiolytics, and hypnotics.

In an exemplary embodiment, a human suffering from pain is co-administered a first composition containing an effective amount of an analgesic agent and a second composition containing an amount of a BBB transport protein activator sufficient to reduce or eliminate a CNS effect of the analgesic agent. In some embodiments, the first and second composition is the same composition. In some embodiments, the first and/or second composition further contains a pharmaceutically acceptable excipient. In some embodiments, administration of the first and/or second composition is oral. In some embodiments, administration of the first and/or second composition is intravenous (e.g., for postoperative pain). In some embodiments, administration of the first and/or second compositions is transdermal (e.g., for chronic pain). In some embodiments, the amount of BBB transport protein activator is also sufficient to measurably increase the analgesic effect of the analgesic agent, compared to administration of the analgesic agent alone, e.g., by about 5, 10, 15, 20, 25, 30, 40, 50, 60, 70, 80, 90, 100, or more than about 100%.

In some embodiments, a human suffering from pain is co-administered a composition containing an effective amount of an analgesic agent that is alfentanil, buprenorphine, butorphanol, codeine, dezocine, fentanyl, hydromorphone, levomethadyl acetate, levorphanol, meperidine, methadone, morphine sulfate, nalbuphine, oxycodone, oxymorphone, pentazocine, propoxyphene, remifentanil, sufentanil, tramadol; or analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin and a second composition containing an amount of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin effective to reduce or eliminate a CNS effect of the analgesic agent. In some embodiments, the first and second composition is the same composition. In some embodiments, the first and/or second composition further contains a pharmaceutically acceptable excipient. In some embodiments, administration of the first and/or second composition is oral. In some embodiments, administration of the first and/or second composition is intravenous (e.g., for post-operative pain). In some embodiments, administration of the first and/or second compositions is transdermal (e.g., for chronic pain). In some embodiments, the amount of BBB transport protein activator is also sufficient to measurably increase the analgesic effect of the analgesic agent, compared to administration of the analgesic agent alone, e.g., by about 5, 10, 15, 20, 25, 30, 40, 50, 60, 70, 80, 90, 100, or more than about 100%.

In some embodiments, a human suffering from pain is co-administered a composition containing an effective amount of an analgesic agent that is oxycodone, gabapentin, pregabalin, hydrocodone, fentanyl, hydromorphine, levorphenol, morphine, methadone, tramadol or topiramate and a second composition containing an amount of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin effective to reduce or eliminate a CNS effect of the analgesic agent. In some embodiments, the first and second composition is the same composition. In some embodiments, the first and/or second composition further contains a pharmaceutically acceptable excipient. In some embodiments, administration of the first and/or second composition is oral. In some embodiments, administration of the first and/or second composition is intravenous (e.g., for post-operative pain). In some embodiments, administration of the first and/or second compositions is transdermal (e.g., for chronic pain). In some embodiments, the amount of BBB transport protein activator is also sufficient to measurably increase the analgesic effect of the analgesic agent, compared to administration of the analgesic agent alone, e.g., by about 5, 10, 15, 20, 25, 30, 40, 50, 60, 70, 80, 90, 100, or more than about 100%

In some embodiments, a human suffering from pain is co-administered a composition containing an effective amount of an analgesic agent that is oxycodone, gabapentin, hydrocodone, methadone, or tramadol and a second composition containing an amount of quercetin, galangin, or kaempferol, sufficient to reduce or eliminate a CNS effect of the analgesic agent. In some embodiments, the first and second composition is the same composition. In some embodiments, the first and/or second composition further contains a pharmaceutically acceptable excipient. In some embodiments, administration of the first and/or second composition is oral. In some embodiments, administration of the first and/or second composition is intravenous (e.g., for post-operative pain). In some embodiments, administration of the first and/or second compositions is transdermal (e.g., for chronic pain). In some embodiments, the amount of BBB transport protein activator is also sufficient to measurably increase the analgesic effect of the analgesic agent, compared to administration of the analgesic agent alone, e.g., by about 5, 10, 15, 20, 25, 30, 40, 50, 60, 70, 80, 90, 100, or more than about 100%.

Thus, for example, in some embodiments, the invention provides methods of treatment for a human suffering from pain by administering to a human suffering from pain a first composition containing an effective amount of oxycodone and second composition containing an amount of quercetin sufficient to reduce or eliminate a CNS effect of the oxycodone, where the first and second compositions are the same or different. In some embodiments, the amount of quercetin is also sufficient to measurably increase the analgesic effect of the oxycodone, e.g., by about 5, 10, 15, 20, 25, 30, 40, 50, 60, 70, 80, 90, 100, or more than about 100%, compared to administration of the oxycodone alone. In some embodiments, the invention provides methods of treatment for a human suffering from pain by administering to a human suffering from pain a first composition containing an effective amount of hydrocodone and second composition containing an amount of quercetin sufficient to reduce or eliminate a CNS effect of the hydrocodone, where the first and second compositions are the same or different. In some embodiments, the amount of quercetin is also sufficient to measurably increase the analgesic effect of the hydrocodone, e.g., by about 5, 10, 15, 20, 25, 30, 40, 50, 60, 70, 80, 90, 100, or more than about 100%, compared to administration of the hydrocodone alone. In some embodiments, the invention provides methods of treatment for a human suffering from pain by administering to a human suffering from pain a first composition containing an effective amount of methadone and second composition containing an amount of quercetin sufficient to reduce or eliminate a CNS effect of the methadone, where the first and second compositions are the same or different. In some embodiments, the amount of quercetin is also sufficient to measurably increase the analgesic effect of the methadone, e.g., by about 5, 10, 15, 20, 25, 30, 40, 50, 60, 70, 80, 90, 100, or more than about 100%, compared to administration of the methadone alone. In some embodiments, the invention provides methods of treatment for a human suffering from pain by administering to a human suffering from pain a first composition containing an effective amount of tramadol and second composition containing an amount of quercetin sufficient to reduce or eliminate a CNS effect of the tramadol, where the first and second compositions are the same or different. In some embodiments, the amount of quercetin is also sufficient to measurably increase the analgesic effect of the tramadol, e.g., by about 5, 10, 15, 20, 25, 30, 40, 50, 60, 70, 80, 90, 100, or more than about 100%, compared to administration of the tramadol alone. In some embodiments, the invention provides methods of treatment for a human suffering from pain by administering to a human suffering from pain a first composition containing an effective amount of gabapentin and second composition containing an amount of quercetin sufficient to reduce or eliminate a CNS effect of the gabapentin, where the first and second compositions are the same or different. In some embodiments, the amount of quercetin is also sufficient to measurably increase the analgesic effect of the gabapentin, e.g., by about 5, 10, 15, 20, 25, 30, 40, 50, 60, 70, 80, 90, 100, or more than about 100%, compared to administration of the gabapentin alone. In some embodiments, the invention provides methods of treatment for a human suffering from pain by administering to a human suffering from pain a first composition containing an effective amount of lorazepam and second composition containing an amount of quercetin sufficient to reduce or eliminate a CNS effect of the lorazepam, where the first and second compositions are the same or different. In some embodiments, the amount of quercetin is also sufficient to measurably increase the analgesic effect of the lorazepam, e.g., by about 5, 10, 15, 20, 25, 30, 40, 50, 60, 70, 80, 90, 100, or more than about 100%, compared to administration of the lorazepam alone. In some embodiments, the invention provides methods of treatment for a human suffering from pain by administering to a human suffering from pain a first composition containing an effective amount of cyclobenzaprine hydrochloride and second composition containing an amount of quercetin sufficient to reduce or eliminate a CNS effect of the cyclobenzaprine hydrochloride, where the first and second compositions are the same or different. In some embodiments, the amount of quercetin is also sufficient to measurably increase the analgesic effect of the cyclobenzaprine hydrochloride, e.g., by about 5, 10, 15, 20, 25, 30, 40, 50, 60, 70, 80, 90, 100, or more than about 100%, compared to administration of the cyclobenzaprine hydrochloride alone. In some embodiments, the invention provides methods of treatment for a human suffering from pain by administering to a human suffering from pain a first composition containing an effective amount of carisoprodol and second composition containing an amount of quercetin sufficient to reduce or eliminate a CNS effect of the carisoprodol, where the first and second compositions are the same or different. In some embodiments, the amount of quercetin is also sufficient to measurably increase the analgesic effect of the carisoprodol, e.g., by about 5, 10, 15, 20, 25, 30, 40, 50, 60, 70, 80, 90, 100, or more than about 100%, compared to administration of the carisoprodol alone. In some of these embodiments, administration for one or both compositions (if different) is oral. For some of these embodiments, administration for one or both compositions (if different) is transdermal. For some of these embodiments, administration for one or both compositions (if different) is by injection (e.g., intravenous).

In some exemplary embodiments, the invention provides methods of treatment for a human suffering from pain by orally administering to a human suffering from pain a composition containing an effective amount of oxycodone admixed with an amount of quercetin sufficient to reduce or eliminate a CNS effect of the oxycodone, optionally also containing a pharmaceutically acceptable excipient. In some embodiments, the amount of quercetin is also sufficient to measurably increase the analgesic effect of the oxycodone, e.g., by about 5, 10, 15, 20, 25, 30, 40, 50, 60, 70, 80, 90, 100, or more than about 100%, compared to administration of the oxycodone alone. In some exemplary embodiments, the invention provides methods of treatment for a human suffering from pain by orally administering to a human suffering from pain a composition containing an effective amount of hydrocodone admixed with an amount of quercetin sufficient to reduce or eliminate a CNS effect of the hydrocodone, optionally also containing a pharmaceutically acceptable excipient. In some embodiments, the amount of quercetin is also sufficient to measurably increase the analgesic effect of the hydrocodone, e.g., by about 5, 10, 15, 20, 25, 30, 40, 50, 60, 70, 80, 90, 100, or more than about 100%, compared to administration of the hydrocodone alone. In some exemplary embodiments, the invention provides methods of treatment for a human suffering from pain by orally administering to a human suffering from pain a composition containing an effective amount of tramadol admixed with an amount of quercetin sufficient to reduce or eliminate a CNS effect of the tramadol, optionally also containing a pharmaceutically acceptable excipient. In some embodiments, the amount of quercetin is also sufficient to measurably increase the analgesic effect of the tramadol, e.g., by about 5, 10, 15, 20, 25, 30, 40, 50, 60, 70, 80, 90, 100, or more than about 100%, compared to administration of the tramadol alone. In some exemplary embodiments, the invention provides methods of treatment for a human suffering from pain by orally administering to a human suffering from pain a composition containing an effective amount of methadone admixed with an amount of quercetin sufficient to reduce or eliminate a CNS effect of the methadone, optionally also containing a pharmaceutically acceptable excipient. In some embodiments, the amount of quercetin is also sufficient to measurably increase the analgesic effect of the methadone, e.g., by about 5, 10, 15, 20, 25, 30, 40, 50, 60, 70, 80, 90, 100, or more than about 100%, compared to administration of the methadone alone. In some exemplary embodiments, the invention provides methods of treatment for a human suffering from pain by orally administering to a human suffering from pain a composition containing an effective amount of gabapentin admixed with an amount of quercetin sufficient to reduce or eliminate a CNS effect of the gabapentin, optionally also containing a pharmaceutically acceptable excipient. In some embodiments, the amount of quercetin is also sufficient to measurably increase the analgesic effect of the gabapentin, e.g., by about 5, 10, 15, 20, 25, 30, 40, 50, 60, 70, 80, 90, 100, or more than about 100%, compared to administration of the gabapentin alone. In some exemplary embodiments, the invention provides methods of treatment for a human suffering from pain by orally administering to a human suffering from pain a composition containing an effective amount of lorazepam admixed with an amount of quercetin sufficient to reduce or eliminate a CNS effect of the lorazepam, optionally also containing a pharmaceutically acceptable excipient. In some embodiments, the amount of quercetin is also sufficient to measurably increase the analgesic effect of the lorazepam, e.g., by about 5, 10, 15, 20, 25, 30, 40, 50, 60, 70, 80, 90, 100, or more than about 100%, compared to administration of the lorazepam alone. In some exemplary embodiments, the invention provides methods of treatment for a human suffering from pain by orally administering to a human suffering from pain a composition containing an effective amount of cyclobenzaprine hydrochloride admixed with an amount of quercetin sufficient to reduce or eliminate a CNS effect of the cyclobenzaprine hydrochloride, optionally also containing a pharmaceutically acceptable excipient. In some embodiments, the amount of quercetin is also sufficient to measurably increase the analgesic effect of the cyclobenzaprine hydrochloride, e.g., by about 5, 10, 15, 20, 25, 30, 40, 50, 60, 70, 80, 90, 100, or more than about 100%, compared to administration of the cyclobenzaprine hydrochloride alone. In some exemplary embodiments, the invention provides methods of treatment for a human suffering from pain by orally administering to a human suffering from pain a composition containing an effective amount of carisoprodol admixed with an amount of quercetin sufficient to reduce or eliminate a CNS effect of the carisoprodol, optionally also containing a pharmaceutically acceptable excipient. In some embodiments, the amount of quercetin is also sufficient to measurably increase the analgesic effect of the carisoprodol, e.g., by about 5, 10, 15, 20, 25, 30, 40, 50, 60, 70, 80, 90, 100, or more than about 100%, compared to administration of the carisoprodol alone.

Methods of treating pain with reduction or elimination of tolerance and/or dependence One major problem facing sufferers of chronic pain is that many of the most effective analgesic agents, e.g., the opioids, also cause tolerance and/or dependence, necessitating increasing doses for the same analgesic effect as well as often causing withdrawal symptoms upon cessation or reduction of the dose of the analgesic agent. The methods of the invention are useful in reducing or eliminating tolerance and/or dependence to an analgesic agent. The methods may be used at the start of the use of the analgesic agent, or may be used after tolerance and/or dependence have occurred, in order to reduce or eliminate tolerance and/or dependence. Thus, in some embodiments, the methods of the invention allow a reduction in dose of the analgesic agent in a person who has chronically taken the agent, with no or minor reduction in analgesic effect, and/or with no or minor withdrawal symptoms. In other embodiments, the methods of the invention allow chronic administration of an analgesic agent to an individual with little or no development of tolerance or dependence, thus with little or no dose escalation.

Thus, in some embodiments, the invention provides a method of controlling chronic pain in an animal by co-administering to an animal suffering from chronic pain: (i) an effective amount of an analgesic agent; and (ii) an amount of a BBB transport protein modulator, e.g., activator, sufficient to prevent or delay the development of tolerance and/or dependence to the analgesic agent in the animal. In some embodiments, the analgesic agent is administered for a period of time before co-administration of the BBB transport protein modulator, e.g., activator, so that development of tolerance and/or dependence may have occurred. In some embodiments, the animal is a mammal. In some embodiments, the mammal is a human. In some embodiments, the amount of the BBB transport protein modulator is sufficient to reduce the amount of analgesic necessary for pain relief, compared to the amount necessary without the BBB transport protein modulator. In some embodiments, the analgesic agent is an opioid analgesic agent. In some embodiments the BBB transport modulator is a polyphenol, e.g., a flavonoid. In some embodiments, the analgesic agent and the BBB transport protein modulator are co-administered in a single composition, e.g., a composition in which they are admixed. In some embodiments, the analgesic agent is selected from the group consisting of alfentanil, buprenorphine, butorphanol, codeine, dezocine, fentanyl, hydromorphone, levomethadyl acetate, levorphanol, meperidine, methadone, morphine sulfate, nalbuphine, oxycodone, oxymorphone, pentazocine, propoxyphene, remifentanil, sufentanil, tramadol; or analgesic combinations such as codeine/acetaminophen, codeine/aspirin, hydrocodone/acetaminophen, hydrocodone/ibuprofen, oxycodone/acetaminophen, oxycodone/aspirin, propoxyphene/aspirin. In some embodiments, the analgesic agent is selected from the group consisting of oxycodone hydrocodone, fentanyl, hydromorphone, levorphenol, morphine, methadone, and tramadol. In some embodiments, the analgesic agent is selected from the group consisting of hydrocodone, tramadol, oxycodone, and methadone. In some embodiments, the analgesic agent is hydrocodone. In some embodiments, the analgesic agent is tramadol. In some embodiments, the analgesic agent is oxycodone. In some embodiments, the analgesic agent is methadone. In some embodiments the BBB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin.

D. Wash-Out Methods

The invention further provides methods of reversing one or more CNS effects of a substance by administering a BBB transport protein activator to an animal that has received an amount of the substance sufficient to produce one or more CNS effects. The methods are especially useful in a situation where it is desired to rapidly reverse one or more CNS effects of a substance, e.g., in an overdose situation or to enhance recovery from general anesthesia. Any suitable BBB transport protein described herein may be used.

In some embodiments, the invention provides a method for reversing a CNS effect of an agent in a human by administering to the human an amount of a BBB transport protein modulator sufficient to partially or completely reverse a central nervous system effect of the agent, where the human has received an amount of said agent sufficient to produce a central nervous system effect. In some embodiments, the agent is a general anesthetic. Examples of general anesthetics include, but not limited to, desflurane, dexmedetomidine, diazepam, droperidol, enflurane, etomidate, halothane, isoflurane, ketamine, lorazepam, methohexital, methoxyflurane, midazolam, nitrous Oxide propofol, sevoflurane, and thiopental. In some embodiments, the human has received an overdose of the agent producing the CNS effect. In some embodiments, the individual continues to experience peripheral effects of the agent. In some embodiments, the BBB transport protein modulator is a polyphenol, such as a flavonoid. In some embodiments, the flavonoid is quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, or epicatechin. In some embodiments, the flavonoid is quercetin. Typically, the flavonoid will be administered by injection, e.g., intravenously or intraperitoneally, in a dose sufficient to partially or completely reverse a CNS effect of the substance. Such a dose in a human can be, e.g., about 0.1-100 gm, or about 0.5-50 gm, or about 1-20 gm, or 1, 2, 3, 4, 5, 6, 7, 8, 9, 10, 12, 14, 16, 18, or 20 gm. In general, the dose can be 0.01-1.5 gm/kg.

E. Methods of Identifying a Transport Protein Modulator

A further aspect of the invention is a method of identifying a transport protein modulator. A drug is administered in an appropriate animal model in the presence and absence of a test compound and the concentration of the drug in a biological sample is measured. The test compound is identified as a transport protein modulator if the concentration of the drug in the biological sample is lower in the presence of the test compound. In some embodiments, the biological sample may be intraventricular samples, amniotic fluid, chorionic samples or brain parenchymal samples. Moreover, the animal model may be a rodent, such as mice or rats, or a primate, horse, dog, sheep, goat, rabbit, or chicken. In other embodiments, the animal model possesses a mutant form of a blood brain and/or placental transporter.

F. Administration

The methods involve the administration of an agent that reduces or eliminates a CNS effect of a substance. In some embodiments, a therapeutic agent that produces a CNS effect is administered in combination with an agent that reduces the effects of a CNS effect of the therapeutic agent. In some embodiments, other agents are also administered, e.g., other therapeutic agents. When two or more agents are co-administered, they may be co-administered in any suitable manner, e.g., as separate compositions, in the same composition, by the same or by different routes of administration.

In some embodiments, the agent that reduces or eliminates a CNS effect of a substance is administered in a single dose. This may be the case, e.g., in wash-out methods where the agent is introduced into an animal to quickly lower the CNS effect of a substance already present in the body. Typically, such administration will be by injection, e.g., intravenous injection, in order to introduce the agent quickly. However, other routes may be used as appropriate. A single dose of an agent that reduces or eliminates a CNS effect of a substance may also be used when it is administered with the substance (e.g., a therapeutic agent that produces a CNS effect) for treatment of an acute condition.

In some embodiments, the agent that reduces or eliminates a CNS effect of a substance and/or therapeutic agent is administered in multiple doses. Dosing may be about once, twice, three times, four times, five times, six times, or more than six times per day. Dosing may be about once a month, once every two weeks, once a week, or once every other day. In one embodiment the drug is an analgesic. In another embodiment the analgesic compound and the transport protein activator are administered together about once per day to about 6 times per day. In another embodiment the administration of the analgesic compound and the transport protein activator continues for less than about 7 days. In yet another embodiment the administration continues for more than about 6, 10, 14, 28 days, two months, six months, or one year. In some cases, continuous dosing is achieved and maintained as long as necessary, e.g., intravenous administration of analgesic in a post-operative situation or for a terminally ill patient, or transdermal dosing for chronic pain.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic pain.

An effective amount of a transport protein modulator and an effective amount of a drug may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

The BBB transport protein modulator and the therapeutic agent may be administered in dosages as described herein (see, e.g., Compositions). Dosing ranges for therapeutic agents are known in the art. Dosing for the BBB transport modulator may be found by routine experimentation. For a flavonoid, e.g., quercetin, typical daily dose ranges are, e.g. about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg. In some embodiments, the daily dose of quercetin is about 100, 200, 300, 400, 500, 600, 700, 800, 900, or 1000 mg. In some embodiments, the daily dose of quercetin is 100 mg. In some embodiments, the daily dose of quercetin is 500 mg. In some embodiments, the daily dose of quercetin is 1000 mg. Daily dose range may depend on the form of flavonoid, e.g., the carbohydrate moities attached to the flavonoid, and/or factors with which the flavonoid is administered, as described herein. The serum half-life for, e.g., quercetin, is about 19-25 hours, so single dose accuracy is not crucial.

When a BBB transport modulator, e.g., a flavonoid such as quercetin, is administered in a composition that comprises one or more therapeutic agents, and the therapeutic agent has a shorter half-life than BBB transport modulator (e.g., tramadol, hydrocodone, and the like have shorter half-lives than quercetin), unit dose forms of the therapeutic agent and the BBB transport modulator may be adjusted accordingly. Thus, for example, if quercetin is given in a composition also containing, e.g., tramadol, a typical unit dose form is, e.g., 50 mg tramadol/100 mg quercetin, or 50 mg tramadol/500 mg quercetin. See e.g., Compositions.

The Table, below, provides exemplary dosing schemes for selected analgesic agents and quercetin. These dosages are provided by way of example only and do not limit the invention.

| Therapeutic Agent (A) + | Per Dose (A:Q)* | | Per Day (A:Q) | |
|---|---|---|---|---|
| Quercetin.dihydrate (Q) | ˜mole:mole | ˜mg:mg | ˜mole:mole | ˜mg:mg |
| Vicodin | 0.006:1 | 10:1000 | 0.01:1 | 30:2000 |
| TID (hydrocodone | | | | |
| bitartrate - 5 mg per tablet) | | | | |
| Tramadol | 0.1:1 | 100:1000 | 0.2:1-0.3:1 | 400-600:2000 |
| OxyContin | 0.07:1 | 80:1000 | 0.1:1 | 240:2000 |
| Methadone | 0.04:1 | 40:1000 | 0.2:1 | 400:2000 |
| Gabapentin | 0.6:1 | 300:1000 | 0.8:1 | 900:2000 |
| | 1.75:1 | 900:1000 | 2.6:1 | 2700:2000 |
| Lorazepam (Ativan) | 0.001:1 | 1:1000 | 0.001:1 | 3:2000 |
| Cyclobnzaprine | 0.01:1 | 10:1000 | 0.01:1 | 30:2000 |
| hydrochloride | | | | |
| (Flexeril) | | | | |
| Carisoprodol (Soma) | 0.4:1 | 350:1000 | 0.6:1 | 1050:2000 |

*2000 mg quercetin daily, given in two divided doses, e.g., with two doses of the analgesic. Some doses of analgesic were given without quercetin.

### EXAMPLES

### Example 1

### Human Study of the Effects of Quercetin (Q) and Analgesics

An empiric trial on the effects of oral quercetin (Q) on sedation, concentration, and pain was conducted. Inclusion criteria included ongoing pain of at least 4/10 on the Likert scale, poor tolerance of current analgesic regimen (complaints of sedation, dizziness, inability to focus), and willingness to complete daily diaries.

Approximately 16 adult subjects with chronic pain were screened and 9 subjects were admitted to the trial. Their pain disorders included peripheral neuropathy (2), facial pain (2), cervical radiculopathy (2), lumbar spine disease (3). Their pre-existing medications included short acting opioids (Vicodin TID, Tramadol 50 mg Q4-6), high dose, long acting opioids (OxyContin 240 mg, Methadone 400 mg), Gabapentin (900 mg and 2700 mg), Ativan, Flexeril, and Soma 350 mg. Seven of the subjects were using at least two analgesic medications. Two subjects were using no current medications because of prior histories of sedation and dizziness during opioid trials.

Q (Sigma) 500 mg per gel capsule was compounded and supplied to all subjects by overnight mail. Subjects were instructed to complete daily diaries for 7 days and continue their baseline medications and regular activities. On approximately the 7th day, they were asked to begin twice daily dosing of 2 Q (1000 mg) capsules (total daily dose of Q, 2000 mg). Diaries were completed for 7 days. Individual diaries included rating sleep interference, focus, pain now, and worst pain over the prior 24 hours. Subjects were instructed that concomitant pain medications should not be altered without speaking with the investigator. Subjects were advised that they would be contacted by telephone every day or every other day to assess progress in the trial and any side effects associated with the addition of Q. At the end of the trial, patients were interviewed. They were asked to rate their satisfaction with the study medication (−2-+2) and its ability to modulate the CNS effects of their pain medications.

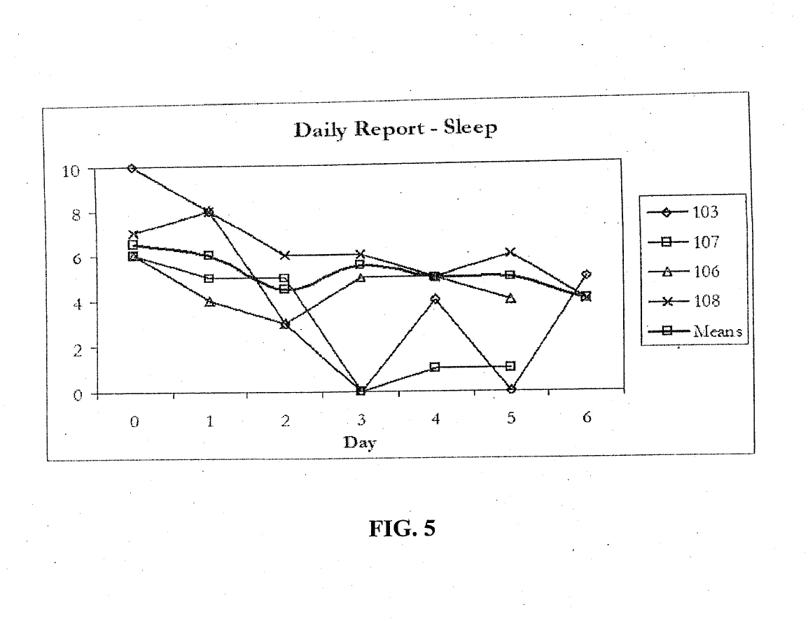

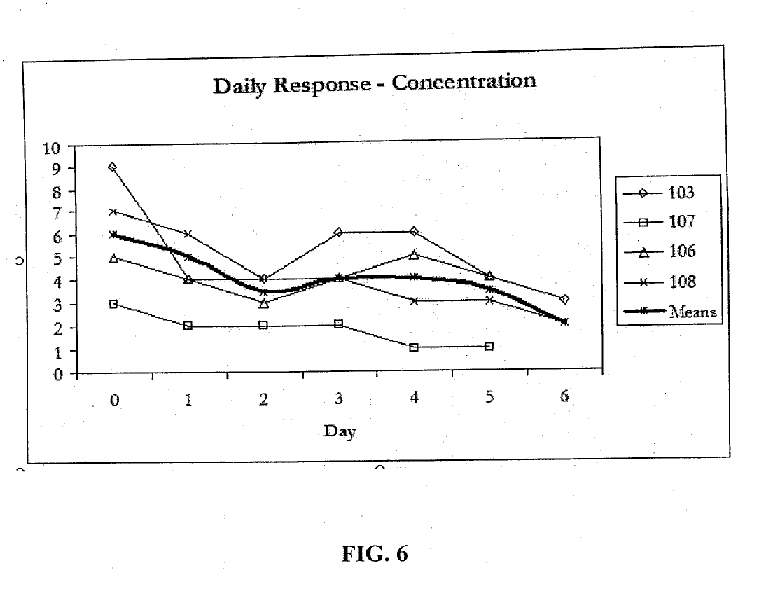

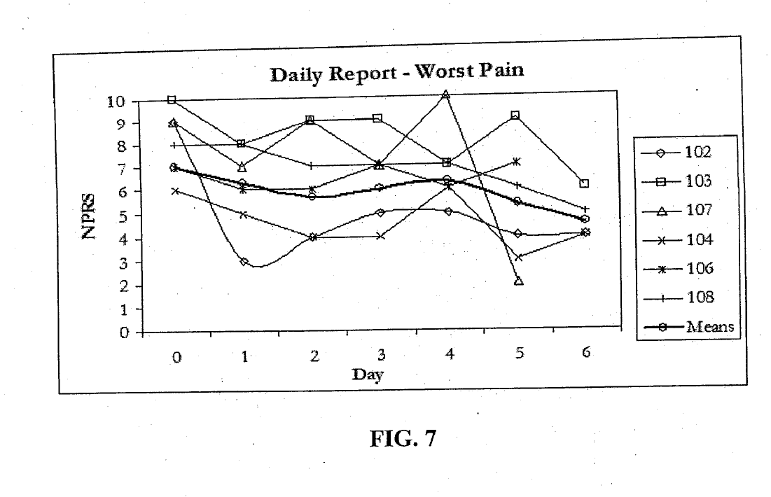

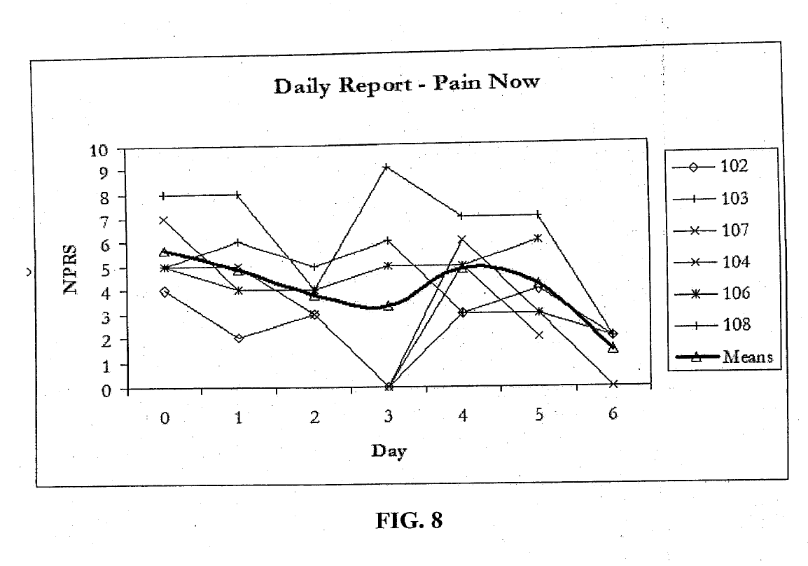

After taking Q, an overall improvement in sleep, pain and concentration was observed in all the patients. An overall improvement in sleep is depicted in FIG. 5 where y-axis depicts 1 as perfect sleep and 10 as worst. An overall improvement in the concentration (e.g. short term memory, focus, wakefulness etc.) was observed in all the patients, as shown in FIG. 6. In the graph, y-axis depicts 1 as perfect concentration and 10 as worst. An overall improvement in the worst pain in the last 24 hrs was observed in all the patients, as shown in FIG. 7. In the graph, y-axis depicts NPRS (numeric pain rating scale) as 1 for no pain and 10 as worst. An overall improvement in the pain was observed at the time the patients were called (“pain now”), as shown in FIG. 8.

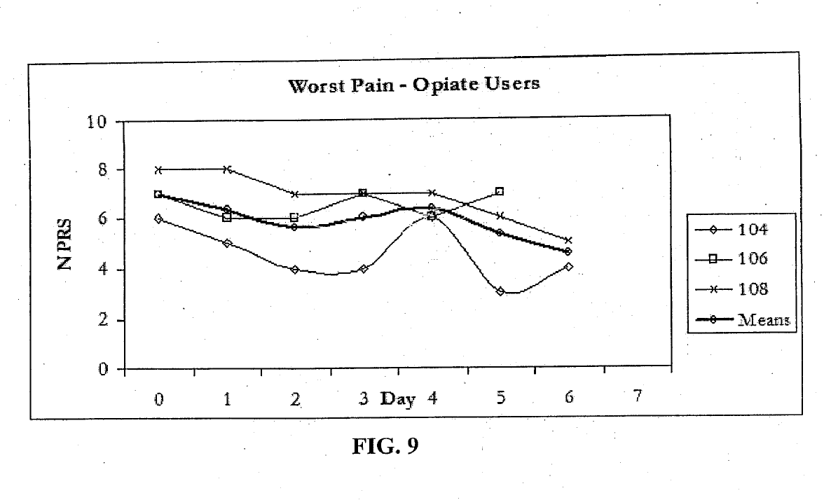

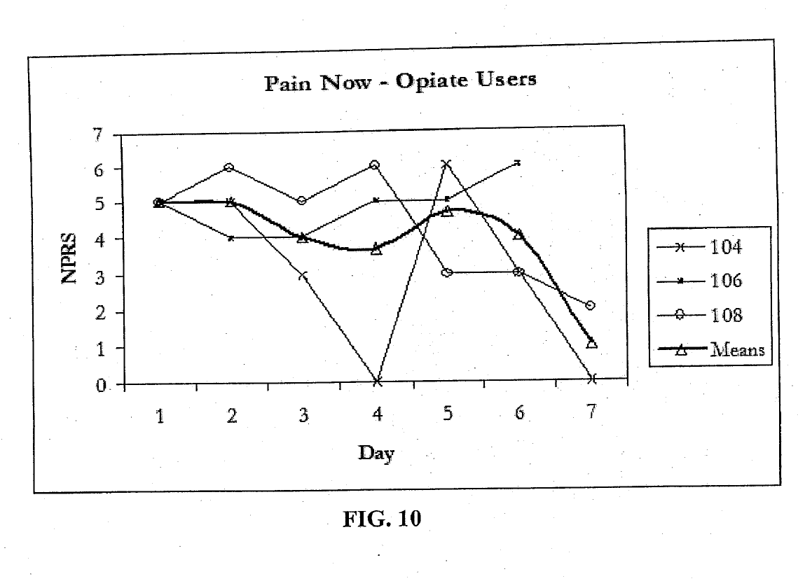

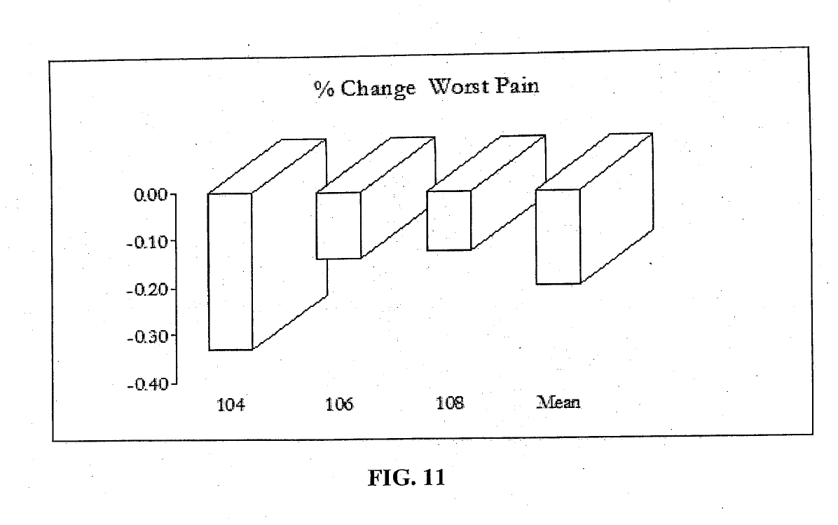

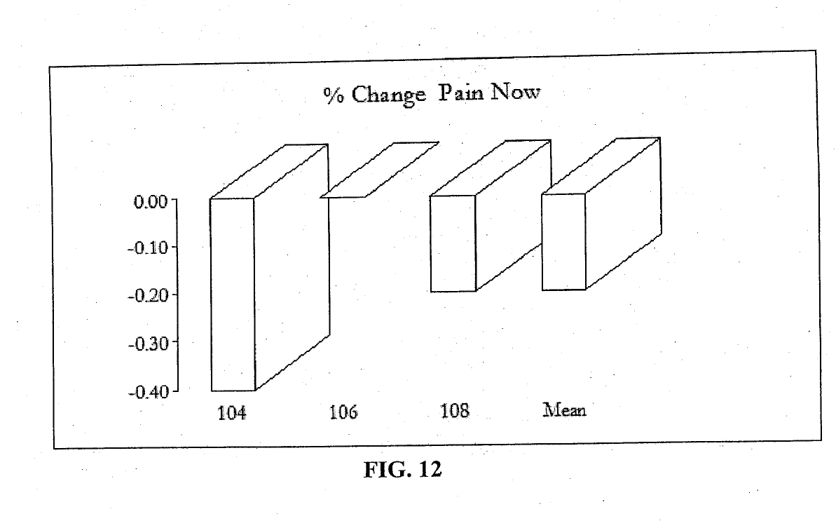

FIGS. 9-10 depict improvement in the conditions of three patients who were on opioids from the start. FIG. 9 depicts overall improvement in the worst pain in the last 24 hrs and FIG. 10 depicts overall improvement in the pain at the time patients were called. FIGS. 11 and 12 depict a % change in the worst pain in the last 24 hrs and % change in the pain at the time of the call, respectively, for the three patients.

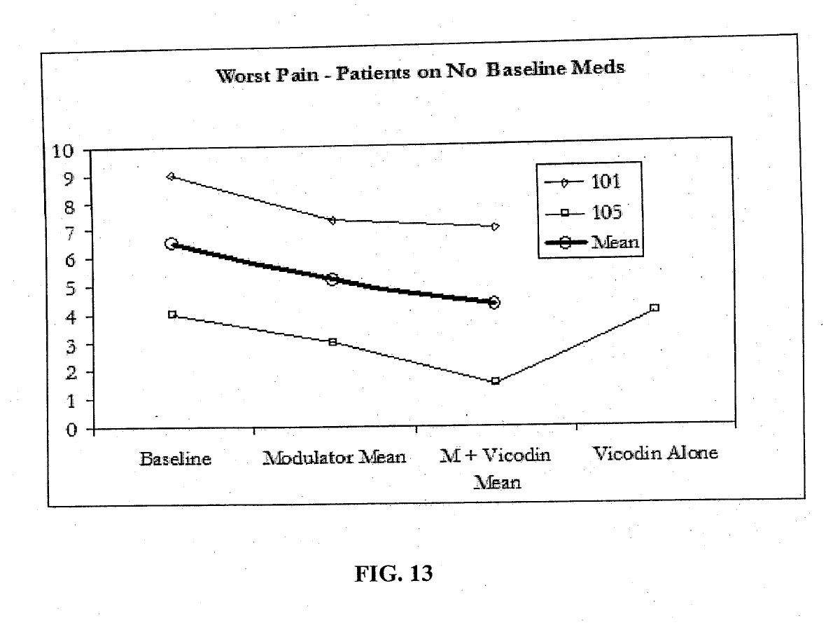

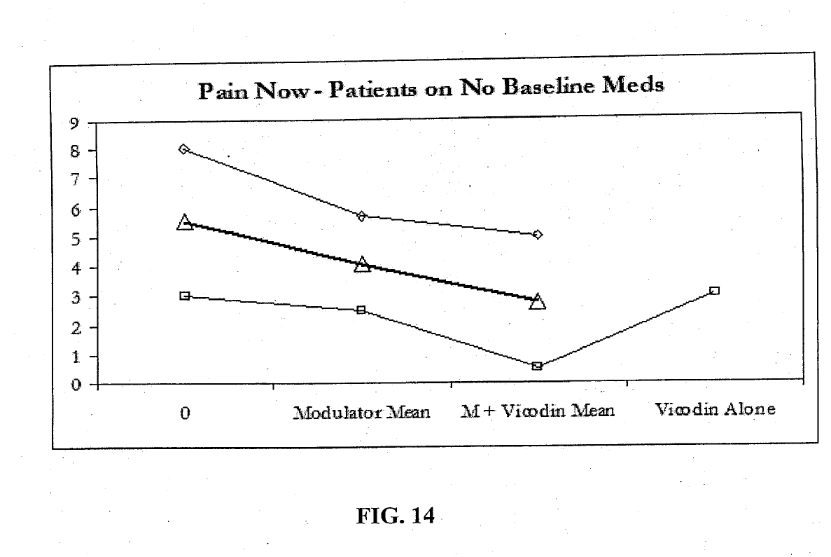

Two patients, both with the histories of poor tolerance of systemic medications were studied, as shown in FIGS. 13 and 14. Both the patients, who were not on baseline meds, were given Quercetin only which brought the pain down on the scale from 1-10. Administration of Vicodin along with Q brought the pain further down. One patient agreed to take Vicodin alone and reported increase in the pain as compared to Q alone or Q with Vicodin. FIG. 13 depicts the worst pain in the last 24 hrs and FIG. 14 depicts the pain at the time of the call.

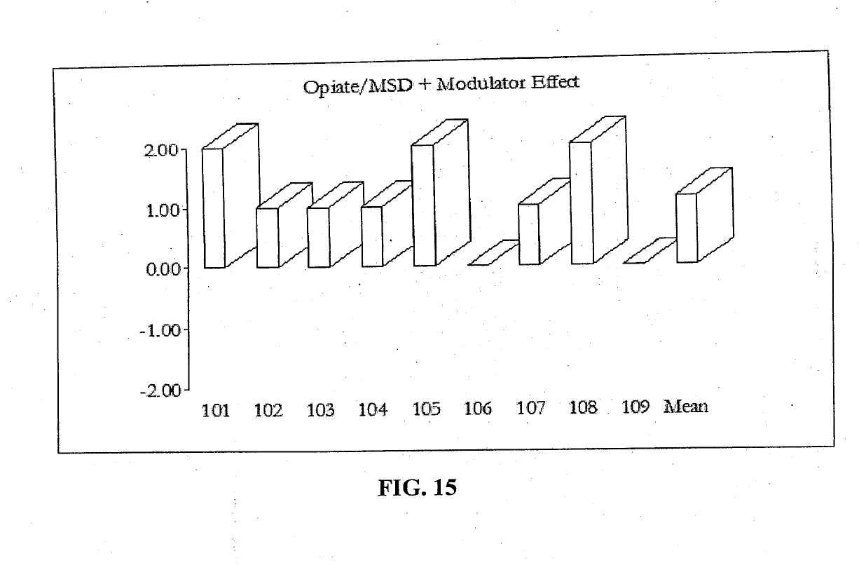

Global assessment of all the patients who were on opiate or MSD (membrane stabilizing drug) and modulator (Q) showed overall improvement in their condition, as shown in FIG. 15. On the scale of −2 to 2, none reported −2 and three reported 2. On an average there was an improvement in the pain in all the patients. CNS activation was noted in all the 4 opioid users and central withdrawal was noted in 3. An improvement in sleep, concentration and pain was observed in all the patients.

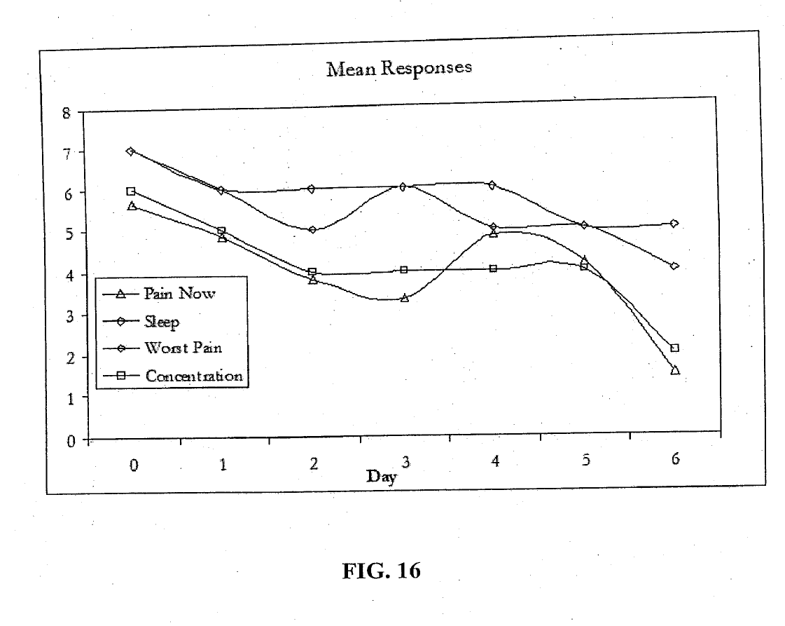

FIG. 16 shows mean improvement in all parameters measured over the course of the study, for all patients taking analgesic medications and Q. After 7 days of co-administration of Q and analgesics, mean ratings for pain now decreased by more than 70%, mean ratings for concentration improved by over 60%, and mean ratings for sleep and worst pain improved by more than 25%.

This Example illustrates that administration of a flavonoid (quercetin) in combination with one or more analgesics, in individuals experiencing chronic pain, resulted in improvement in all parameters measured (worst pain, pain now, concentration, sleep) of 25->70%.

### Example 2

### Reversal Effect of Modulator, Quercetin (Q), on Sedative Effects in Rodents

An anesthetic wake up test is used to assess the reversal effect of modulator, Q, on the sedative effects of barbiturates, opioids, and benzodiazepines. This is a single blind, randomized, controlled animal trial. Approximately 48 rodents are utilized throughout the study. Animals may be reused. However, a washout of 24 hours is required between exposures.

Twelve rodents are utilized in each portion of this trial. Intravenous barbiturate (e.g. diprivan, pentobarbital, or phenobarbital) anesthesia is induced and titrated to spontaneous but slow respirations and lack of response to painful stimulation. Supplemental oxygen is delivered. A maximum of 3 doses of intraperitoneal Q are tested (low, medium, high) along with placebo. Once administered rodents are monitored with the help of stopwatch for time to awakening and return to normal respiratory rate. Once awakened, rodents are tested for time to withdrawal from painful stimulus and performance on rotarod.

This study is repeated as a single agent trial with opioids (remifentanyl, fentanyl, morphine, etc) and benzodiazepines (diazepam, midazolam, lorazepam). This study is also repeated as a multi agent trial utilizing one opioid, one benzodiazepine, and one barbiturate.

### Example 3

### Identification of Efflux Transport Protein Modulators In Vitro

We are interested in the identification of molecules (including but not limited to excipients listed in the Pharmaceutical Additives Handbook, the Handbook of Pharmaceutical Excipients, or the Food and Drug Administration (FDA) Inactive Ingredient Guide) that would modulate transporter activity, for example by producing a significant increase in substrate efflux transport pumping. A screening process that integrates a P-gP enhancement assay with a software interface for data analysis will be used. P-gP substrate may include paclitaxel (an anti tumor agent) or other molecules which will produce cytotoxicity as an endpoint in this study. See Wang S W, Monagle J, McNulty C, Putnam D, Chen H. “Determination of P-glycoprotein inhibition by excipients and their combinations using an integrated high-throughput process.” J Pharm Sci. 2004 November; 93(11):2755-67.

Cell Culture and Cytotoxicity Assay

This assay is performed in (mouse fibroblast) NIH/3T3 and NIH-MDR-G185 cells (derived from 3T3 cells and transfected with the human MDR1 gene to overexpress human P-gP). Cells are nurtured in Dulbecco's modified Eagle's medium supplemented with necessary amino acids and energy substrate as necessary to ensure growth and they will be maintained in a humidified incubator at 37° C. with 5% carbon dioxide. Total growing time may be 72 hours or more.

Cell death due to modulation of P-gP activity and increased cytosolic paclitaxil or other cytotoxic agent is determined by an MTT assay [3,(4,5-dimethylthiazol-2-yl)2,5-diphenyltetrazolum bromide], a widely used method to assess cytotoxicity and cell viability in tissue culture. (IC₅₀) values for each excipient is determined by fitting the results to a sigmoidal curve. After IC₅₀ values (50% inhibitory concentration) are determined by fitting the data to a sigmoidal curve, these values are normalized relative to the no excipient value. These relative ratios rank the amount of P-gP enhancement (lack of cytotoxicity) due to each modulator.

Between 5-10 potential modulators with the greatest viability activity are chosen for combinatorial experiments based on the results of single-modulator studies. Dose-response studies are performed first for each of these modulators to determine the concentration range to use for the binary combination studies. Experiments will otherwise be performed according to the ‘single-modulator’ protocol using NIH/3T3 cells. For each modulator, up to four concentrations are tested, starting with the concentration used in the ‘single-modulator’ screen. IC₅₀ values are determined for each of the modulator concentrations and normalized to average saline values. Dose-response curves are generated as the normalized IC₅₀ versus concentration of modulator. Based on these dose-response curves, intermediate modulator concentrations corresponding to normalized IC₅₀ values are chosen for the binary combination studies.

P-gP Substrate Efflux Trials—In Vivo Pharmacokinetic Studies

Male wild-type FVB mdr1a/1b^{+/+} and P-gP-deficient knockout FVB mdr1a/1b^{-/-} mice (20-30 g) are obtained. Dose solutions of P-gP efflux substrate are prepared fresh using 0.9% saline as a vehicle. An appropriate amount of substrate is administered intravenously via the tail vein. The dosage amount is selected to provide sufficient analytical sensitivity while not resulting in sedation. The appropriate amount of substrate will vary depending on the compound, the weight, etc., of the subject to be treated.

At scheduled time points, mice are anesthetized with CO₂ and blood samples obtained by cardiac puncture. Blood is centrifuged to yield plasma. Brains are collected and the cerebellum/brain stem removed and discarded. The remaining brain tissue is frozen in liquid nitrogen. Individual brain-to-plasma and brain-to-free plasma concentration ratios and the group means and standard deviations are calculated using Microsoft Excel 2003 (Redmond, Wash.). Throughout the experiment, a blinded observer will note behavioral changes in the animals during the dosing portion of the study. Pharmacokinetic parameters are calculated using WinNonlin Enterprise software.

See Polli J W, Baughman T M, Humphreys J E, Jordan K H, Mote A L, Salisbury J A, Tippin T K, Serabjit-Singh C J. “P-glycoprotein influences the brain concentrations of cetirizine (Zyrtec), a second-generation non-sedating antihistamine.” J Pharm Sci. 2003 October; 92(10):2082-9, all of which are incorporated herein by reference.

### Example 4

### Quercetin (Flavonol-PgP Modulator) Ameliorates CNS Effects of Oxycodone in an Acute Pain Model

Opioids frequently produce adverse CNS side effects in ambulatory settings, providing a rationale for improving opioid analgesia by minimizing adverse effects. Quercetin is a flavonol which is shown herein to activate PgP efflux of pump ligands at the blood brain barrier. In this pilot study, the hypothesis that quercetin improves the tolerability and safety of oxycodone without impairing analgesia is tested. Healthy subjects (N=65) undergoing third molar removal are randomly allocated to receive 500 mg oral quercetin or matching placebo at 1 hr prior to surgery. All subjects receive 10 mg oral oxycodone immediately prior to surgery. As expected, pupil size decreases with increasing oxycodone plasma concentration. Oxycodone concentrations do not differ between groups prior to surgery, at 1 hr, or at 4 hr. Total Nausea and Vomiting Score (TNVS) is calculated for the time from dosing through 24 hr. More subjects in the quercetin group (67%) than in the placebo group (56%) experience “no” nausea and vomiting. Median TNVS is 0.39 (LNS5662) and 0.57 (placebo). No subjects in the quercetin group experience the most severe level of nausea or vomiting vs. 3 in the placebo group. Fewer subjects in the quercetin group (33.3%) than the placebo group (43.8%) request anti-emetic, and the placebo group tend to request anti-emetic sooner (280 vs 306 min). Importantly, the quercetin treated group have consistently lower mean pain intensity than the placebo group. All subjects except one request additional analgesic doses; there was no difference in median time to request for a second analgesic (120 and 131 min, respectively). Quercetin given 1 hr prior to oxycodone may ameliorate the severity of nausea and vomiting while not interfering with analgesic efficacy. The results show that administration of a flavinoid, such as quercetin, may improve opioid analgesia by minimizing adverse effects.

All of the methods disclosed and claimed herein can be made and executed without undue experimentation in light of the present disclosure. It will be apparent to those of skill in the art that variations may be applied without departing from the concept, spirit and scope of the invention. More specifically, it will be apparent that certain agents that both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A pharmaceutical composition comprising an analgesic agent, a blood brain barrier (BBB) transport protein activator and a pharmaceutically acceptable excipient, wherein the analgesic agent is present in an amount sufficient to produce an analgesic effect, and wherein the BBB transport protein activator is present in an amount sufficient to reduce a central nervous system (CNS) effect of the analgesic agent.

2. The composition of claim 1 wherein the BBB transport protein is an ABC transport protein.

3. The composition of claim 1 wherein the effect is selected from the group consisting of drowsiness, impaired concentration, sexual dysfunction, sleep disturbances, habituation, dependence, alteration of mood, respiratory depression, nausea, vomiting, dizziness memory impairment, neuronal dysfunction, neuronal death, visual disturbance, impaired mentation, tolerance, addiction, hallucinations, lethargy, myoclonic jerking, endocrinopathies, and combinations thereof.

4. The composition of claim 1 wherein a therapeutic effect of the therapeutic agent is increased at least about 10% compared to the therapeutic effect without the BBB transport protein activator, when the composition is administered to an animal.

5. The composition of claim 2 wherein the ABC transport protein is a P-gP.

6. The composition of claim 1 wherein the analgesic is selected from the group consisting of oxycodone, gabapentin, pregabalin, hydrocodone, fentanyl, hydromorphone, levorphenol, morphine, methadone, tramadol, topiramate, diacetyl morphine, codeine, olanzapine, hydrocortisone, prednisone, sufentanyl, alfentanyl, carbamazapine, lamotrigine, doxepin, and haloperidol.

7. The composition of claim 1 wherein the analgesic is selected from the group consisting of oxycodone and gabapentin.

8. The composition of claim 1 wherein the analgesic is oxycodone.

9. The composition of claim 1 wherein the analgesic is gabapentin.

10. The composition of claim 1 wherein the BBB transport protein activator is a polyphenol.

11. The composition of claim 10 wherein the BBB transport protein activator is a flavonoid.

12. The composition of claim 11 wherein the BBB transport protein activator is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

13. The composition of claim 12 wherein the BBB transport protein activator is quercetin.

14. The composition of claim 12 wherein the analgesic is selected from the group consisting of oxycodone, gabapentin, pregabalin, hydrocodone, fentanyl, hydromorphine, levorphenol, morphine, methadone, tramadol and topiramate.

15. The composition of claim 14 wherein the analgesic is selected from the group consisting of oxycodone and gabapentin.

16. The composition of claim 15 wherein the analgesic is oxycodone.

17. The composition of claim 15 wherein the analgesic is gabapentin.

18. The composition of claim 13 wherein the analgesic is selected from the group consisting of oxycodone and gabapentin.

19. The composition of claim 13 wherein the analgesic is oxycodone.

20. The composition of claim 19 wherein the oxycodone and the quercetin are present in a molar ratio of about 0.002:1 to 0.1:1.

21. The composition of claim 19 wherein the oxycodone is present at about 5-160 mg and the quercetin is present at about 10-500 mg.

22. The composition of claim 21 wherein the oxycodone is present at about 80 mg and the quercetin is present at about 500 mg.

23. The composition of claim 13 wherein the analgesic is gabapentin.

24. The composition of claim 23 wherein the gabapentin and the quercetin are present in a molar ratio of about 0.2:1 to 6:1

25. The composition of claim 23 wherein the gabapentin is present at about 100 to 800 mg and the quercetin is present at about 50-5000 mg.

26. The composition of claim 25 wherein the gabapentin is present at about 300 mg and the quercetin is present at about 150 mg.

27. The composition of claim 1 wherein the analgesic and the BBB transport protein activator are present in a molar ratio of about 0.001:1 to about 10:1.

28. The composition of claim 14 wherein the analgesic and the BBB transport protein activator are present in a molar ratio of about 0.001:1 to about 10:1.

29. The composition of claim 1 wherein the analgesic is present at about 0.001 to 500 mg and the BBB transport protein activator is present at about 10 to 1000 mg.

30. The composition of claim 1 wherein the central nervous system effect includes an effect selected from the group consisting of drowsiness, impaired concentration, sexual dysfunction, sleep disturbances, habituation, dependence, alteration of mood, respiratory depression, nausea, vomiting, dizziness memory impairment, neuronal dysfunction, neuronal death, visual disturbance, impaired mentation, tolerance, addiction, hallucinations, lethargy, myoclonic jerking, endocrinopathies, and combinations thereof.

31. The composition of claim 1 wherein the analgesic and the BBB transport protein activator are admixed.

32. A method of treating an animal for pain comprising administering to an animal in pain an effective amount of an analgesic agent and an amount of a BBB transport protein activator sufficient to reduce a central nervous system effect of the analgesic agent.

33. The method of claim 32 wherein the BBB transport protein activator is administered in an amount sufficient to substantially eliminate a central nervous system effect of the analgesic compound.

34. The method of claim 32 wherein the analgesic agent and the BBB transport protein activator are co-administered.

35. The method of claim 34 wherein the analgesic compound and the BBB transport protein activator are administered admixed in a single composition.

36. The method of claim 35 wherein the analgesic is present in the composition in an amount sufficient to produce an analgesic effect, and wherein the BBB transport protein activator is present in the composition in an amount sufficient to reduce a central nervous system effect of the analgesic.

37. The method of claim 35 wherein the therapeutic agent is present in an amount sufficient to exert a therapeutic effect and the BBB transport protein modulator is present in an amount sufficient to decrease a CNS effect of the therapeutic agent by an average of at least about 10%, compared to the side effect without the BBB transport protein modulator.

38. The method of claim 32 wherein the amount of analgesic agent is administered in an amount sufficient to produce an analgesic effect, and wherein said amount is different than the amount sufficient to produce an analgesic effect in the absence of administration of the BBB transport protein activator.

39. The method of 38 wherein the amount of analgesic agent administered is lower than the amount sufficient to produce an analgesic effect in the absence of administration of the BBB transport protein activator.

40. The method of claim 32 wherein the administration is oral administration.

41. The method of claim 32 wherein the administration is transdermal administration.

42. The method of claim 32 wherein the animal in pain suffers from chronic pain.

43. The method off claim 32 wherein the animal is a mammal.

44. The method of claim 32 wherein the animal is a human.

45. The method of claim 32 wherein the BBB transport protein activator is an activator of P-gP.

46. The method of claim 32 wherein the BBB transport protein activator comprises a polyphenol.

47. The method of claim 46 wherein the polyphenol is a flavonoid.

48. The method of claim 47 wherein the flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

49. The method of claim 48 wherein the flavonoid is quercetin.

50. The method of claim 32 wherein the analgesic is selected from the group consisting of oxycodone, gabapentin, pregabalin, hydrocodone, fentanyl, hydromorphine, levorphenol, morphine, methadone, tramadol and topiramate.

51. The method of claim 50 wherein the analgesic is selected from the group consisting of oxycodone and gabapentin.

52. The method of claim 51 wherein the analgesic is oxycodone.

53. The method of claim 51 wherein the analgesic is gabapentin.

54. The method of claim 49 wherein the analgesic is selected from the group consisting of oxycodone, gabapentin, pregabalin, hydrocodone, fentanyl, hydromorphine, levorphenol, morphine, methadone, tramadol and topiramate.

55. The method of claim 54 wherein the analgesic is selected from the group consisting of oxycodone and gabapentin.

56. The method of claim 54 wherein the analgesic is oxycodone.

57. The method of claim 54 wherein the analgesic is gabapentin.

58. The method of claim 34 wherein the analgesic compound and the BBB transport protein activator are administered together about once per day to about 6 times per day.

59. The method of claim 58 wherein the administration continues for less than about 7 days.

60. The method of claim 58 wherein the administration continues for more than about 6 days.

61. The method of claim 32 further comprising administering to the animal in pain another therapeutic agent.

62. The method of claim 61 wherein the other therapeutic agent is selected from the group consisting of antinausea agents, amphetamines, antianxiolytics, and hypnotics.

63. The method of claim 32 wherein the molar ratio of the amount of analgesic agent administered and the amount of BBB transport protein modulator administered is about 0.001:1 to about 10:1.

64. A method of controlling chronic pain comprising co-administering to an animal suffering from chronic pain
(i) an effective amount of an analgesic agent; and
(ii) an amount of a BBB transport protein modulator sufficient to prevent or delay the development of tolerance to the analgesic agent in the animal.

65. The method of claim 64 wherein the animal is a mammal

66. The method of claim 65 wherein the mammal is a human.

67. The method of claim 66 wherein the amount of the BBB transport protein modulator is sufficient to reduce the amount of analgesic necessary for pain relief.

68. The method of claim 66 wherein the analgesic agent is selected from the group consisting of oxycodone, gabapentin, pregabalin, hydrocodone, fentanyl, hydromorphine, levorphenol, morphine, methadone, tramadol and topiramate.

69. The method of claim 68 wherein the analgesic agent is oxycodone.

70. The method of claim 68 wherein the analgesic agent is gabapentin

71. The method of claim 64 wherein the BBB transport protein modulator is a polyphenol.

72. The method of claim 71 wherein the polyphenol is a flavonoid

73. The method of claim 72 wherein the flavonoid is selected from the group consisting of quercetin, isoquercetin, flavon, chrysin, apigenin, rhoifolin, diosmin, galangin, fisetin, morin, rutin, kaempferol, myricetin, taxifolin, naringenin, naringin, hesperetin, hesperidin, chalcone, phloretin, phlorizdin, genistein, biochanin A, catechin, and epicatechin.

74. The method of claim 73 wherein the flavonoid is quercetin.

75. The method of claim 64 wherein the analgesic agent and the BBB transport protein modulator are co-administered as admixed components of a single composition.
